(19) United States
(12) Patent Application Publication
(10) Pub. No.: US 2004/0235717 A1
(43) Pub. Date: Nov. 25, 2004

(54) Suppression of androgen receptor transactivation through new pathways to ar and ar coactivators and repressors

(76) Inventor: Chawnshang Chang, Pittsford, NY (US)

Correspondence Address: NEEDLE & ROSENBERG, P.C., SUITE 1000, 999 PEACHTREE STREET, ATLANTA, GA, 30309-3915, US

(21) Appl. No.: 10/473,939

(22) PCT Filed:
(86) PCT No.: PCT/US02/11086

Related U.S. Application Data
(60) Provisional application No. 60/282,266, filed on Apr. 6, 2001.
(60) Provisional application No. 60/365,060, filed on Mar. 13, 2002.

(51) Int. Cl. A61K 38/17; G01N 33/567; G01N 33/53

(52) U.S. Cl. 514/12; 435/7.2

(57) ABSTRACT

Disclosed are compositions and methods for related to signal transduction pathways related to androgen receptor.

1. This application claims priority to U.S. patent application Ser. No. 60/282,266 filed on Apr. 6, 2001 for “Suprression of Androgen Receptor Transactivation Through New Pathways to AR” which is incorporated by reference herein in its entirety, and to U.S. patent application Ser. No. 60/365,060 filed on Mar. 13, 2002 for “Signaling Pathways Related to Androgen Receptor” which is incorporated by reference herein in its entirety.

### I. BACKGROUND OF THE INVENTION

2. The androgen receptor (AR), a transcription factor, belongs to the nuclear receptor superfamily. Once bound to androgen, AR translocates into the nucleus, leading to activation of its target genes (Chang, C. S., Kokontis, J. & Liao, S. T. (1988) Science 240, 324-6). AR consists of the amino-terminal region that is involved in transcriptional activation, as well as the DNA-binding domain (DBD) and the ligand-binding domain (LBD) that are involved in androgen binding and receptor dimerization (Derynck et al. (1998) Cell 95, 737-740). It is generally accepted that AR plays an important role in the development of reproductive organs and in prostate cancer progression (Derynck et al. (1998) Cell 95, 737-740; Massagué. (1998) Ann Rev of Biochem 67,753-791). After binding to ligand, the activated AR is able to recognize palindromic DNA sequences, called androgen response elements (AREs), and form a complex with AR associated proteins to induce the expression of AR target genes. Several AR coregulators, androgen receptor associated proteins (ARAs) such as ARA24, ARA54, ARA55, ARA70, ARA160, Rb, TIFIIH and steroid receptor coactivator-1 (SRC-1), have been isolated and characterized (Yeh et al. (1999) Keio J Med 48, 87-92; Kang et al. (1999) J Biol Chem 274, 8570-6; Fujimoto et al. (1999) J Biol Chem 274, 8316-21; Yeh et al. (1996) Proc Natl Acad Sci USA 93, 5517-21; Miyamoto et al. (1998) Proc Natl Acad Sci USA 95, 11083-8; Yeh et al. (1998) Biochem Biophys Res Commun 248, 361-7; Miyamoto et al. (1998) Proc Natl Acad Sci USA 95, 7379-84; Yeh et al. (1998) Proc Natl Acad Sci USA 95, 5527-32). Results from these studies suggest that coregulators not only can enhance AR transactivation, but may also be able to increase the agonist activity of antiandrogens and 17-β estradiol (E2) in prostate cancer DU145 cells.

3. Reports have suggested that AR transactivation could be also induced by growth factors such as epidermal growth factor (EGF), insulin-like growth factor-1 (IGF-1), keratinocyte growth factor (KGF) (Culig et al. (1994) Cancer Res 54, 5474-8) and cytokines like interleukin-6 (IL-6) via an ligand-independent manner (Hobisch et al. (1998) Cancer Res 58, 4640-5). However, the range androgen dependent signal transduction pathways involving AR is not known. Disclosed herein are various signal transduction pathways that interact with AR, including PI3K pathways, Akt pathways, p21 pathways, TGF-β pathways, Smad3 pathways, Smad4 pathways, PTEN pathways, and specific IL6 pathways, and Herk/Neu pathways. Also disclosed are compositions and methods involving these various pathways for identifying modulators of androgen receptor activity and and modulators of androgen related cell growth. Also disclosed are modulators of these pathways, and methods of treating androgen related diseases, such as prostate cancer.

### II. SUMMARY OF THE INVENTION

4. In accordance with the purposes of this invention, as embodied and broadly described herein, this invention, in one aspect, relates to methods and compositions related to signal transduction pathways related to androgen receptor.

5. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not reflective of the invention, as claimed.

### III. BRIEF DESCRIPTION OF THE DRAWINGS

6. The accompanying drawings, which are incorporated in and constitute apart of this specification, illustrate several embodiments of the invention and together with the description, serve to explain the principles of the invention.

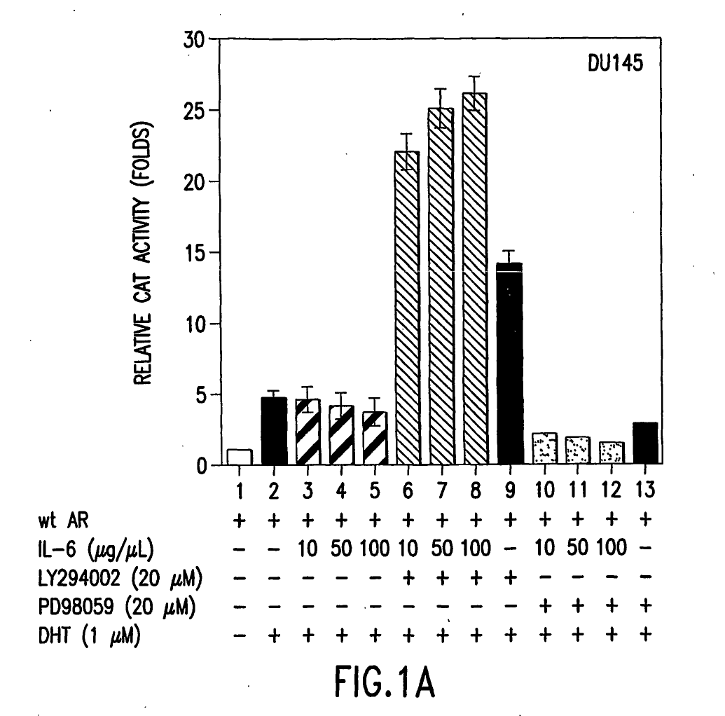

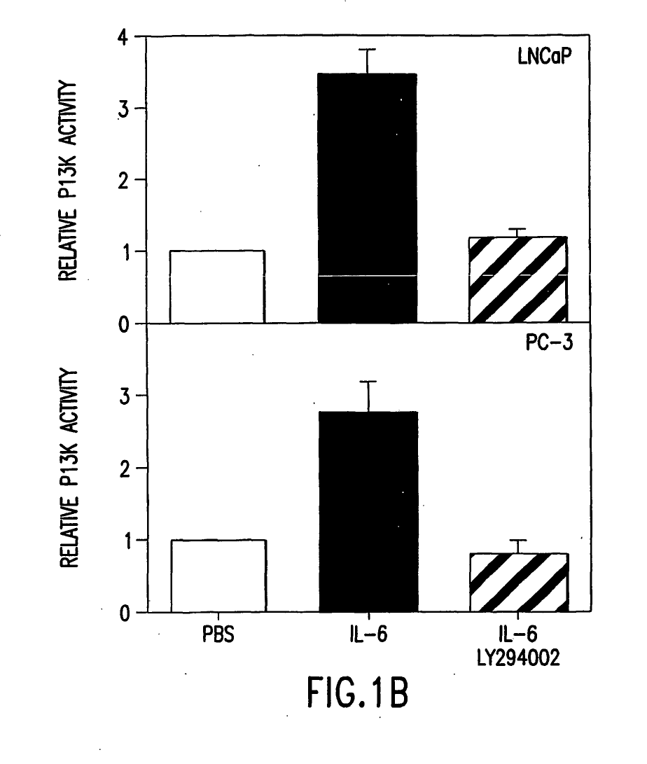

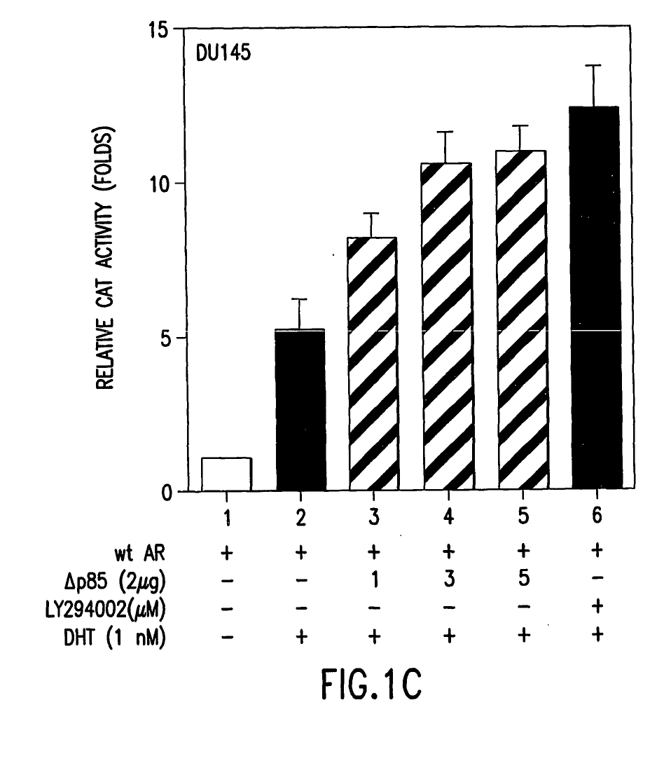

7. FIG. 1 shows induction and repression of AR transactivation by IL-6 via PI3K or MAPK. (a) Effect of IL-6 on AR transactivation. DU145 cells were transfected for 24 h with AR and MMTV-CAT reporter gene. After transfection, the cells were serum starved for 24 h , then 20 μM LY294002 or PD98059 was added to serum-free medium 30 min prior to IL-6 treatment. After 30 min of treatment with IL-6, DHT was added for another 24 h. The cells were then harvested and AR transactivation was measured by CAT activity. (b) Activation of PI3K activity by IL-6 in LNCaP, PC-3 and DU145 cells. After serum starvation for 24 h, LNCaP, PC-3 or DU145 cells were treated with 50 μg/ml IL-6 for 30 min, and then harvested. PI3K activity was measured by using phosphatidylinositol (PI) as a substrate. (c) Enhancement of AR transactivation through the inhibition of PI3K activity by Δp85 and LY294002. After 24 h transfection, the DU145 cells were treated with vehicle or LY294002 for 30 min prior to DHT treatment. The transactivation was measured by CAT activity. (d) Inhibition of AR transactivation by p110* in a dose dependent manner.

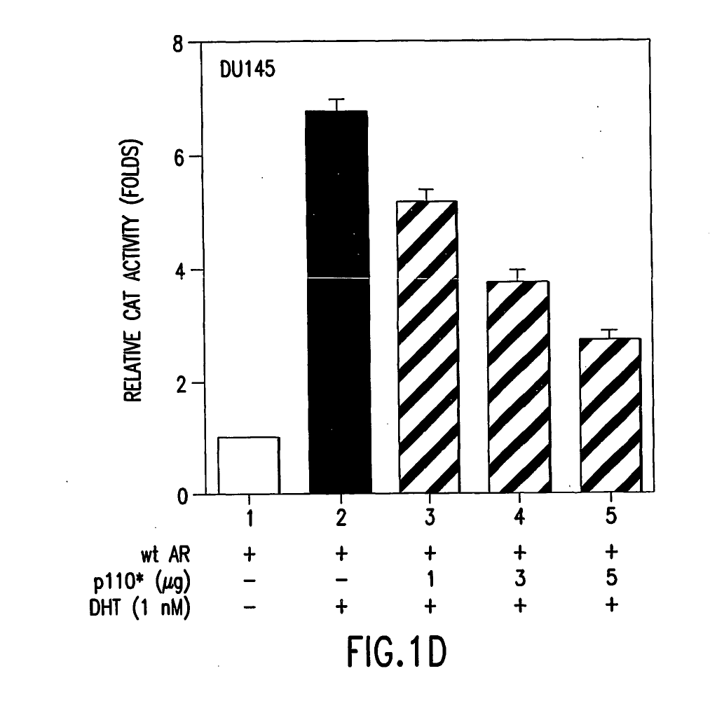

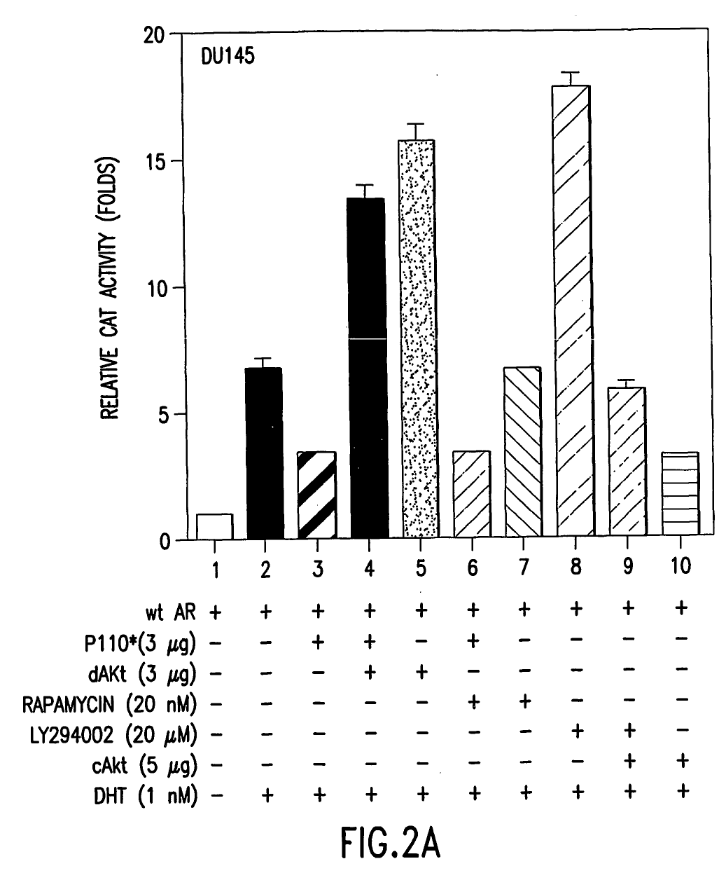

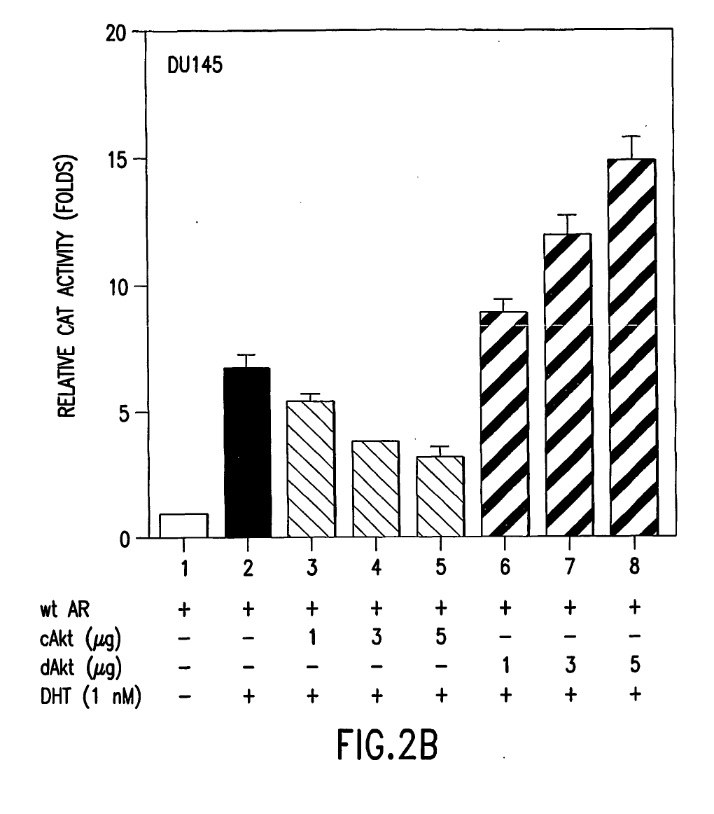

8. FIG. 2 show suppression of AR transactivation by Akt. (a) Suppression of AR transactivation by PI3K via Akt but not p70S6K. The DU145 cells were treated with LY294002 or rapamycin for 30 min prior to DHT treatment, the transactivation activity was determined after 24 h transfection. (b) Dose dependent inhibition of AR transactivation by cAkt or enhancement of AR transactivation by dAkt.

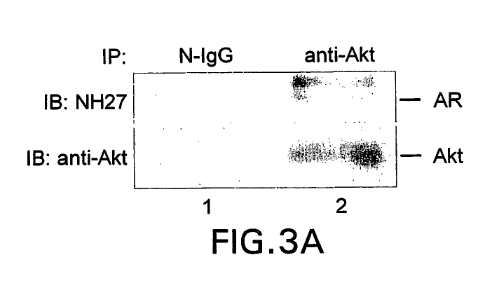

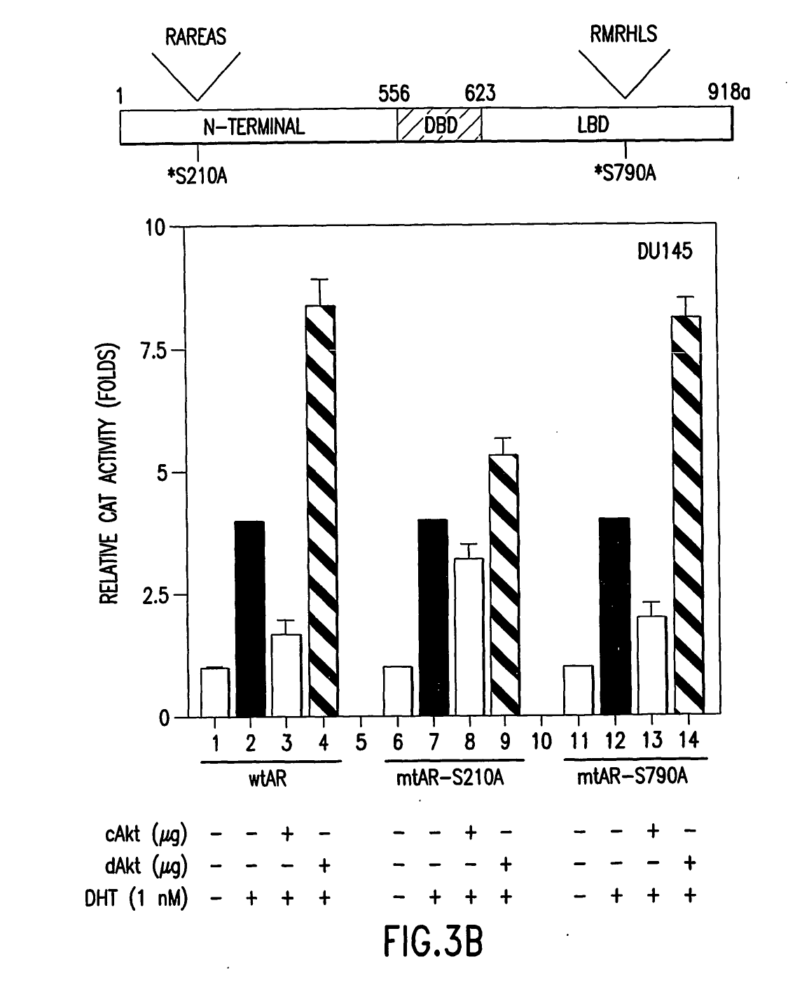

9. FIG. 3 shows AR is a downstream target of Akt. (a) Interaction of Akt with AR in vivo. Cells lysates were immunoprecipitated (IP) with anti-Akt or normal IgG (N-IgG). The immunoprecipitated complexes were immunoblotted (IB) with AR antibody (NH27) or anti-Akt antibody, respectively. (b) Transactivation by mtARS210A is not inhibited by Akt. DU145 cells were transfected with plasmids encoding wtAR, mtARS210A or mtARS790S in conjunction with cAkt or dAkt for 24 h. The ligand treatment and transactivation was determined as previously described.

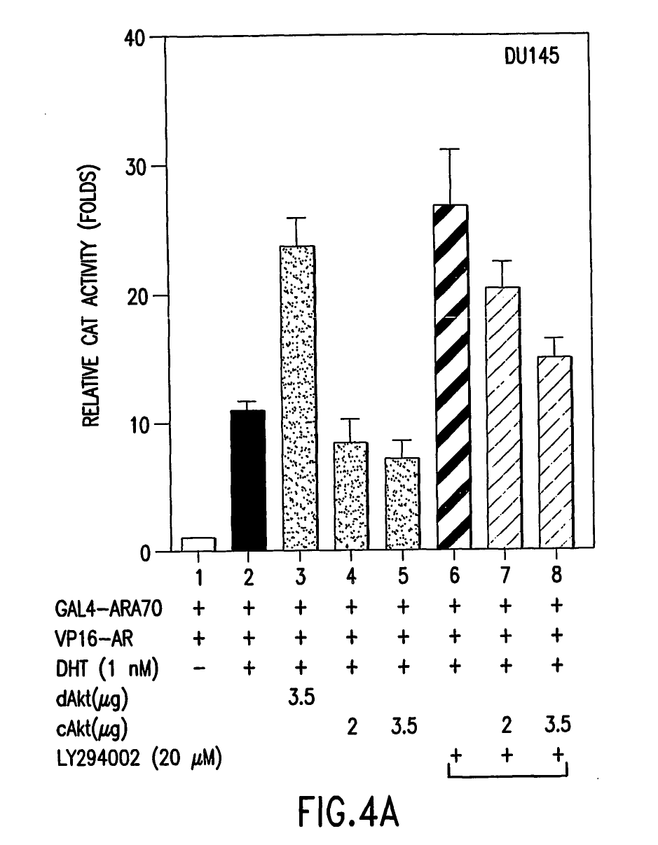

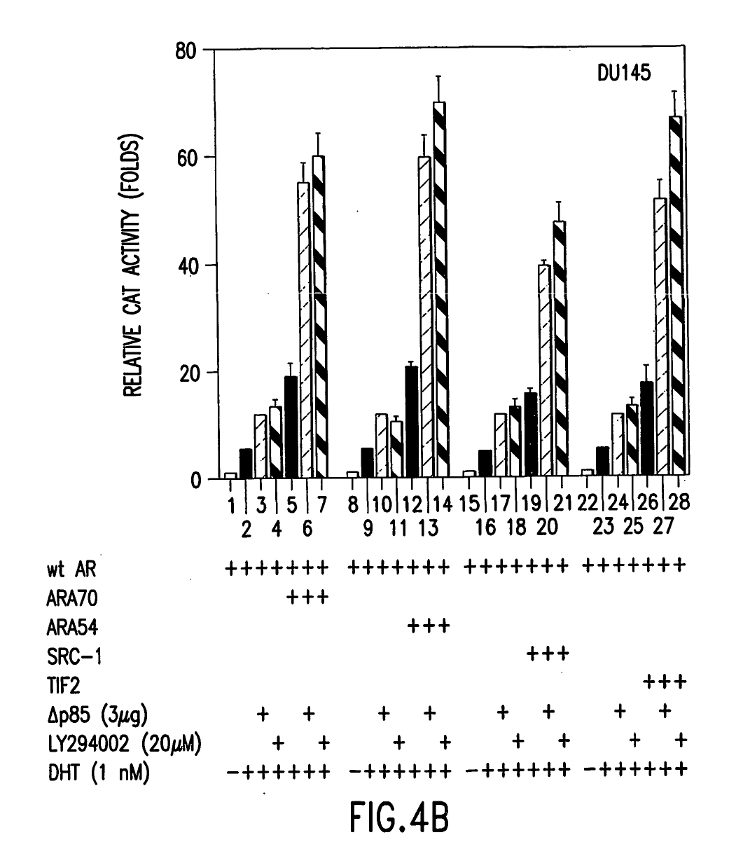

10. FIG. 4 shows the effect of PI3K on the interaction between AR and ARAs. (a) Modulation of interaction between AR and ARAs by cAKT, dAkt or LY294002. The DU145 cells were transfected with 2.5 μg GAL4-ARA70 and 2.5 μg VP16-AR, followed by treatment with LY294002 or vehicle 30 min prior to DHT treatment. The interaction between AR and ARA70 was determined by CAT assay using pG5CAT as reporter (b) The enhanced AR transactivation by various AR coactivators, such as ARA70, ARA54, TIF2 or SRC-1 could be further promoted in the presence of LY294002 or Δp85. The transactivation was determined as previously described.

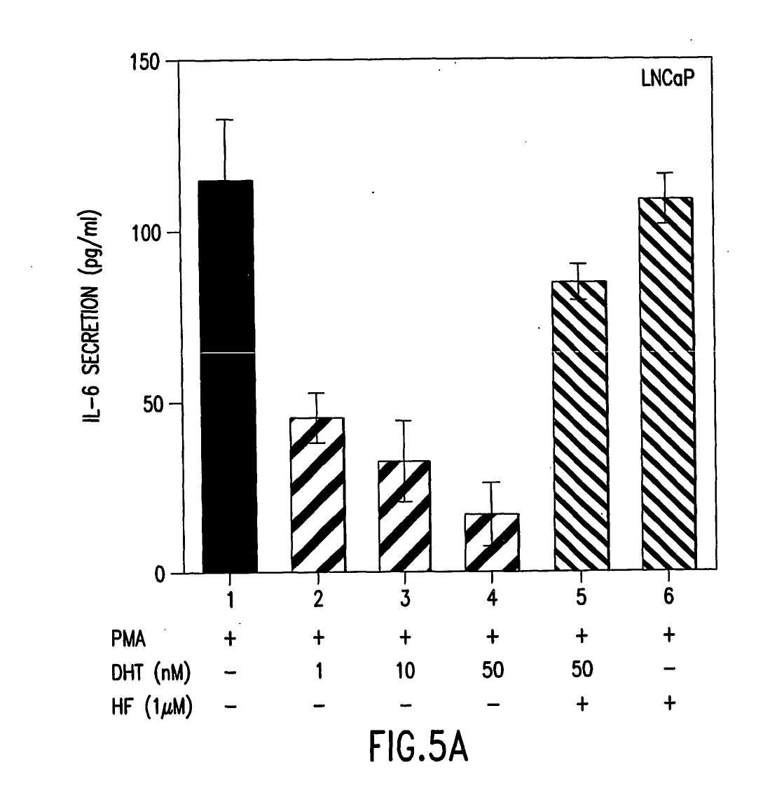

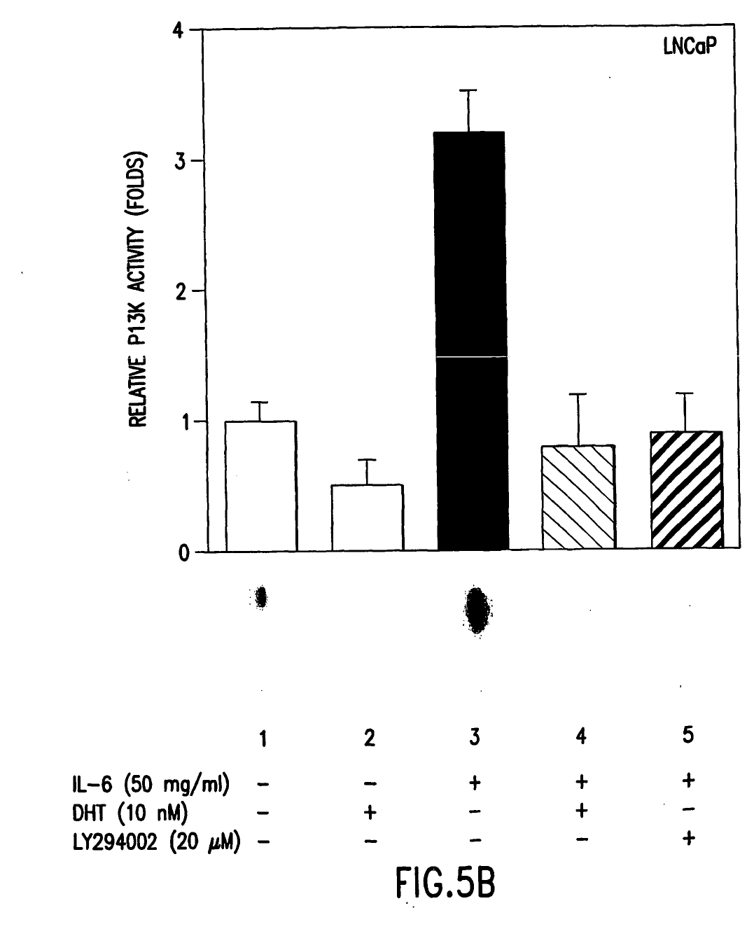

11. FIG. 5 shows the feed-back inhibition on IL-6 secretion and PI3K activity by A-AR. (a) Inhibition of IL-6 secretion by DHT. LNCaP cells were treated with DHT for 24 h and then PMA was added for another 24 h. IL-6 secretion in the medium was determined by ELISA. (b) Inhibition of IL-6-mediated PI3K activation by DHT. LNCaP cells, were serum starved for 24 h. DHT and IL-6 were added for 30 min. PI3K activity was measured using PI as a substrate. The origin (ori) of the chromatogram and PI3-phosphate (PI3P) were indicated.

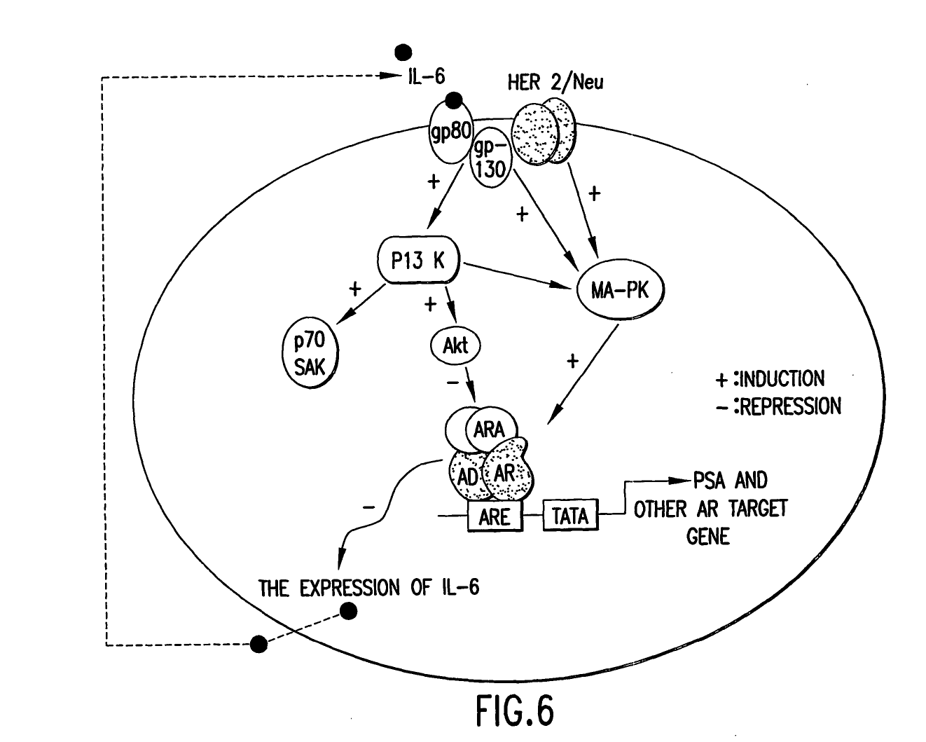

12. FIG. 6 shows a model for the cross-talk and bi-directional regulation of AR and IL-6 signal cascade. See text for details.

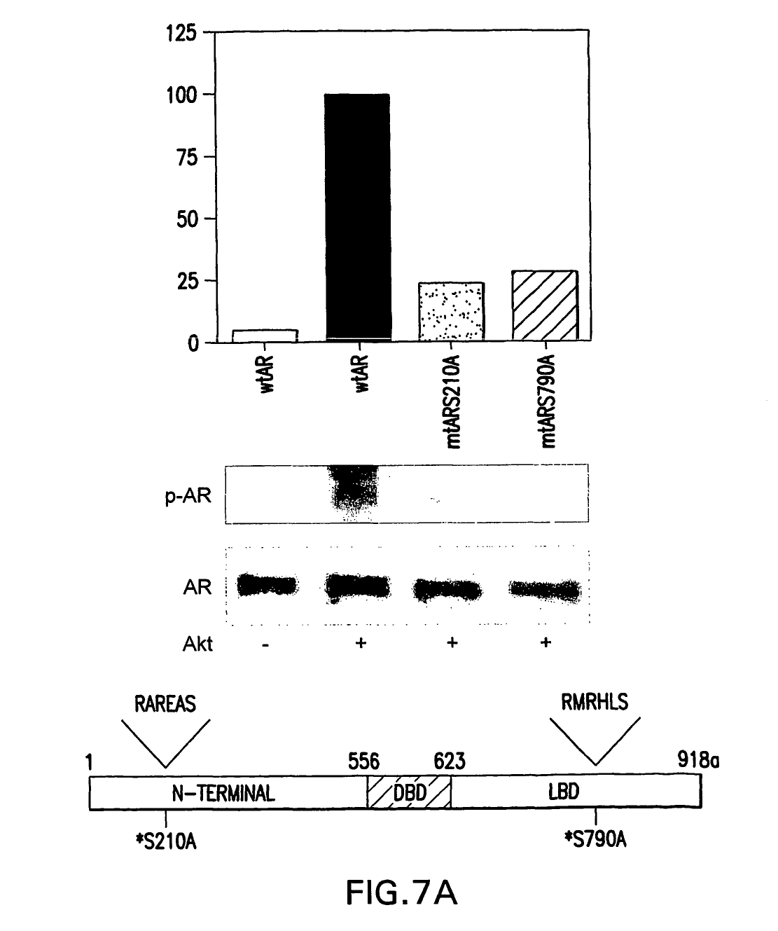

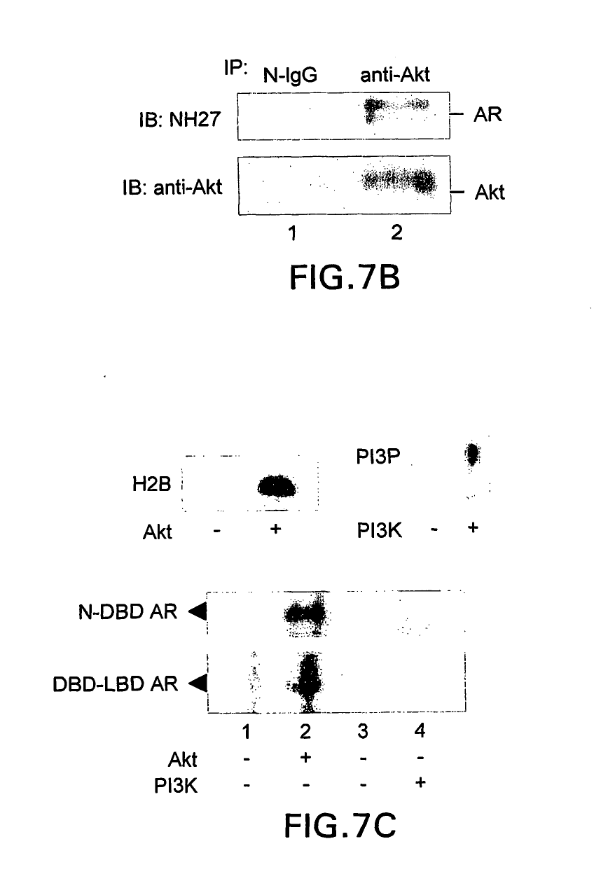

13. FIG. 7 shows AR is a direct Akt target (A) Akt-consensus phosphorylation sites (S210 and S790) of AR are responsible for AR phosphorylation. wtAR, mtAR S210A, or mtAR S790A were transfected into DU145. After transfection, whole cell extract was immunoprecipitated with the anti-AR antibody, NH27. Half of the precipitated complex was treated with Akt and [γ-³²P] ATP for 2 h and analysed by SDS-PAGE. In order to verify the equal expression levels of the wtAR and mtAR constructs, the remaining immunoprecipitates were subjected to western blot analysis as shown on the bottom panel. (B) Akt interacts with AR in LNCaP cells in vivo. LNCaP cell lysates were immunoprecipitated (IP) with anti-Akt or normal IgG (N-IgG). The immunoprecipitated complexes were immunoblotted (IB) with AR antibody (NH27) or anti-Akt antibody, respectively. (C) In vitro phosphorylation of AR by Akt, but not by PI(3)K. 1 μg N-DBD AR or 1 μg DBD-LBD AR purified from E. coli. was treated for 1 h with Akt or PI(3)K. Phosphorylation of the AR was detected by separation on 12.5% SDS-PAGE and autoradiography. The Akt and PI(3)K used in this experiment are active, as determined by phosphorylating H2B and PI, respectively, which is shown on the upper panel of FIG. 7A.

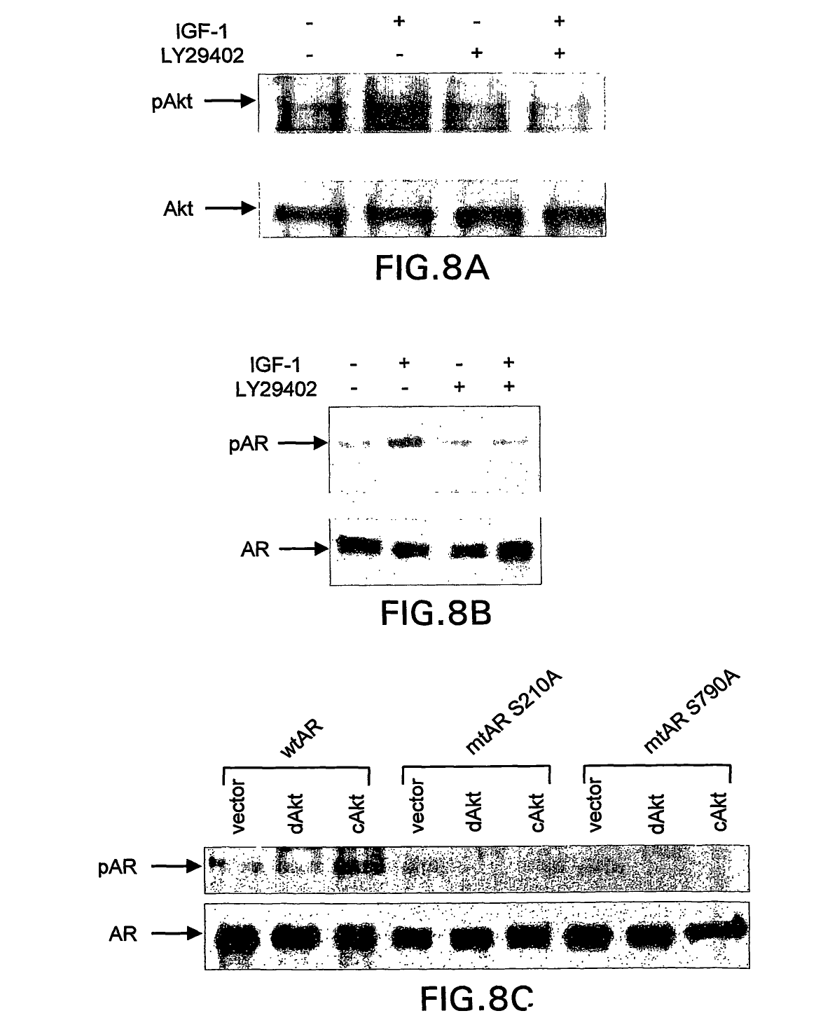

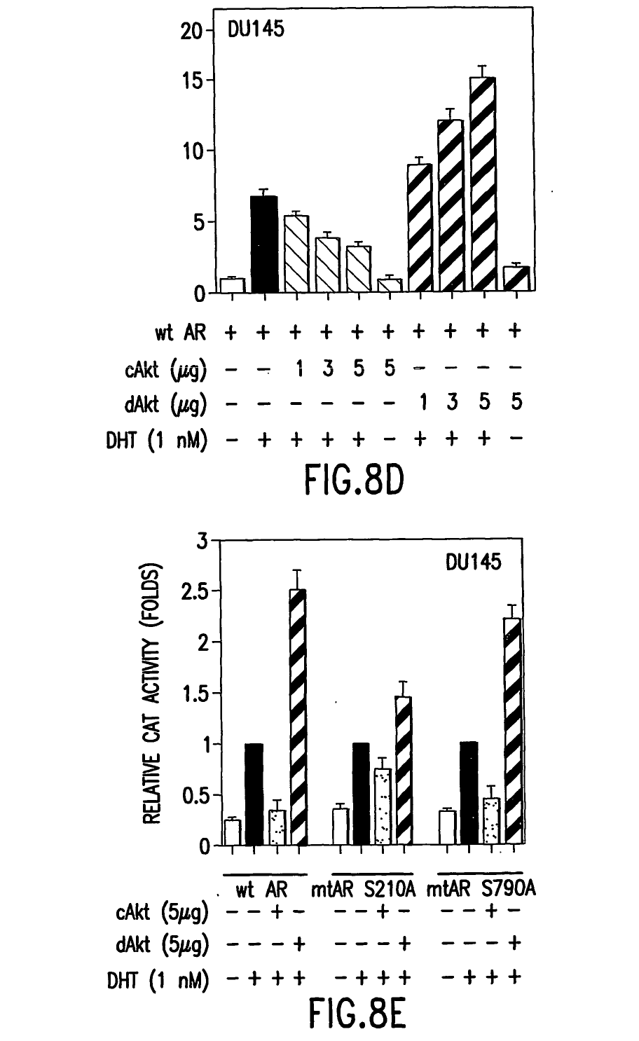

14. FIG. 8 shows phosphorylation of AR by Akt in vivo. (A) Activation of Akt by IGF-1. COS-1 cells were pretreated with ethanol or 20 μM LY294002 for 30 min, followed by treatment with 100 ng/ml IGF-1 for 30 min. The total cell lysates were immunoprecipitated by anti-Akt antibody (New England Biolabs). The immunocomplex was subjected to SDS-PAGE, followed by immunoblot with phospho-Akt (S473) antibody or Akt antibody. (B) IGF-1 phosphorylates AR in vivo via Akt. COS-1 cells were cultured in [³²P]-PO₄-containing medium. AR immunocomplex was subjected to SDS-PAGE followed by autoradiography. Immunobloting confirmed equivalent amounts of AR in immunocomplex. (C) cAkt, but not dAkt, phosphorylates wtAR, but not mtAR S210A and mtAR S790A in vivo. COS-1 cells were transfected with wtAR, mtAR S210A, or mtAR S790A in combination with PCDNA3 vector, cAkt, or dAkt. After 24 h transfection, cells were labeled with [³²P]-PO₄ for 4 h. (D) Suppression of AR transactivation by Akt in a dose-dependent manner. The DU145 cells were transfected with various plasmids, as indicated, for 24 h, followed by DHT treatment for another 24 h. Transactivation was measured by CAT activity using MMTV-CAT as a reporter. (E) mtAR S210A is resistant to Akt suppressive effect on AR transactivation. DU145 cells were transfected with plasmids encoding wtAR, mtAR S210A or mtAR S790A in presence of cAkt or dAkt for 24 h. Ligand treatment and AR transactivation were performed as previously described. The data are means±s.d. from three independent experiments.

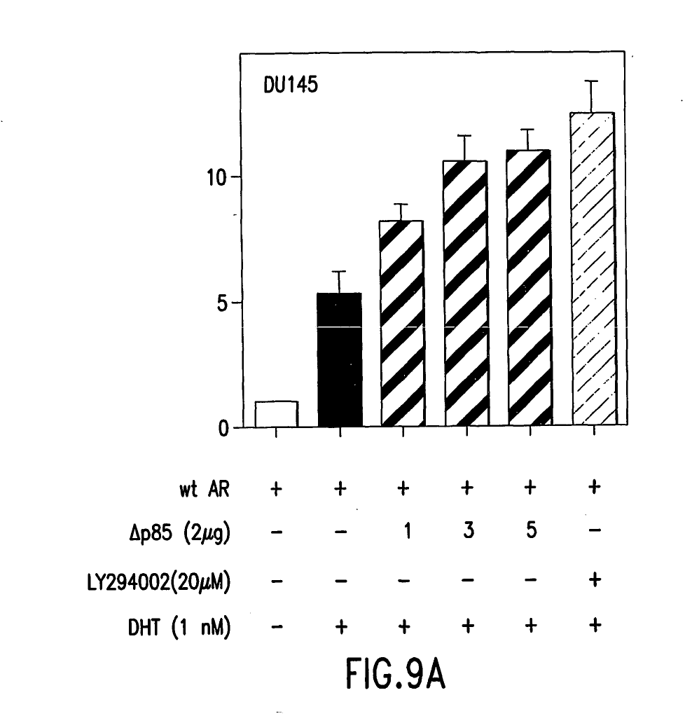

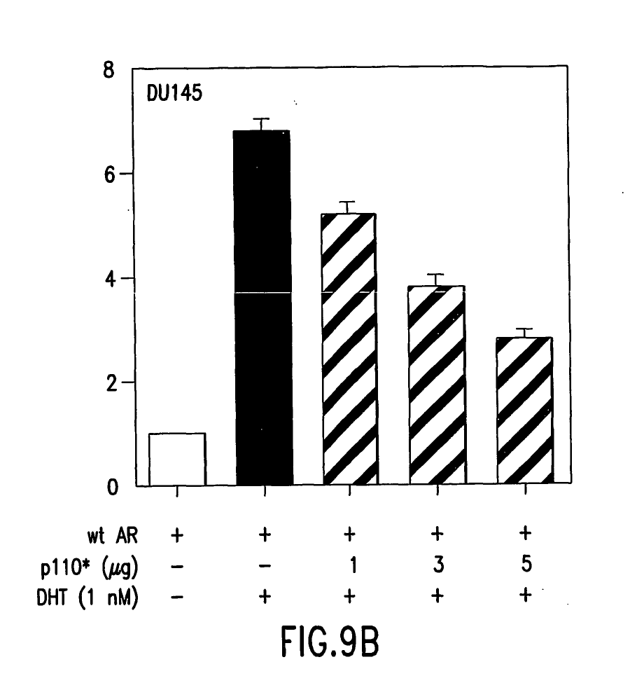

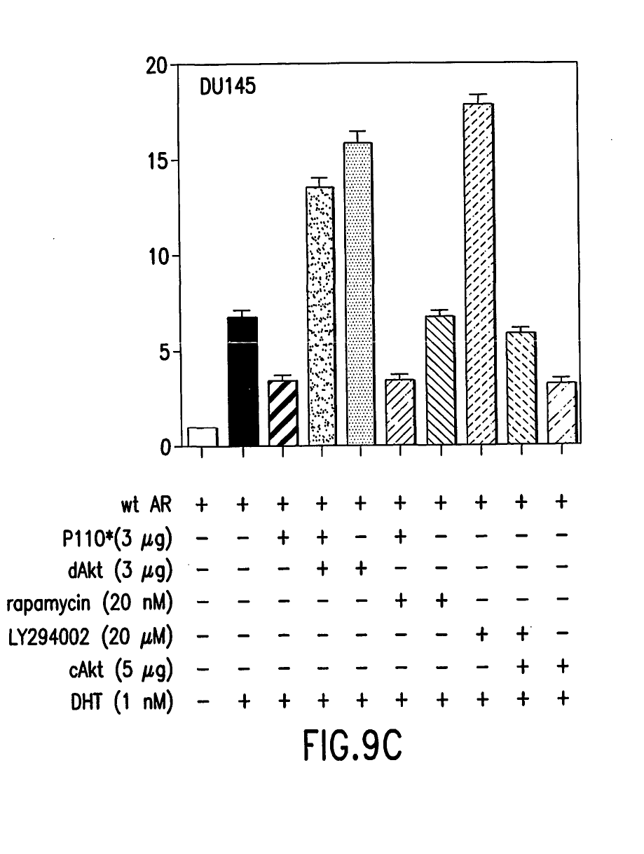

15. FIG. 9 shows inhibition of AR transactivation by PI(3)K/Akt pathway. (A) AR transactivation is enhanced through the inhibition of PI(3)K activity by Δp85 and LY294002. The DU145 cells were treated with LY294002 for 30 min prior to DHT treatment, the transactivation activity was determined after 24 h transfection. (B) Suppression of AR transactivation by p110* in a dose-dependent manner. (C) Suppression of AR transactivation by PI(3)K via Akt but not via p70S6K. The DU145 cells were transfected with plasmids, as indicated, for 16 h. Cells were treated with 20 nM rapamycin or 20 μM LY294002 for 30 min prior to 1 nM DHT treatment. The data are means±s.d. from three independent experiments.

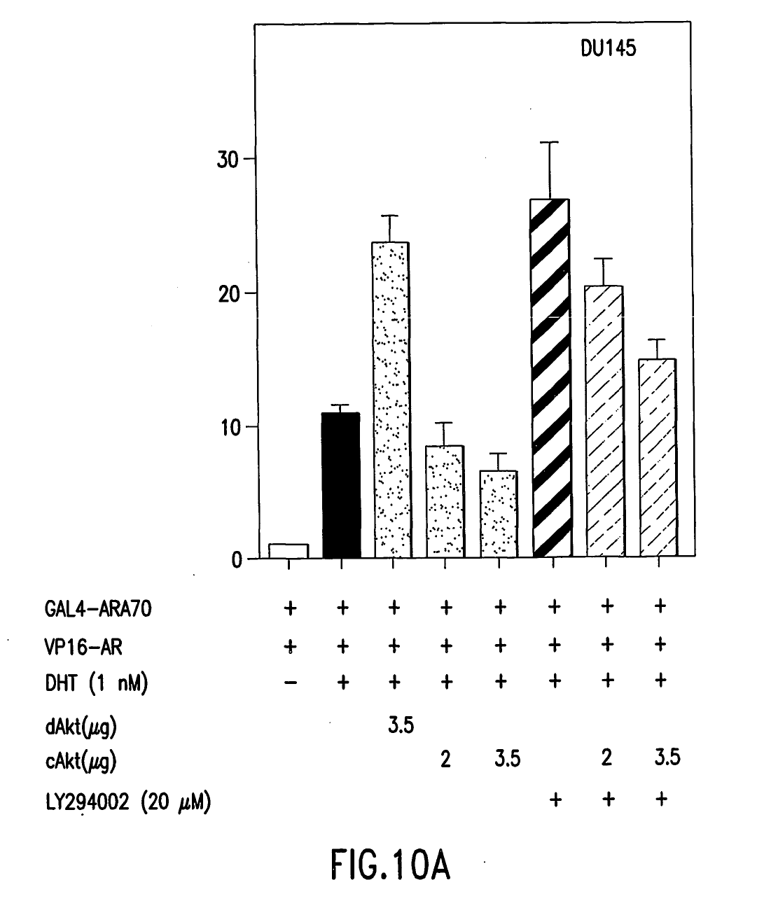

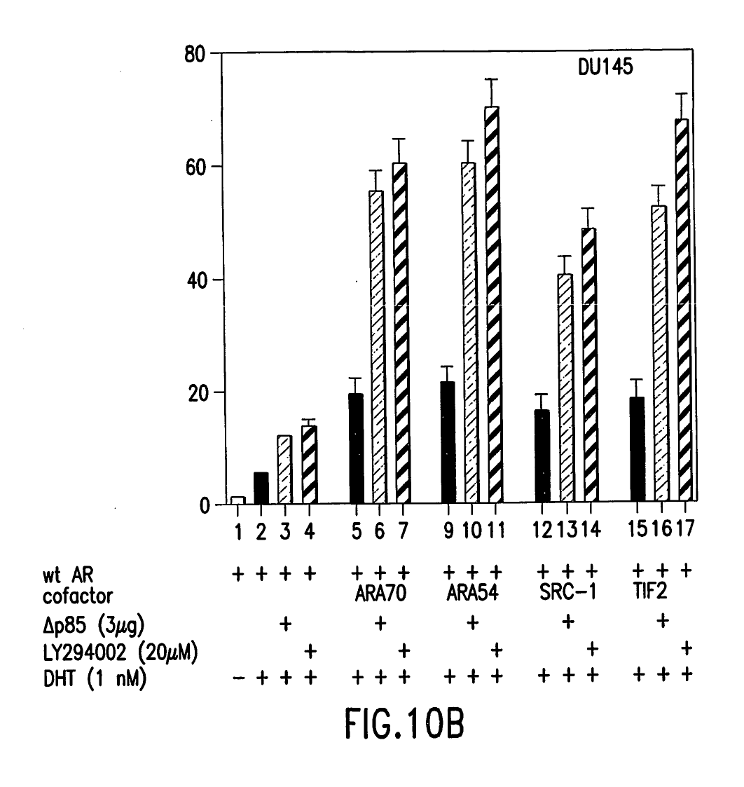

16. FIG. 10 shows effect of PI(3)K/Akt pathway on the interaction between AR and ARA70. (A) Modulation of interaction between AR and ARA70 by cAkt, dAkt, or LY294002. The DU145 cells were transfected with 2.5 μg GAL4-ARA70 and 2.5 μg VP16-AR, followed by treatment with LY294002 or vehicle 30 min prior to DHT treatment. The interaction between AR and ARA70 was determined by CAT assay using pG5-CAT as a reporter. (B) The enhanced AR transactivation by various AR coactivators, ARA70, ARA54, TIF2, and SRC-1, could be further promoted in the presence of LY294002 or Δp85. The data are means±s.d. from three independent experiments.

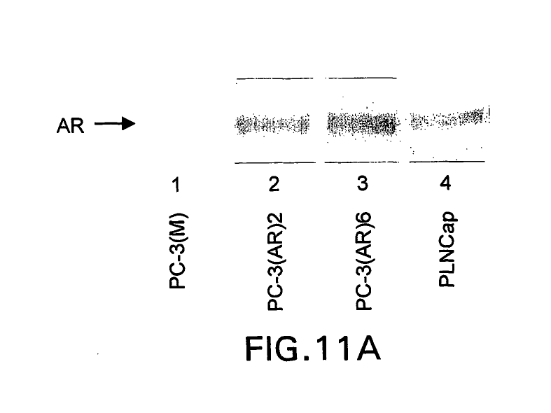

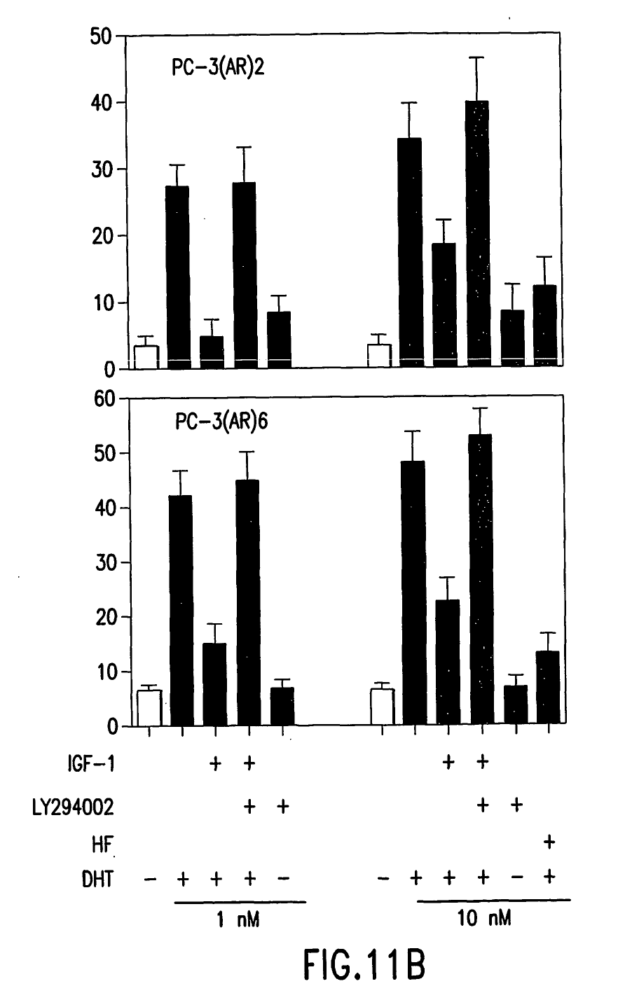

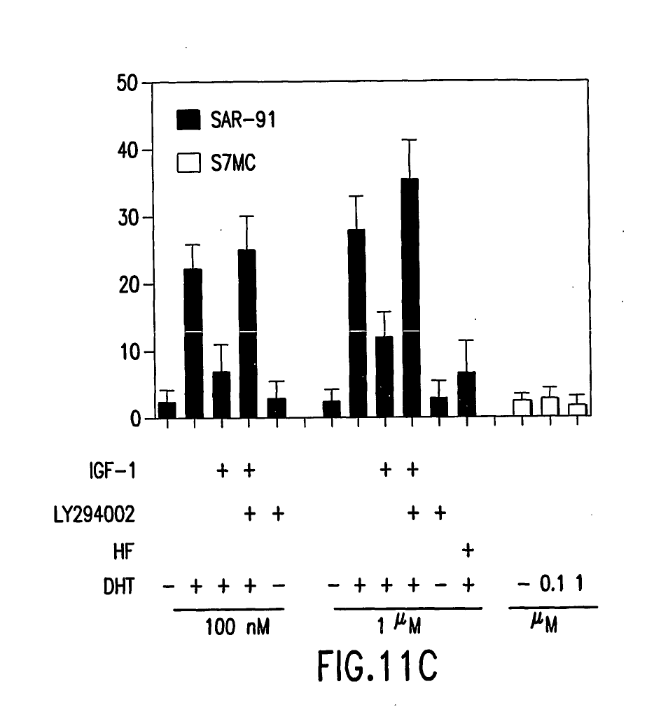

17. FIG. 11 shows PI(3)K/Akt pathway suppressed androgen/AR-induced apoptosis. (A) PC-3(AR)2 and PC-3(AR)6 expressed AR protein. PC-3 cells were stably transfected with AR, followed by selection with hygromycin B, and confirmed by western blotting using AR antibody NH27, while LNCaP was used as a positive control. (B) Androgen/AR-induced apoptosis in PC-3(AR)2, PC-3(AR)6, and SAR-91 were inhibited by PI(3)K/Akt pathway. SAR-91, S7MC, PC-3(AR)2, and PC-3(AR)6 were treated with LY294002 (20 μM) or HF (5 μM) for 30 min, followed by addition of IGF-1 (100 ng/ml) for another 30 min prior to DHT (10 nM) treatment After 3 days, cell apoptosis was analyzed by TUNEL assay. The data are means±s.d. from three independent experiments.

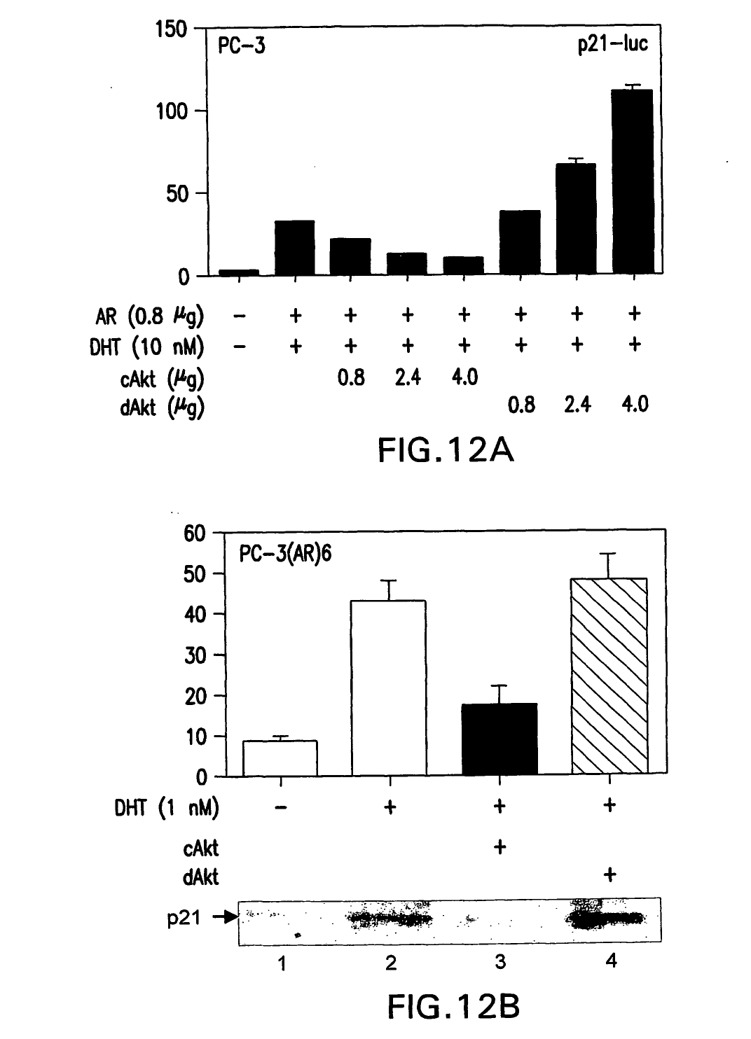

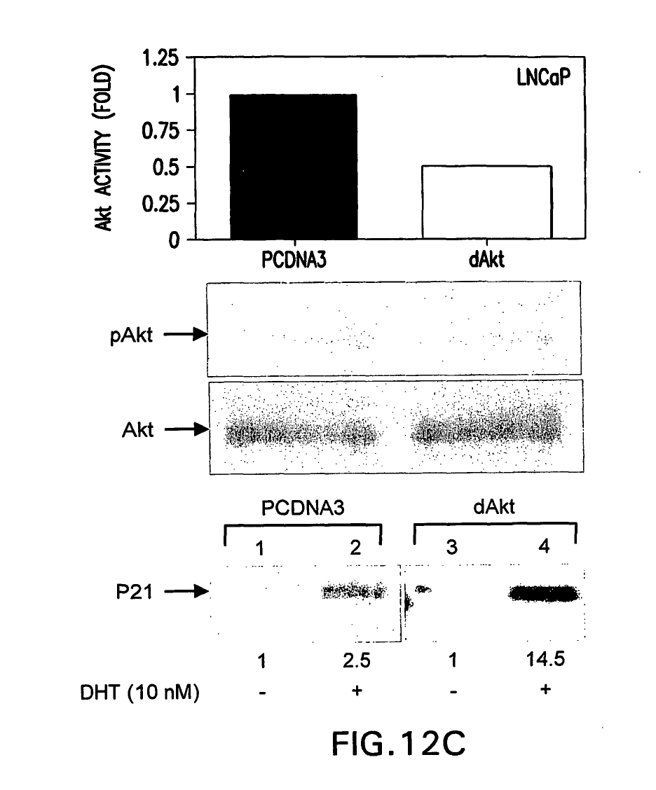

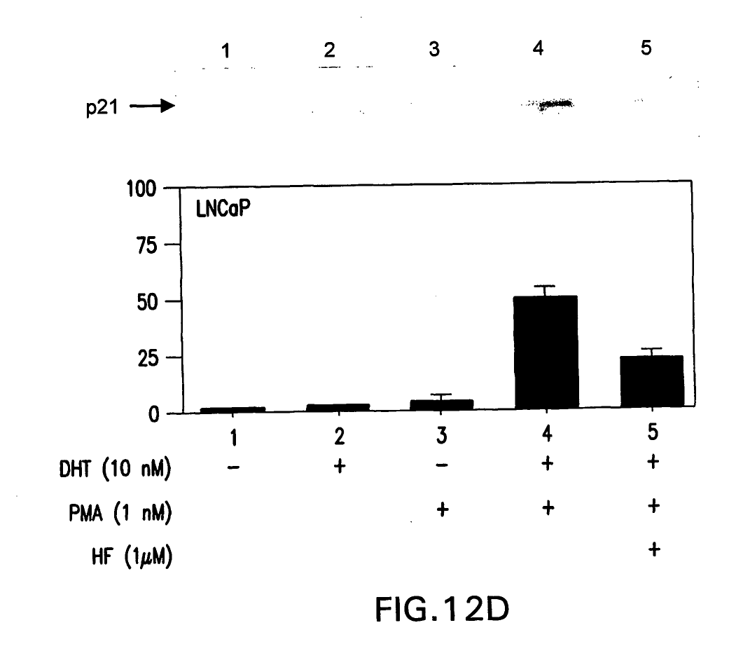

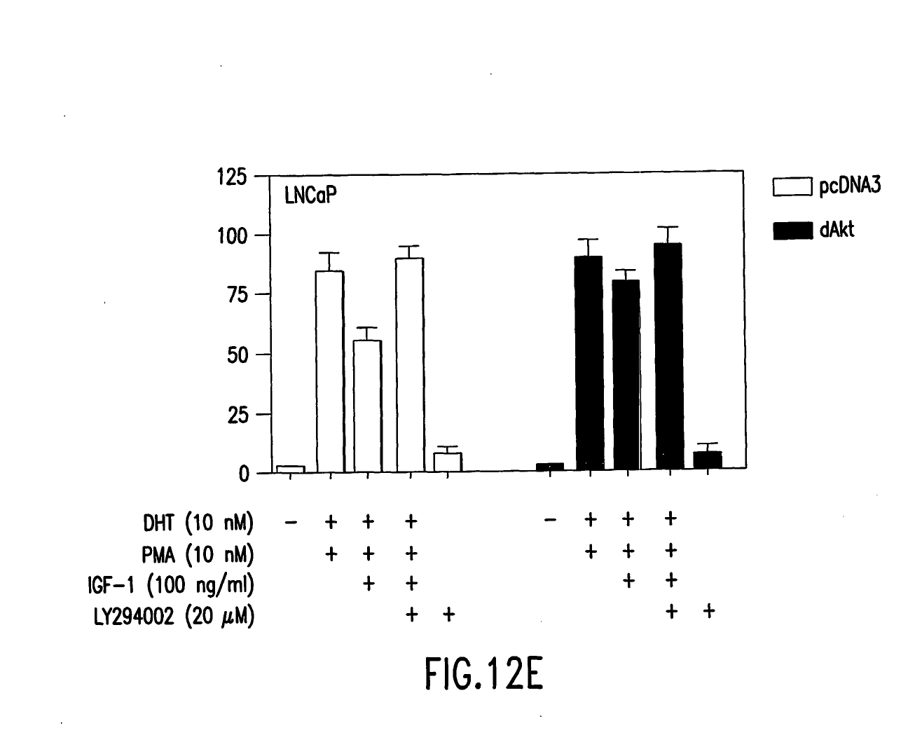

18. FIG. 12 shows androgen/AR-induced apoptosis and p21 expression were inhibited by Akt. (A) Akt suppressed androgen/AR-induced p21 promoter activity. PC-3 cells were transfected with different plasmids, as indicated, for 16 h, followed by DHT treatment for another 16 h. p21 promoter activity was determined by luciferase activity. (B) Androgen/AR-induced PC-3(AR)6 apoptosis and p21 protein expression was blocked by Akt PC-3(AR)6 was transfected with pCDNA3, cAkt, or dAkt, as indicated, for 16 h. The cells were then treated with DHT for 3 days, then cell apoptosis determined by TUNEL assay. p21 protein expression was detected by western blotting using p21 monoclonal antibody. (C) AR-induced p21 protein expression was enhanced by dAkt in LNCaP cells. LNCaP stable clones (pCDNA3 and dAkt) were treated with 10 nM DHT for 2 days, then the p21 protein expression was detected. LNCaP stable transfection with dAkt was confirmed by western blot assay using phospho-Akt (Ser473) antibody. (D) Potentiation of DHT/TPA-induced cell apoptosis and p21 protein expression by androgen in LNCaP cells. LNCaP cells were pretreated with HF or vehicle for 30 min followed by treatment with DHT for 24 h. TPA was then added for another 24 h and cell apoptosis was determined by TUNEL assay. (E) Activation of PI(3)K/Akt pathway by IGF-1 suppresses DHT/TPA-induced apoptosis. LNCaP stable clones (pCDNA3 and dAkt) were treated with 10 nM DHT for 24 h followed by treatment with 20 μM LY294002 for 30 min. IGF-1 was added for another 30 min, followed by 10 nM TPA treatment for another 24 h. The apoptosis was determined by TUNEL assay. The data are means±s.d. from three independent experiments.

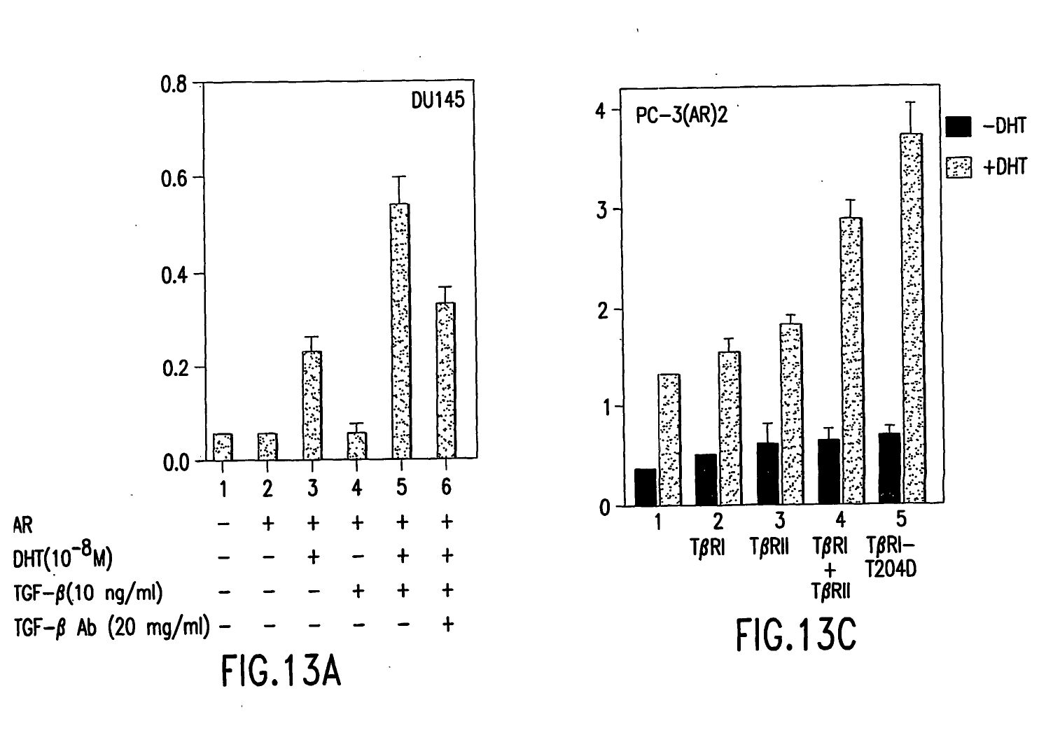

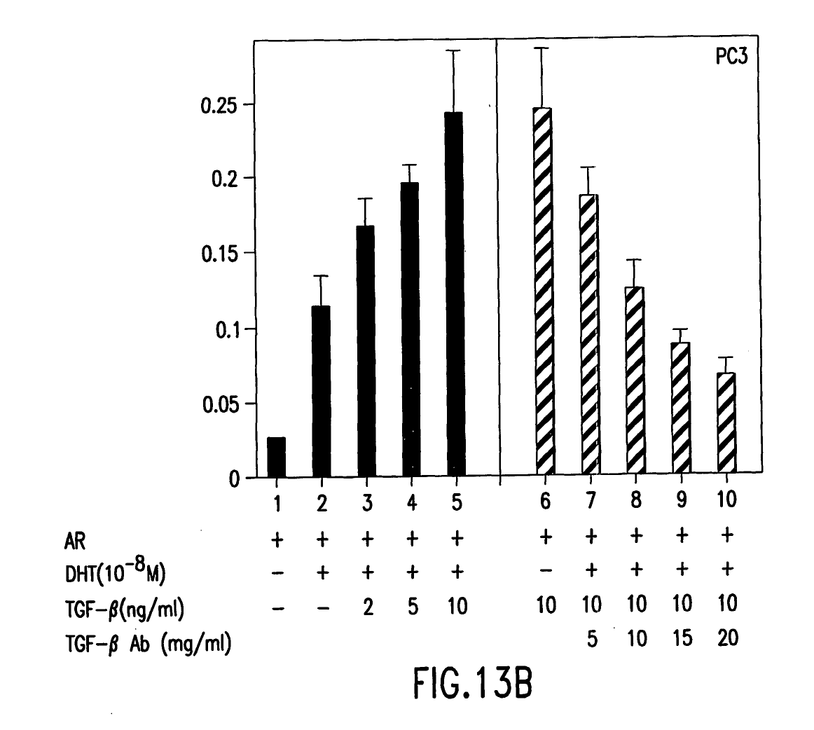

19. FIG. 13 shows the ligand-induced transactivation of AR is enhanced by treatment with TGF-β. (A) CAT assays were performed with extracts from DU145 cells transfected with AR expression vector (pSG5-AR) (1 μg) in the presence (+) or absence (−) of DHT (10⁻⁸ M) or TGF-β1 (10 ng/ml) or specific TGF-β1 neutralizing antibody (20 mg/ml). (B). In the left panel, PC-3 cells were transfected with pSG5-AR (1 μg) in the presence (+) or absence (−) of DHT (10⁻⁸ M) with increasing amounts of TGF-β1. In the right panel, a fixed amount of TGF-β1 (10 ng/ml) was added into transfected PC-3 cells with increasing amounts of specific TGF-β1 neutralizing antibody. (C), PC-3(AR)2 cells stably transfected with AR were overexpressed with TGF-β type I (TβRI) or type II (TβRII)receptor or constitutively active TGF-β type I receptor (TβRI-T204D) as indicated. 3 μg of MMTV-CAT or MMTV-Luc was used as a reporter plasmid in all experiments. All values represent the averages+SD of four independent experiments.

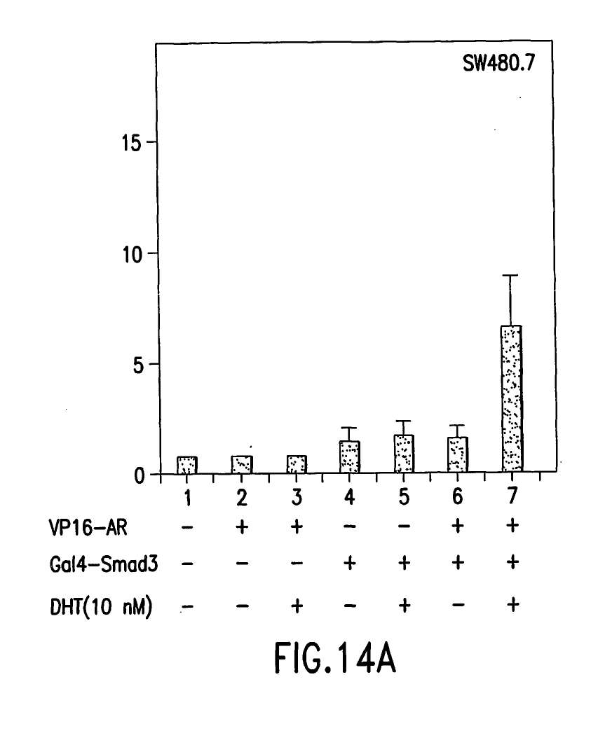

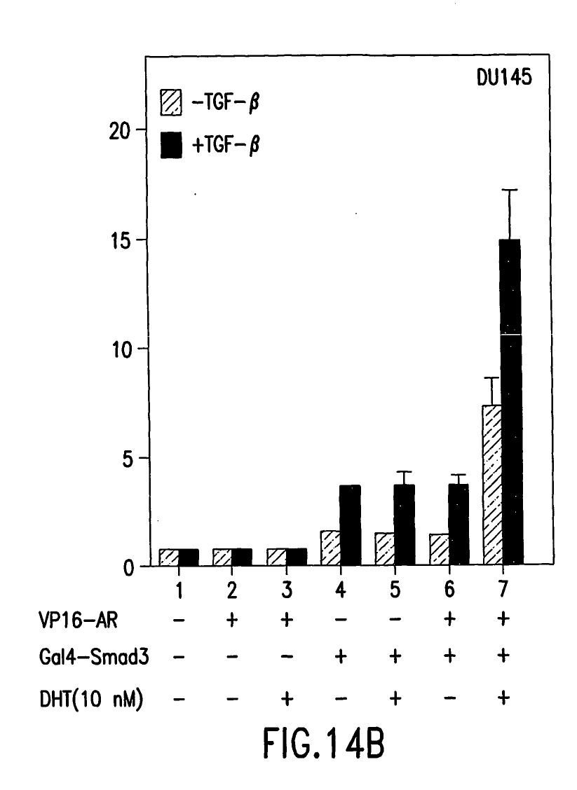

20. FIG. 14 shows the association of Smad3 with AR in mammalian two-hybrid interaction system. (A) SW480.7 cells were co-transfected with 3 μg of Gal4-Smad3 encoding the full-length cDNA of Smad3 fused to the Gal4-DBD and 4.5 μg of VP16-AR encoding the full-length cDNA of AR fused to the activation domain of VP16. Interaction was estimated by determining the level of CAT activity from 3 μg of the reporter plasmid pG5-CAT in the presence of 10⁻⁸ M DHT. (B) DU145 cells were transfected with Gal4-Smad3 and VP16-AR expression-vectors in the presence (+) or absence (−) of DHT and TGF-β. Each CAT activity is presented relative to the transactivation observed in the absence of DHT. All values represent the mean+SD of four independent experiments.

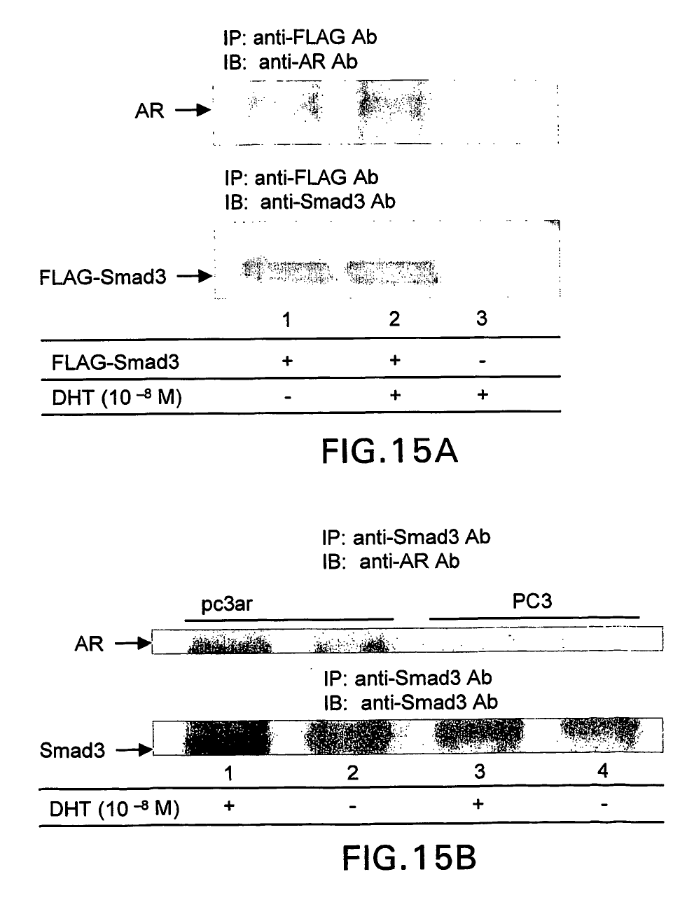

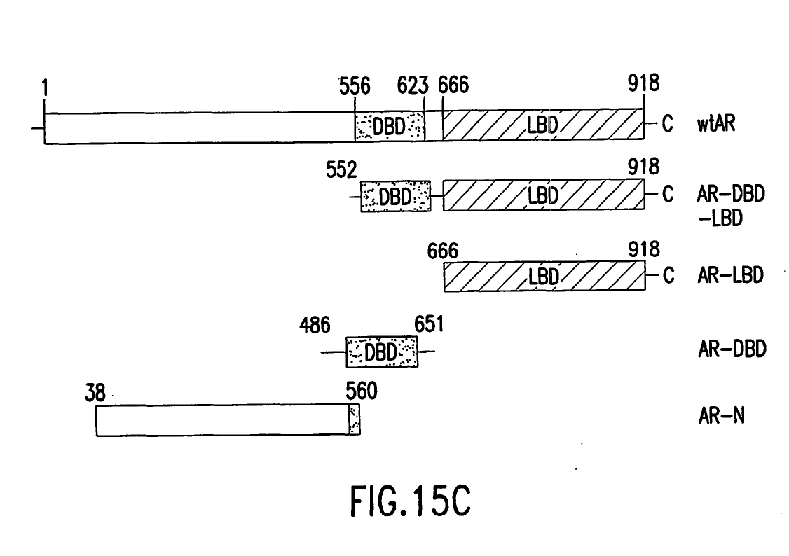

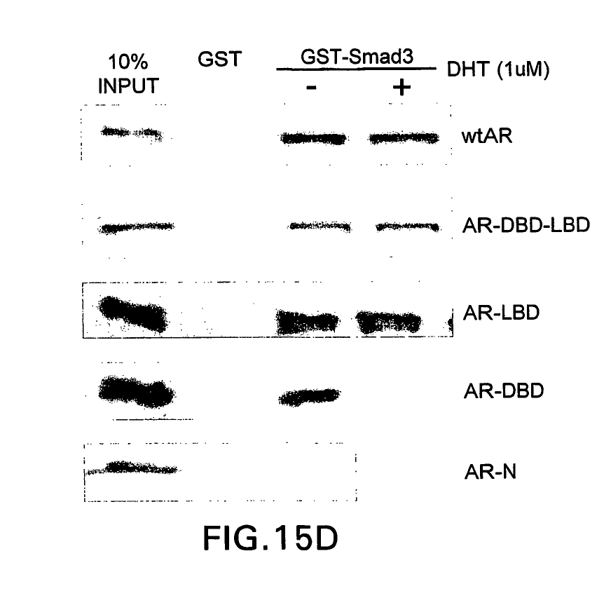

21. FIG. 15 shows In vivo and in vitro interaction between Smads and AR. (A) and (B) Co-immunoprecipitation of AR and Smad3. (A) PC-3 cells that overexpressed Flag-Smad3 and AR (B) PC-3 and PC-3(AR)2 cells were treated with or without DHT. Cell extracts were prepared and immunoprecipitations were performed using anti-FLAG antibody or anti-Smad3 antibody, followed by immunoblotting using antibody to AR. (C) The wtAR and different AR deletion mutants used in the GST-pull down assay are show schematically. (D) Interaction domains of AR for Smad3. A series of [³⁵S]-labeled mtARs incubated with GST-Smad3 or GST alone in the presence (+) or absence (−) of 10 nM DHT were tested for interaction in the GST pull-down assay.

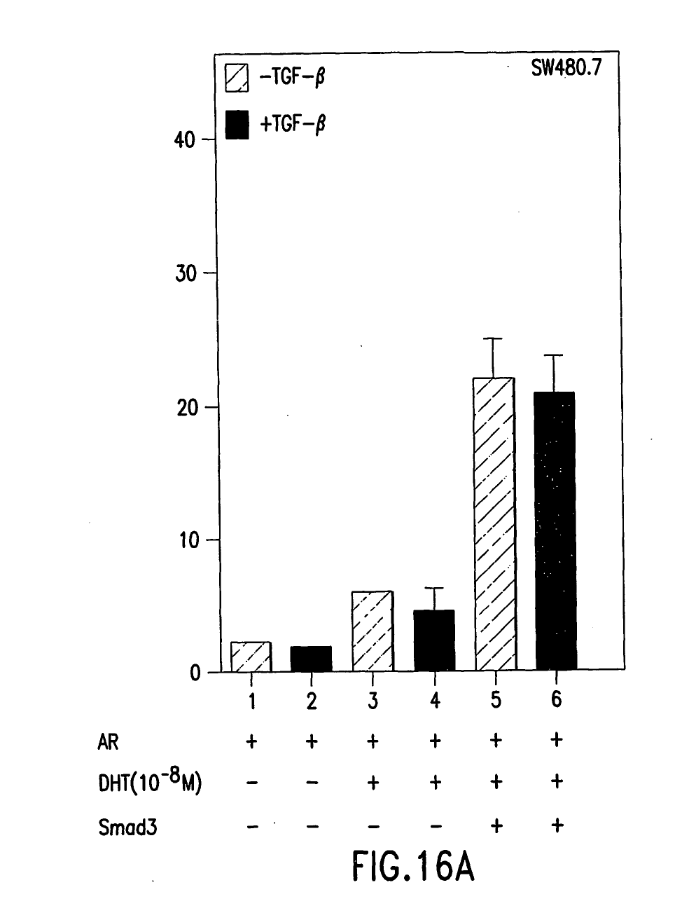

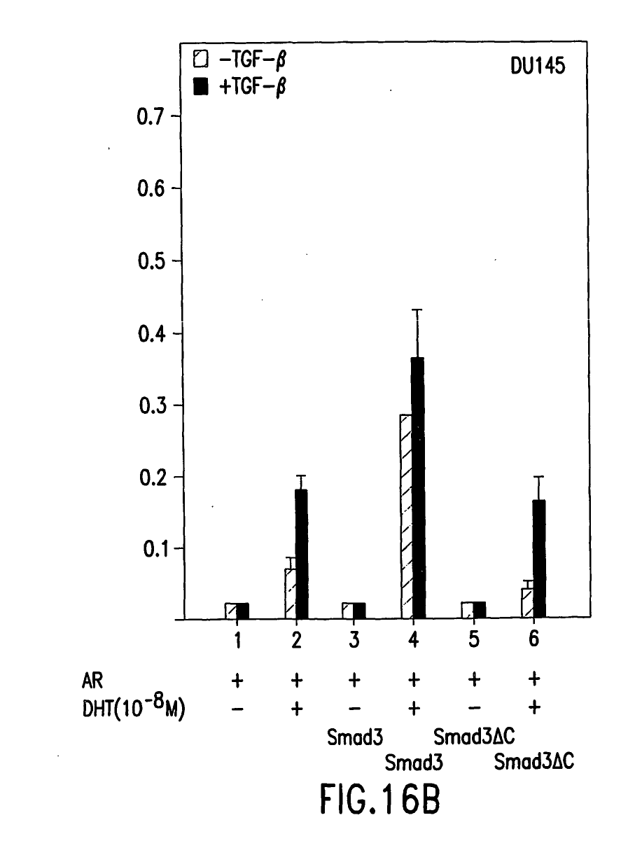

22. FIG. 16 shows the effects of Smad3 on AR-mediated transcriptional activity. (A) SW480.7 cells were co-transfected with 1 μg of pSG5-AR, 3 μg of MMTV-CAT, and 3 μg of Smad3 expression vectors in the presence (+) or absence (−) of DHT (10⁻⁸ M) or TGF-β (10 ng/ml). (B) DU145 cells were co-transfected with 3 μg of Smad3 or Smad3ΔC mutant expression vectors with 1 μg of pSG5-AR and 3 μg of MMTV-CAT, in the presence (+) or absence (−) of DHT (10⁻⁸ M) or TGF-β (10 ng/ml). Each CAT activity is presented relative to the transactivation observed in the absence of DHT and an error bar represents the mean+SD of four independent experiments.

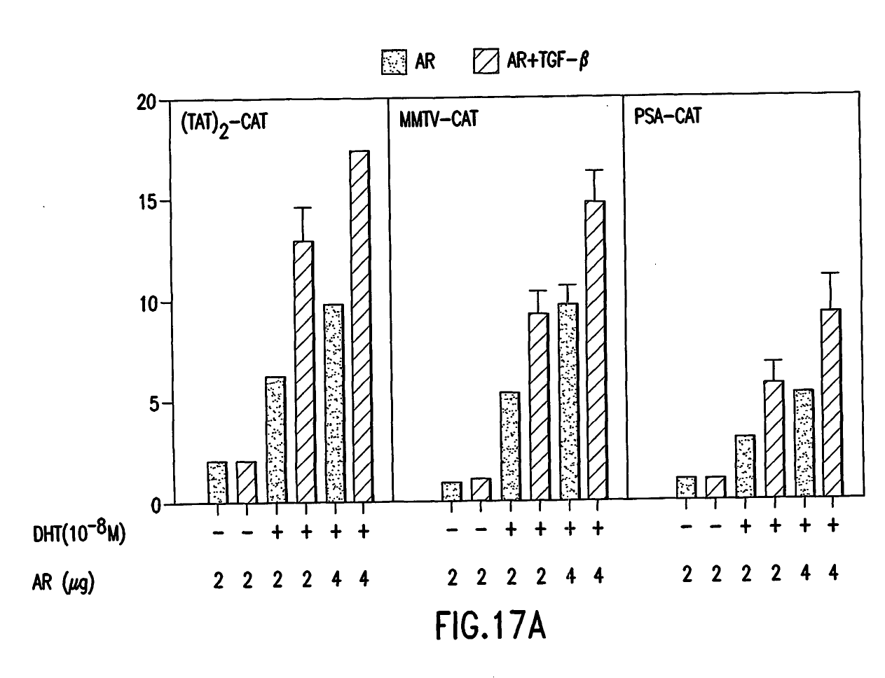

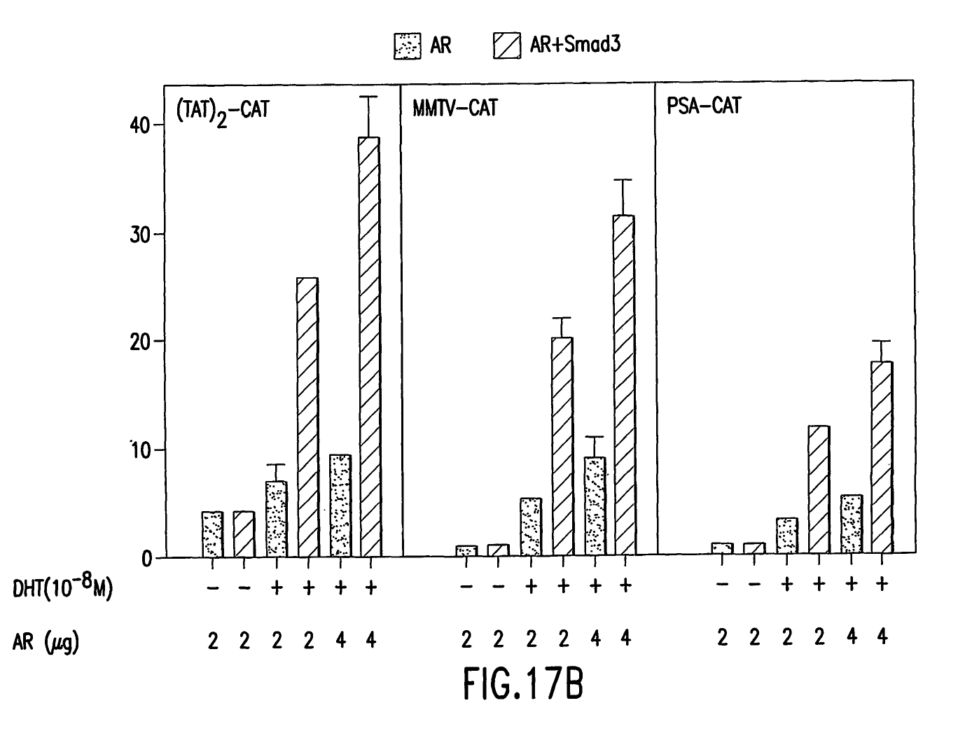

23. FIG. 17 shows the androgen-response element is important for TGF-β/Smad3-enhanced AR transactivation. (A) DU145 cells were transiently co-transfected with AR (2 or 4 μg) and either (TAT)₂-CAT, MMTV-CAT or PSA-CAT (3 μg), in the presence (+) or absence (−) of DHT (10⁻⁸ M) or TGF-β(10 ng/ml). (B) DU145 cells were transiently co-transfected with AR (2 or 4 μg) and either (TAT)₂-CAT, MMTV-CAT or PSA-CAT (3 μg), and Smad3 expression vector (6 or 10 μg) in the presence (+) or absence (−) of DHT (10⁻⁸ M). Each CAT activity is presented relative to the transactivation observed in the absence of Smad3. All values represent the mean+SD of three independent experiments.

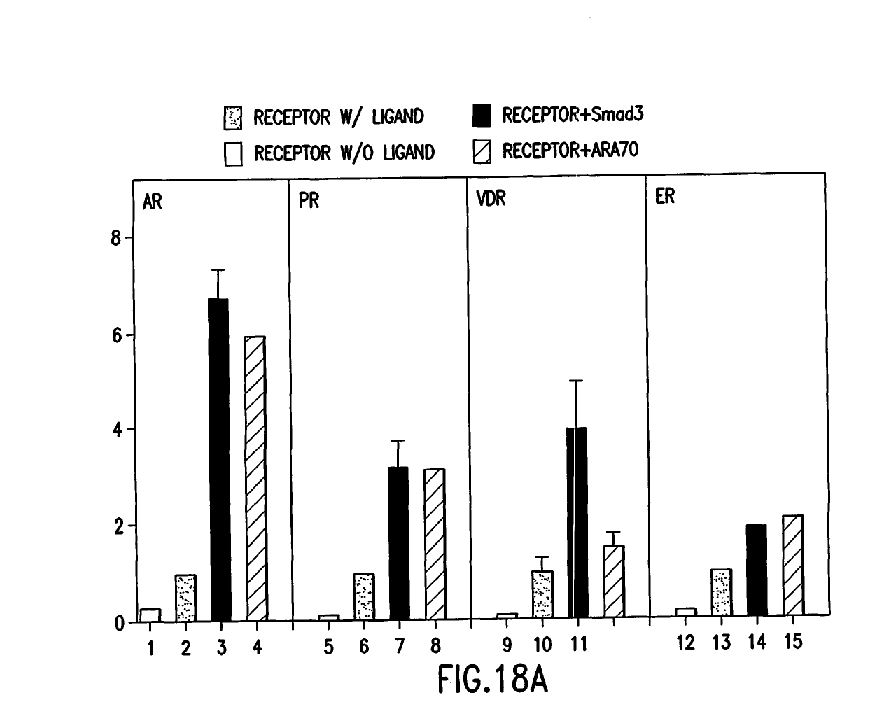

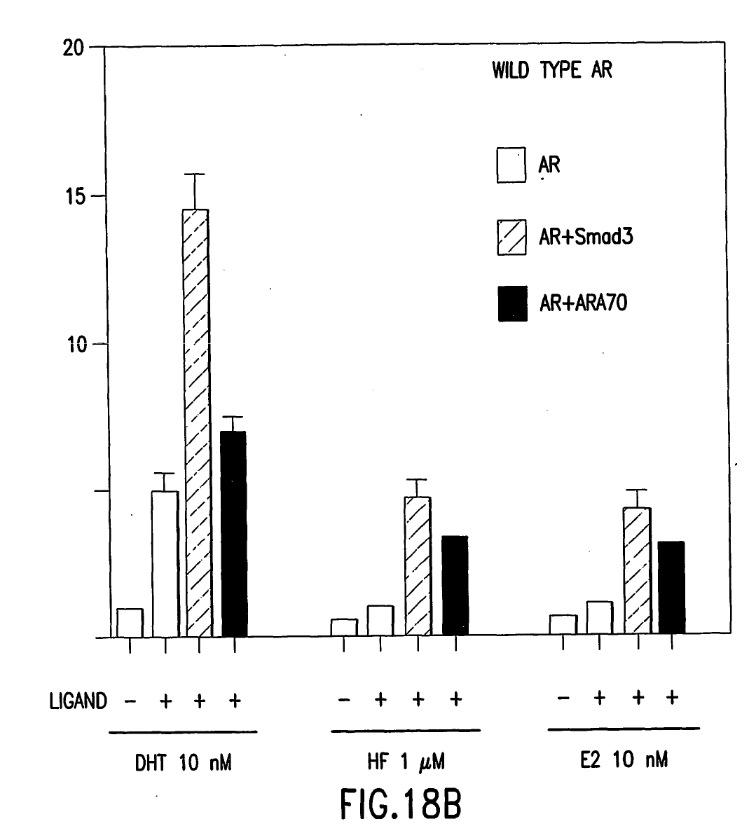

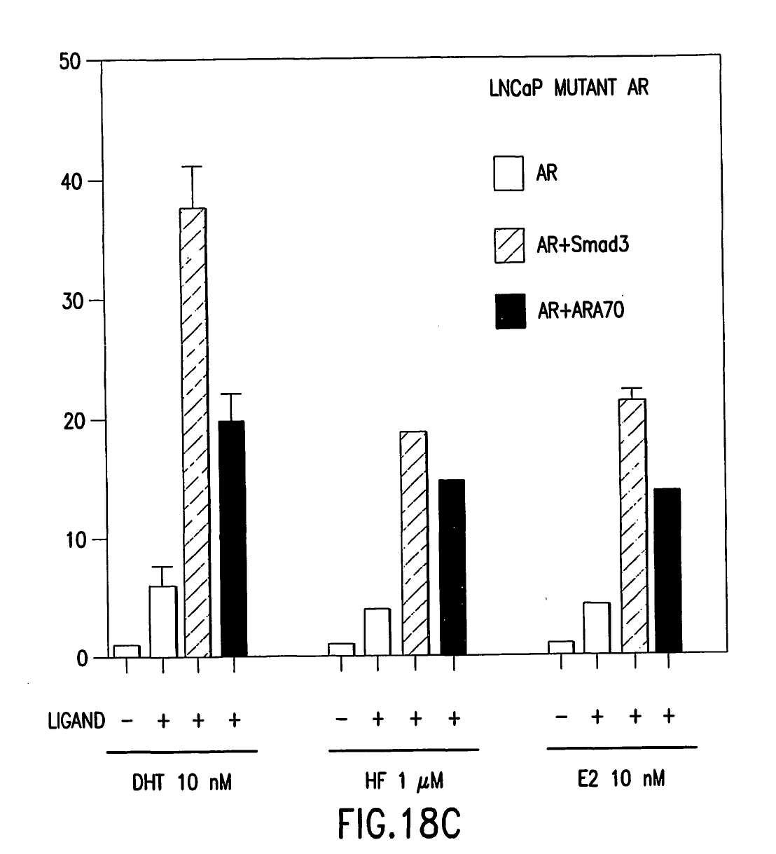

24. FIG. 18 shows the effect of Smad3 on the transcriptional activities of wtAR, mtAR, PR, VDR, and ER. (A) DU145 cells were transiently co-transfected with 3 μg of reporter plasmids (MMTV-CAT for AR, and PR, ERE-CAT for ER and VDRE-CAT for VDR), 1 μg of each receptor constructed in pSG5, and 4.5 μg of Smad3 expression vector in the presence of 10⁻⁸ M of each cognate ligand. Each luciferase and CAT activity is presented relative to the transactivation observed in the absence of Smad3. (B) 1.5 μg of wtAR were co-transfected with 4.5 μg of Smad3 or ARA70 in the absence or presence of DHT, E2, or HF at indicated concentrations. (C) The LNCaP mtARt877a was used to replace the wtAR to perform the same experiment as (B). All values represent the mean+SD of three independent experiments.

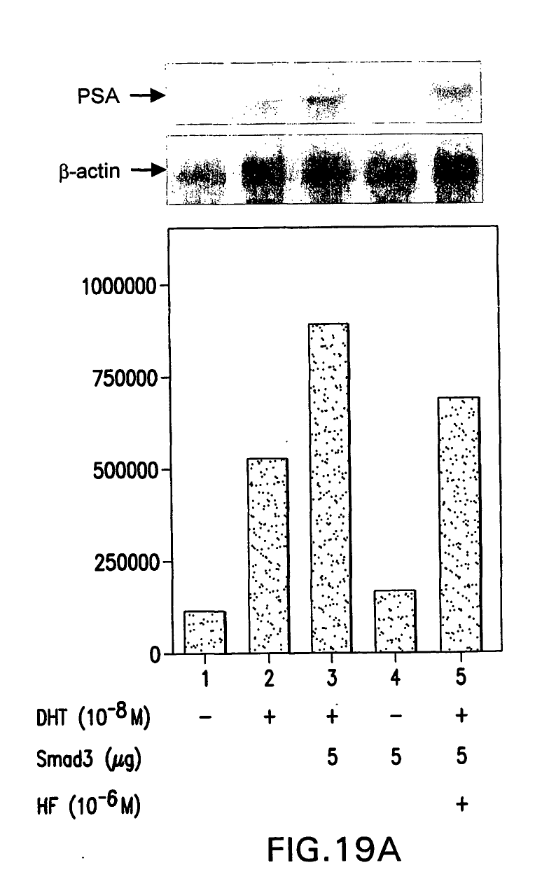

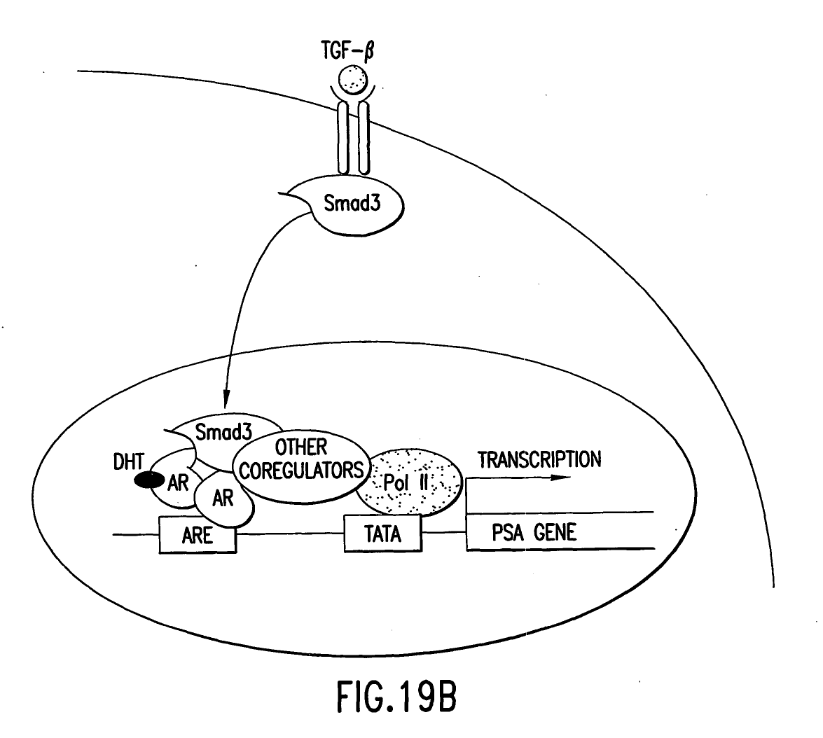

25. FIG. 19 shows AR-induced PSA expression is potentiated by Smad3. (A) Smad3 enhanced androgen/AR-induced PSA mRNA expression. LNCaP cells were transfected with Smad3 and parent vector as indicated for 16 h, followed by DHT treatment for another 16 h. PSA expression level was determined by Northern blotting. The probe was obtained from exon 1 of the PSA gene and labeled with [α-³²P] dCTP. A β-actin probe was used as a control for equivalent mRNA loading. (B) A model for androgen and TGF-β pathways in AR-mediated PSA transcription.

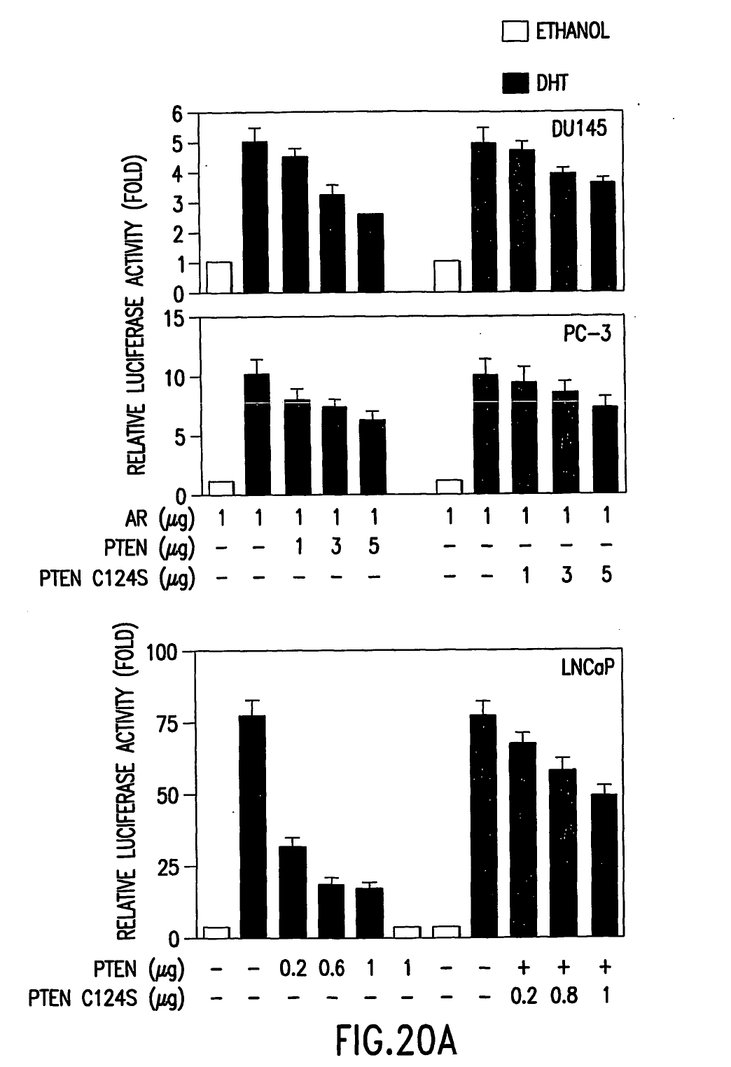

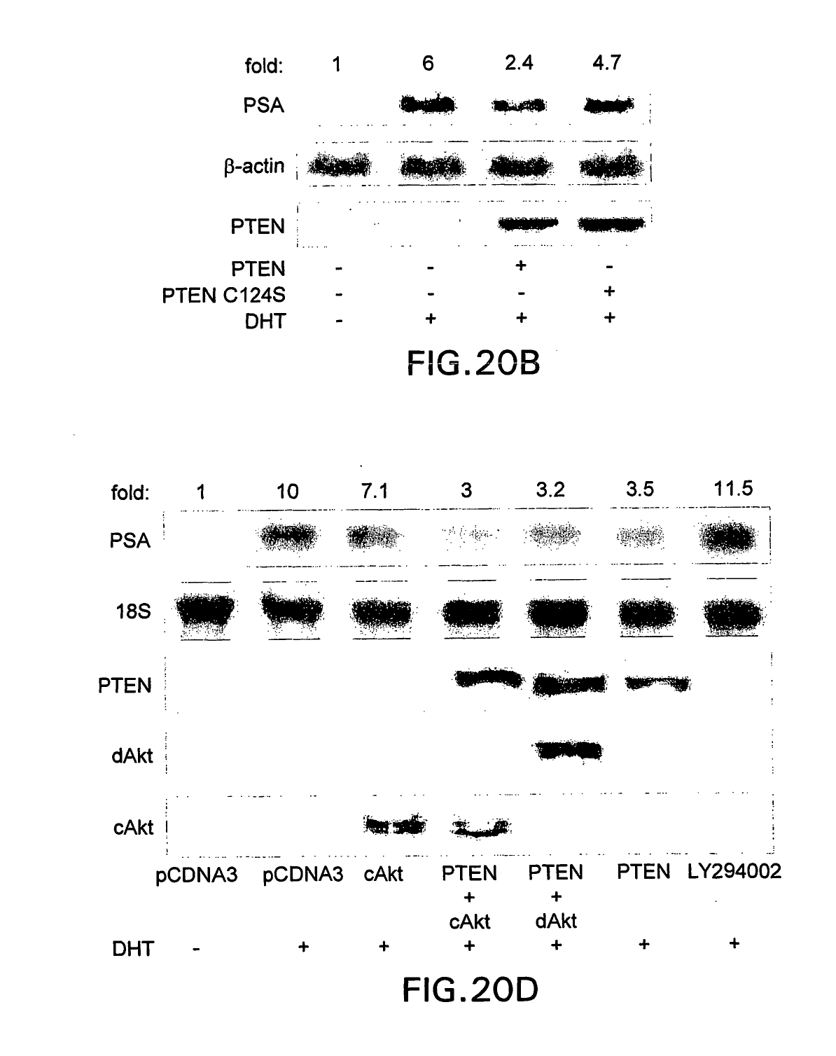

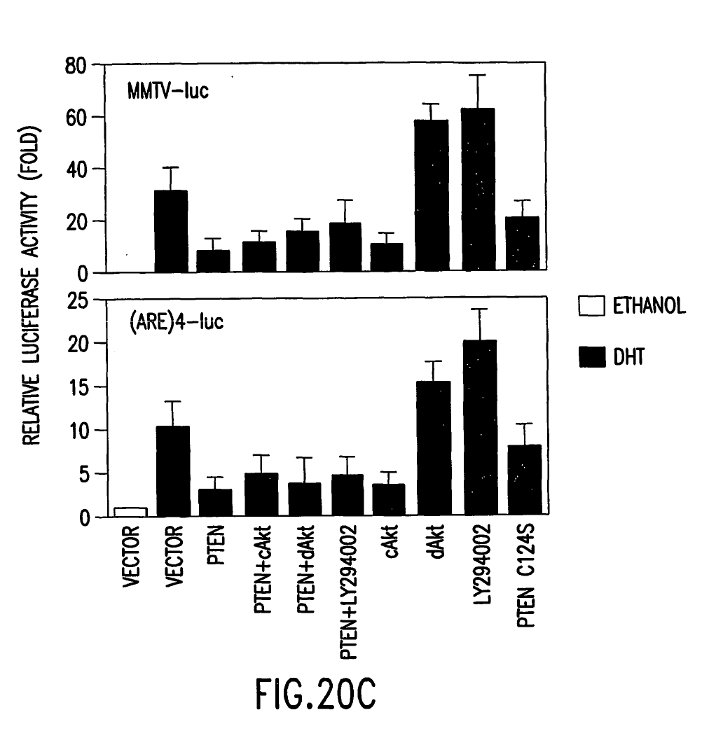

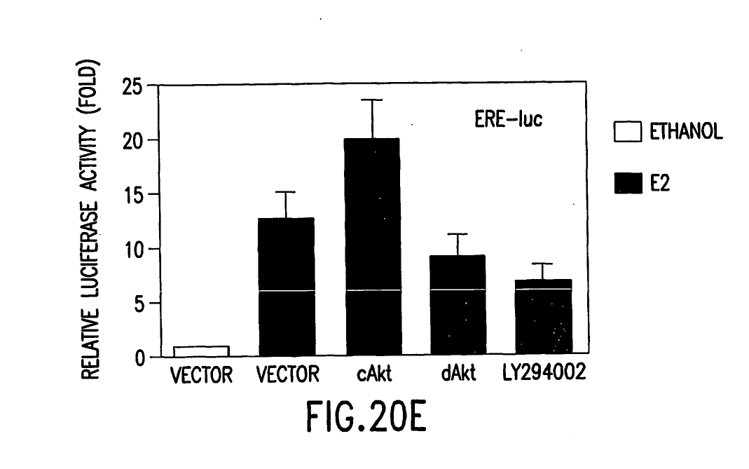

26. FIG. 20 shows PTEN suppresses AR transactivation involving the pathways other than PI3K/Akt (A) The LNCaP, PC-3, or DU145 cells were transfected with plasmids, as indicated, in 10% CDS media for 16 h and treated with 10 nM DHT for another 16 h. The cells were harvested and assayed for luciferase activity using MMTV-luc as a reporter. (B) LNCaP cells were transfected with plasmids as indicated for 24 h and then treated with DHT for another 24 h. The cells were harvested for Northern blot analysis. (C) LNCaP cells were transfected with plasmids as indicated in 10% CDS media for 16 h and then treated with 10 nM DHT for another 16 h. The cells were harvested and assayed for the luciferase activity. (D) LNCaP cells were transfected with plasmids as indicated in 10% CDS media for 24 h and then treated with DHT for 24 h The cells were harvested for Northern blot analysis. (E) The PC-3 cells were transfected with ERα along with plasmids, as indicated, using ERE-luc as a reporter for 16 h, followed by 10 nM E2 treatment for another 16 h. The cells were harvested for luciferase assay. Data are means±s.d. from three independent experiments.

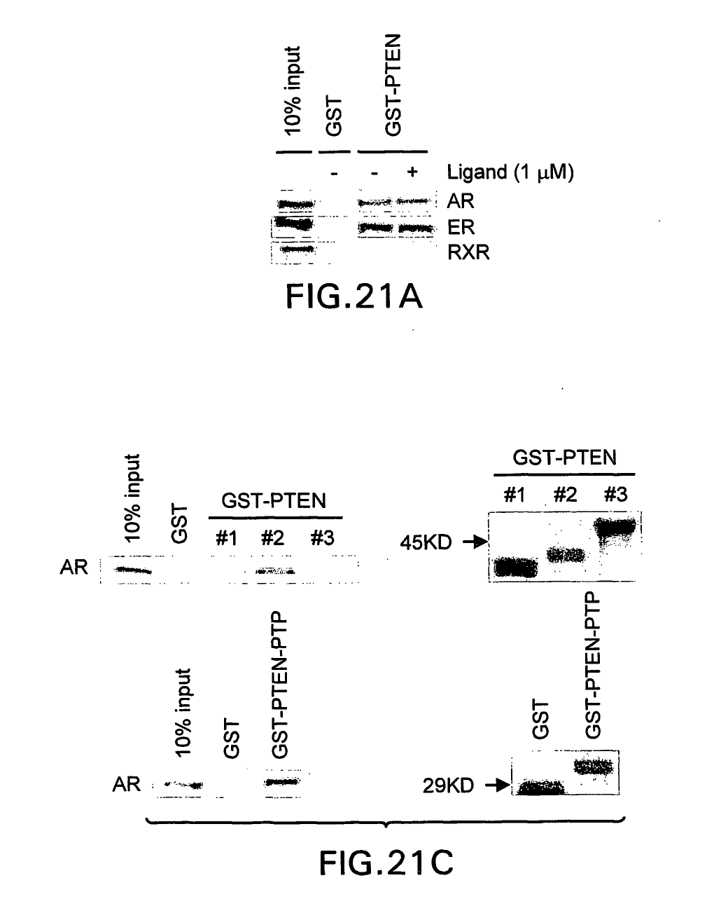

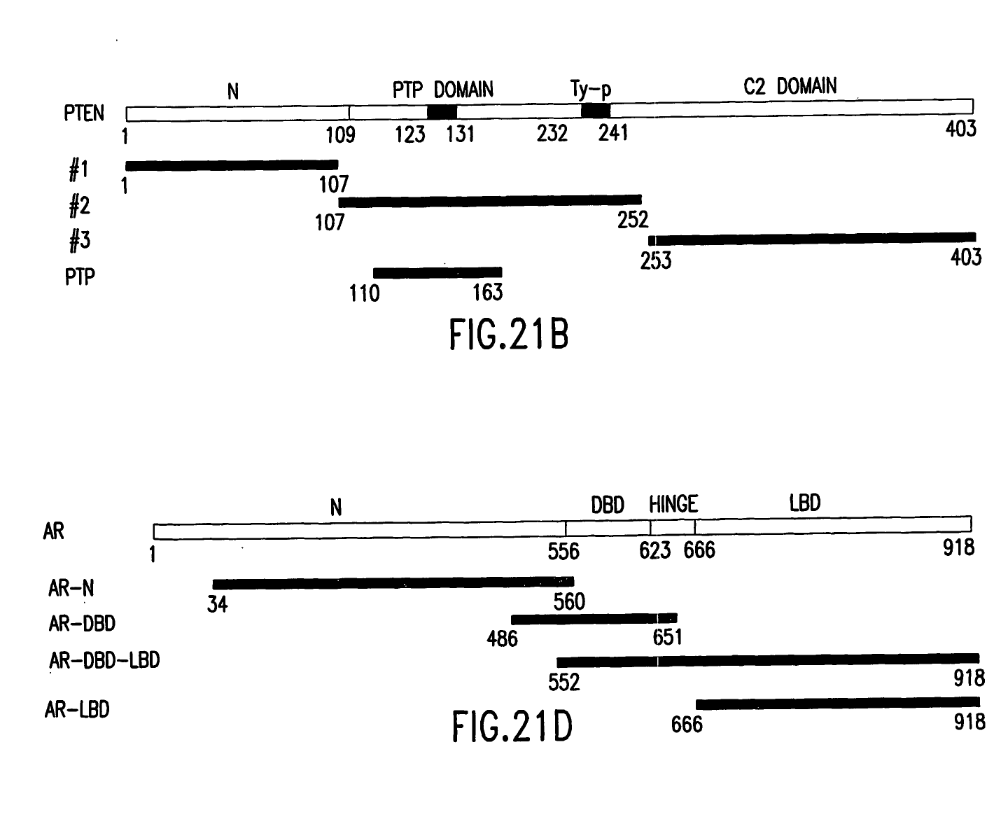

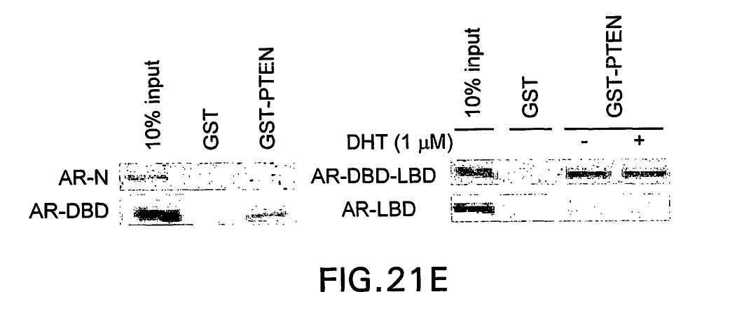

27. FIG. 21 shows PTEN interacts with AR in vitro. (A) GST or GST-PTEN incubation with the [³⁵S]-labeled AR, ER, or RXR for 2 h in the presence or absence of the ligand. The bound proteins were analyzed by SDS-PAGE, followed by autoradiography. (B) Representation of PTEN deleted mutants. PTP domain, protein tyrosine phosphates domain; Ty-p, tyrosine phosphorylation domain. (C) [³⁵S]-labeled AR was incubated with different PTEN deleted mutants. The nearly equivalent aliquots of PTEN deleted mutants used are shown in the right panel. (D) Representation of AR deleted mutants. (E) GST or GST-PTEN was incubated with different AR deleted mutants. (F) The His-AR-N-DBD was incubated with [³⁵S]-PTEN or [³⁵S]-PTEN C124S for 2 h. The bound protein was analyzed by the SDS-PAGE, followed by autoradiography.

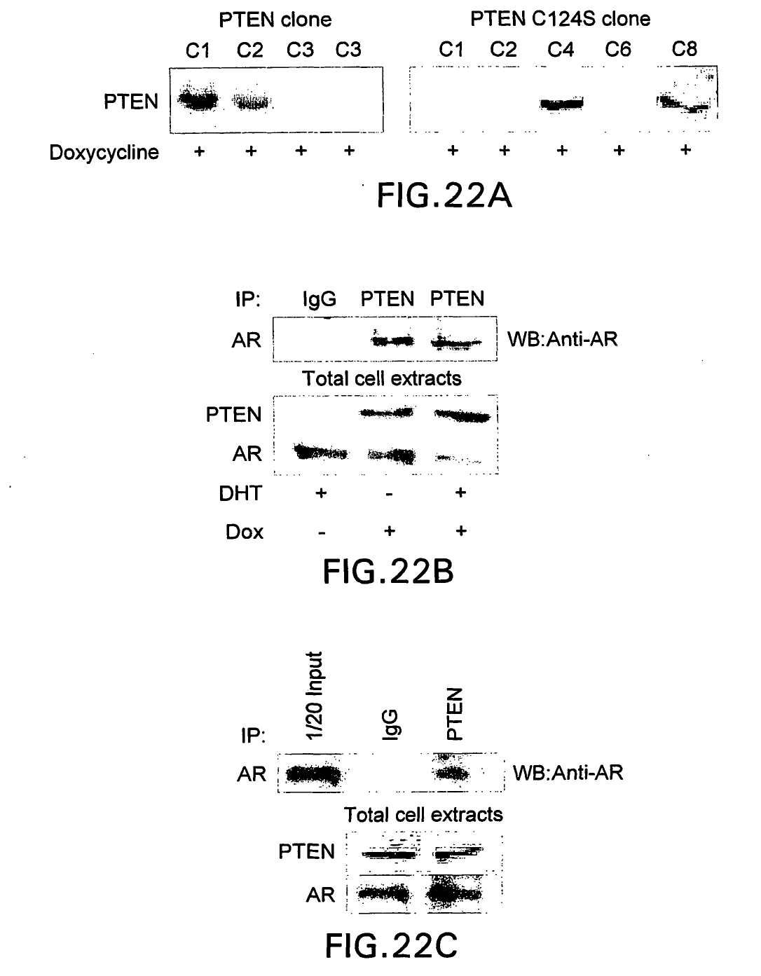

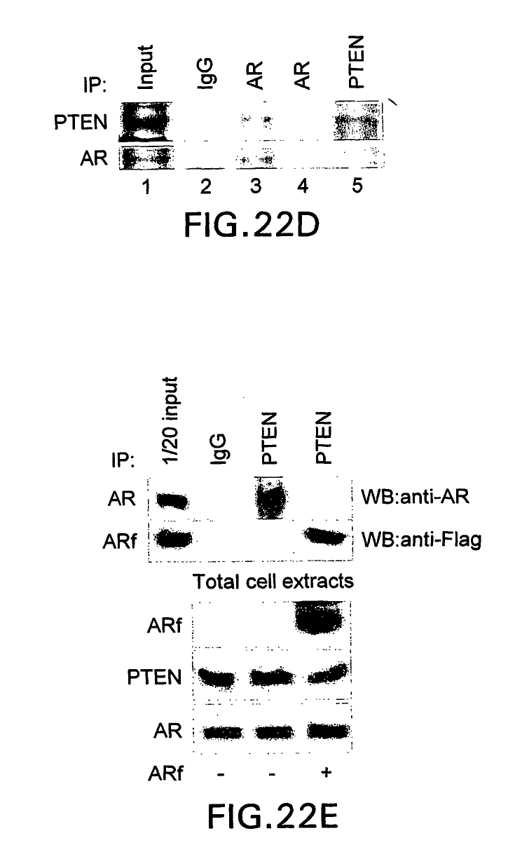

28. FIG. 22 shows PTEN interacts with AR in vivo. (A) The establishment of stable PTEN and PTEN C124S clones in LNCaP cells by Dox-inducible system The cells were treated with 4 μg/ml Dox for 24 h and harvested for western blot analysis using PTEN antibody. (B) The stable PTEN clone 1 (PTEN-C1) was treated with 4 μg/ml Dox in 10% CDS media for 24 h and treated with ethanol or DHT for another 24 h The cells were harvested for immunoprecipitation assay. (C) The cell lysates from the CWR22 were immunoprecipitated by anti-PTEN antibody, followed by Western blot analysis. (D) The CWR22 cells were labeled by [³⁵S]-methionine for 4 h in the presence of the 10 nM DHT. The cells were harvested for co-immunoprecipitation. Lane 1: 10% input; Lane 2: IP by non-immune IgG; Lane 3: IP by anti-AR antibody; Lane 4: The PTEN protein was depleted from the cell lysates by anti-PTEN antibody before IP by anti-AR antibody; Lane 5: IP by anti-PTEN antibody. (E) CWR22 cells were transfected with vector or Akf for 48 h and the cells were harvested for immunoprecipitation assay.

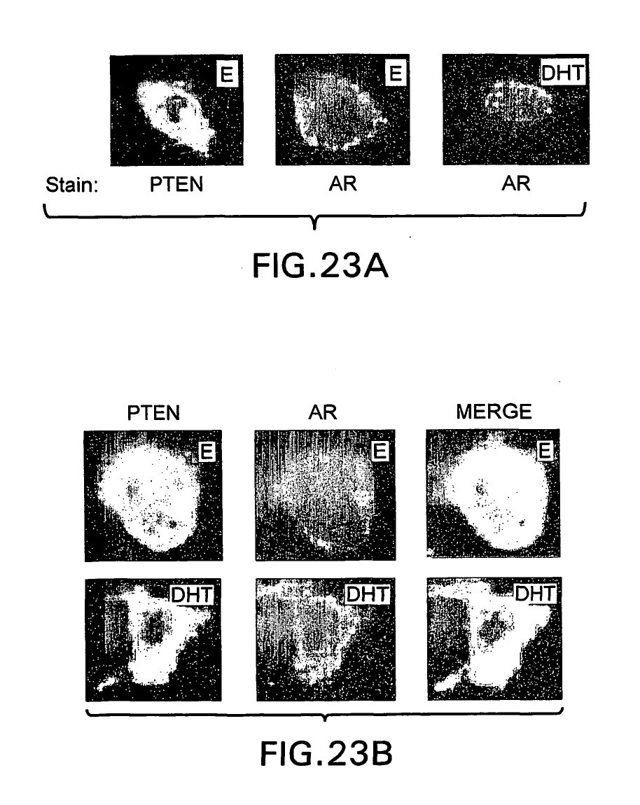

29. FIG. 23 shows PTEN colocalizes with AR in vivo. (A) The COS-1 cells were transfected with AR or PTEN in 10% CDS media for 16 h and treated with ethanol or 10 nM DHT for another 16 h. The cells were fixed and stained with AR and PTEN antibodies, followed by examination with confocal microscopy. (B) The COS-1 cells were transfected with AR and PTEN and treated with ethanol or 10 nM DHT for another 16 h. The cells were fixed and stained with AR and PTEN antibodies, followed by examination with confocal microscopy. The green and red colors represent PTEN and AR staining, respectively, and the yellow color represents PTEN and AR colocalization.

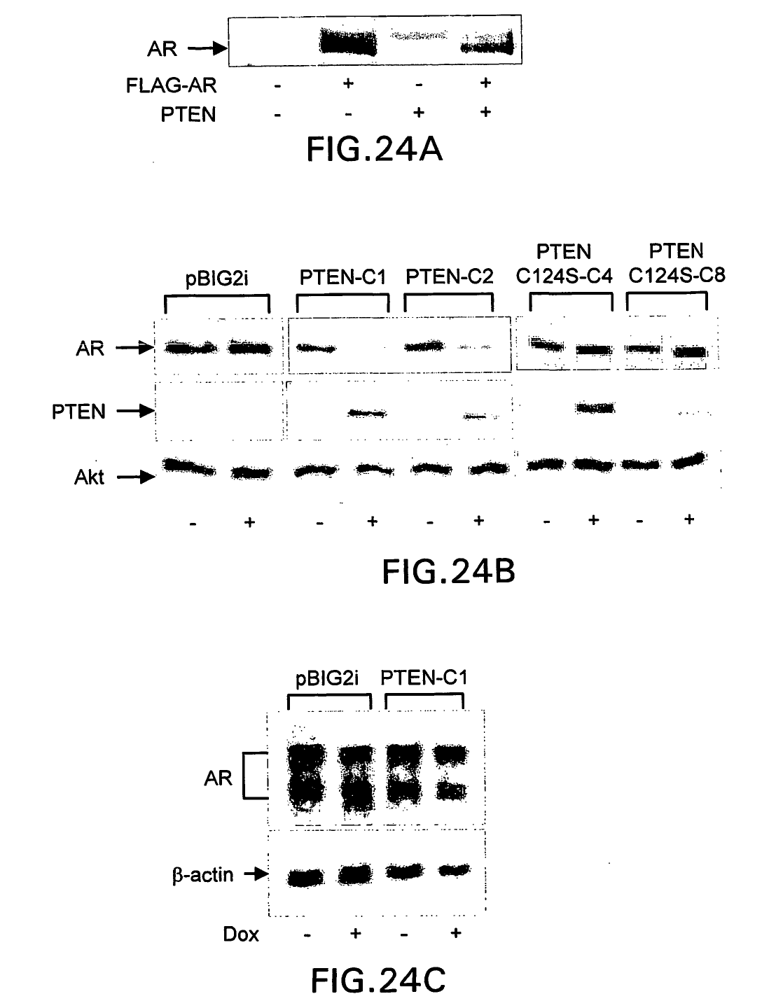

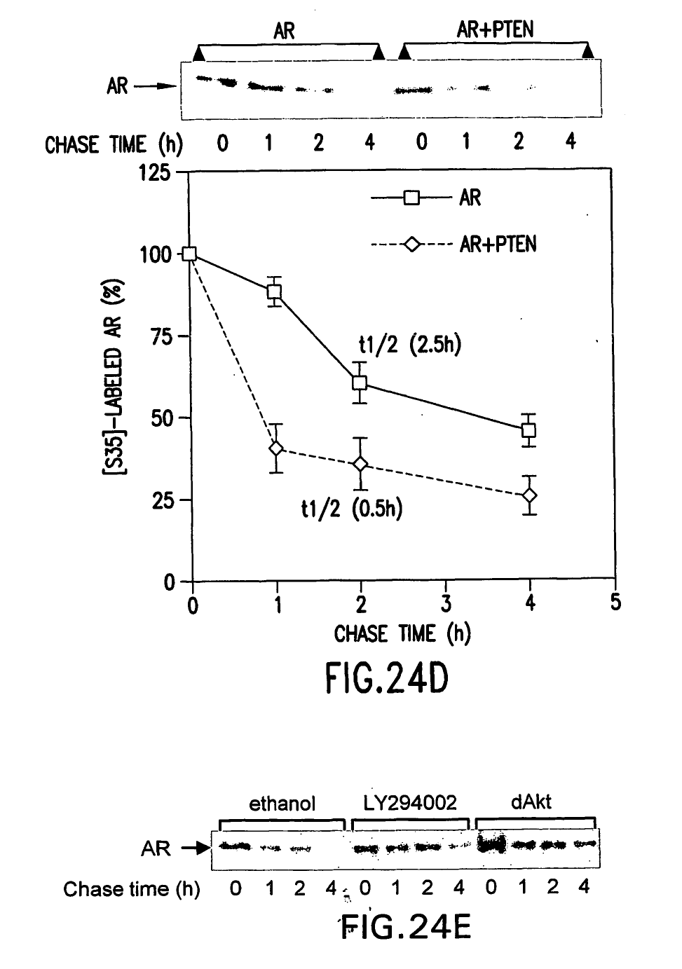

30. FIG. 24 shows PTEN decreases AR protein level via promotion of AR degradation (A) COS-1 cells were transfected with AR with a flag epitope in front of the AR sequence, along with pCDNA3 or PTEN in 10% CDS media for 16 h. The cells were harvested for Western blot analysis. (B) LNCaP cells stably transfected with vector, PTEN, or PTEN-C124S were treated with 4 μg/ml doxycycline in 10% CDS media for 48 h in the presence of 10 nM DHT. Western blot analysis was performed and AR and PTEN were detected by AR antibody or PTEN antibody. (C) PTEN-C1 cells were treated with 4 μg/ml Dox in 10% CDS media for 48 h. Total RNA was prepared and Northern blot analysis was performed using [³²P]-dCTP-labeled AR (aa, 1-389) as a probe. (D) COS-1 cells were transfected with AR along with pCDNA3 or PTEN in 10% CDS media for 16 h. The cells were then pulsed with [³⁵S]-methionine for 45 min in the presence of 10 nM DHT and harvested at different chase times as indicated. The cell extracts were immunoprecipitated with AR antibody and subjected to SDS-PAGE followed by autoradiography. Data were from three identical results. (E) COS-1 cells were transfected with AR along with pCDNA3 or dAkt in 10% CDS media for 16 h, pulsed with [³⁵S]-methionine for 45 min, and then harvested at different chase times as indicated. LY294002 (20 μM) was added 2 h before pulsing with [³⁵S]-methionine.

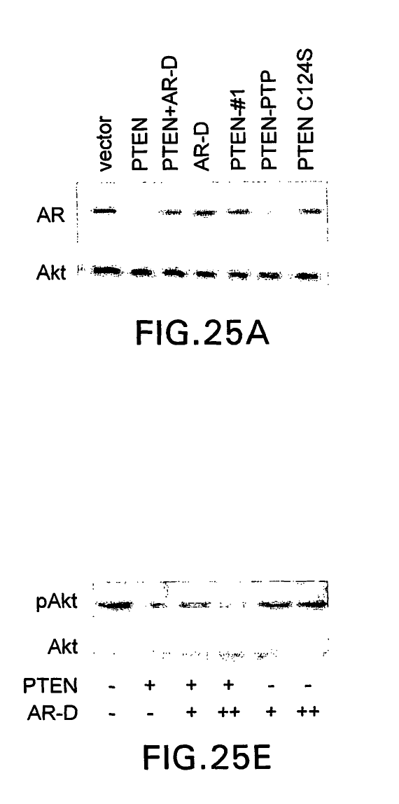

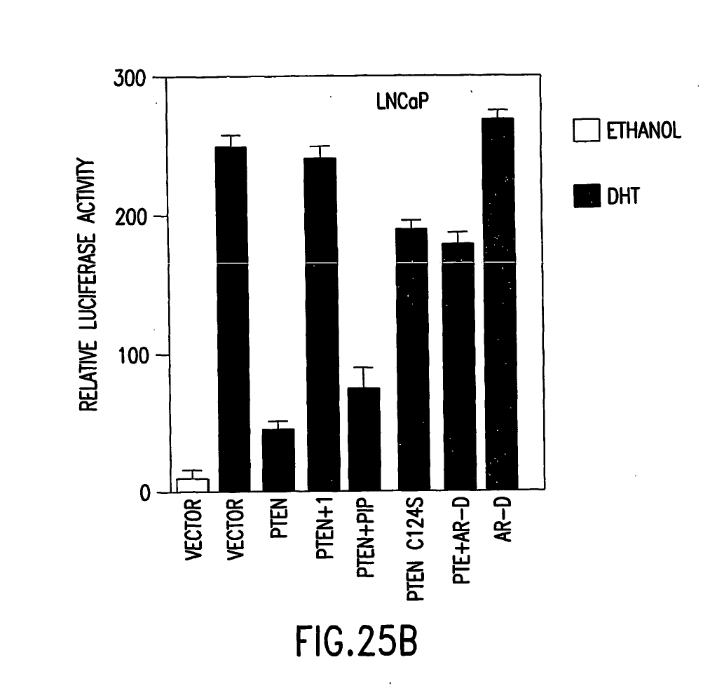

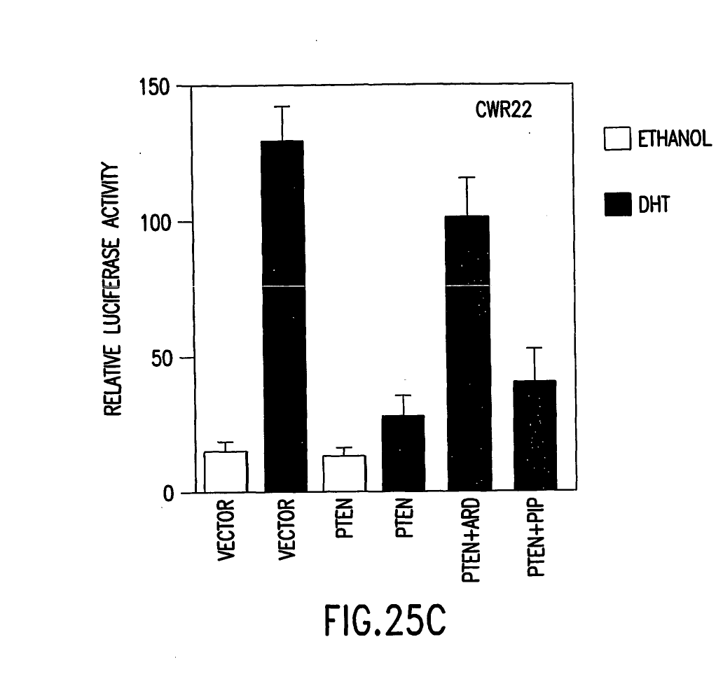

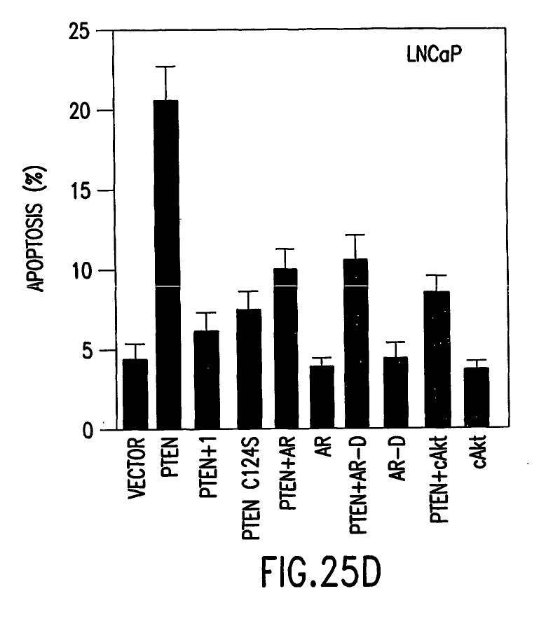

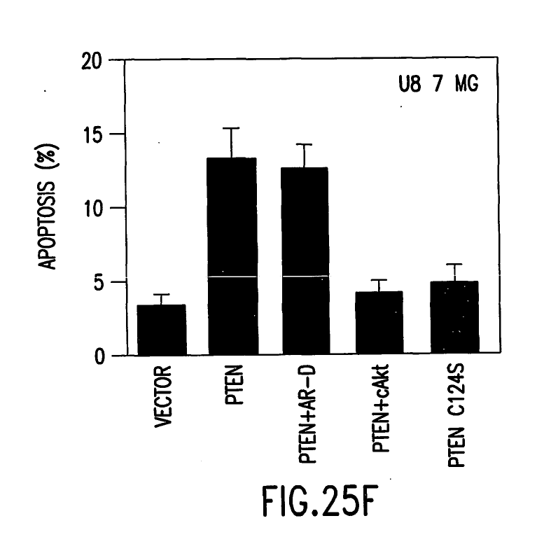

31. FIG. 25 shows the interaction between PTEN and AR contributes to PTEN-induced suppression of AR functions and apoptosis. (A) The LNCaP cells were transfected with plasmids in 10% CDS media, as indicated for 16 h, treated with 10 nM DHT for 24 h, harvested and the cell extracts were subjected to SDS-PAGE. (B) The LNCaP cells were transfected with plasmids in 10% CDS media, as indicated for 16 h and then treated with 10 nM DHT for another 16 h, harvested and assayed for MMTV-luciferase activity. (C) The CWR22 cells were transfected with plasmids as indicated using (ARE)4-luc as a reporter for 16 h, followed by ethanol or 10 nM DHT treatment for another 16 h, and harvested for luciferase assay. (D) The LNCaP cells were transfected with plasmids as indicated for 16 h, and the medium was changed to 0.1% CDS media for 2 days. The cell apoptosis was determined by TUNEL assay (E) The LNCaP cells were transfected with plasmids as indicated for 16 h, harvested, and the cell extracts were subjected to SDS-PAGE. The Akt activity was determined by Western blotting using phospho(S473)-Akt antibody. (F) The U87MG cells were transfected with plasmids as indicated for 16 h, and the medium was changed 0.1% CDS media for 2 days. The cell apoptosis was determined by TUNEL assay. Data for luciferase activity and apoptosis are means±s.d. from three independent experiments.

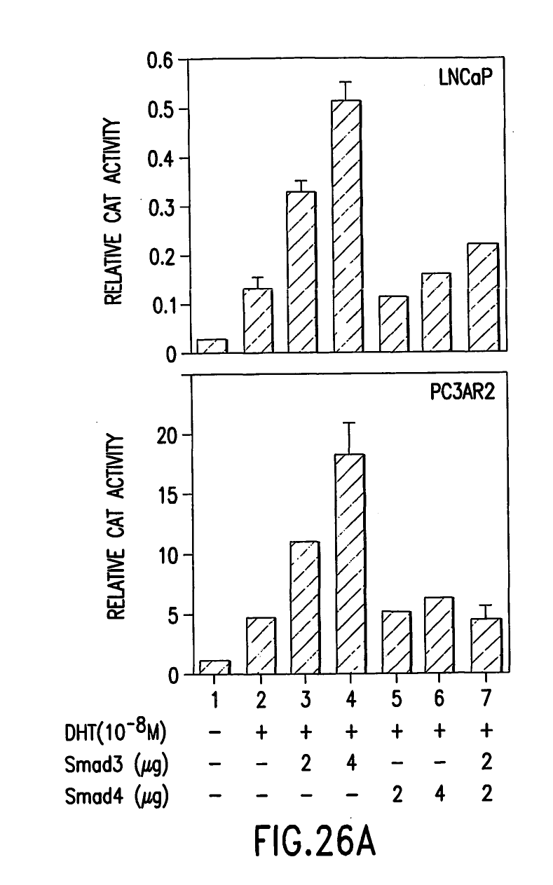

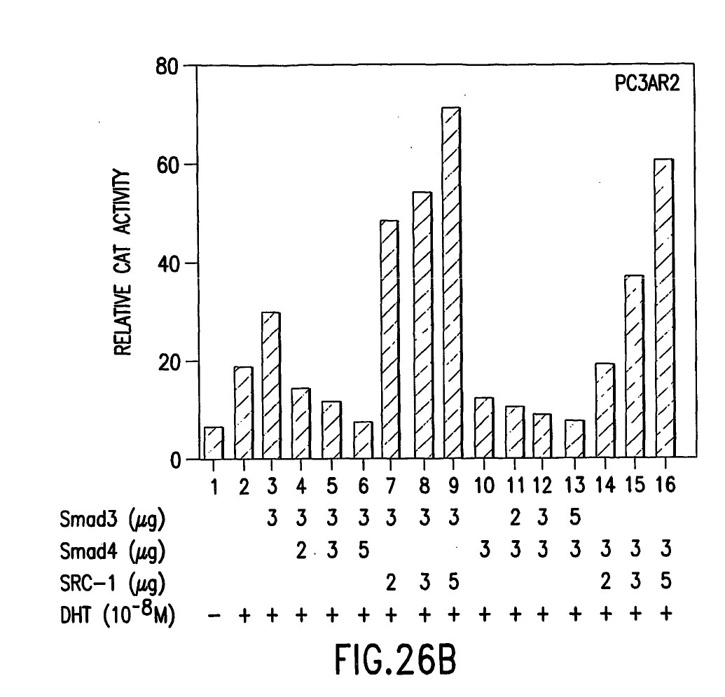

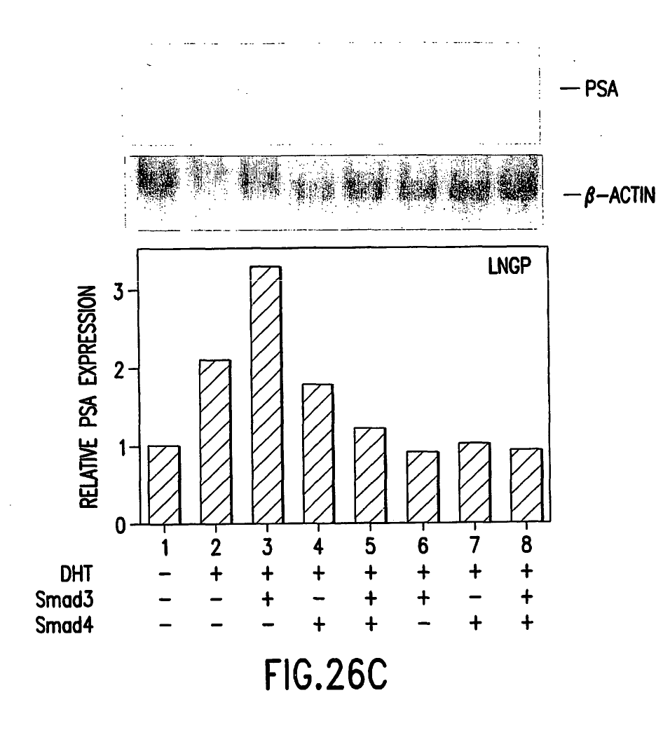

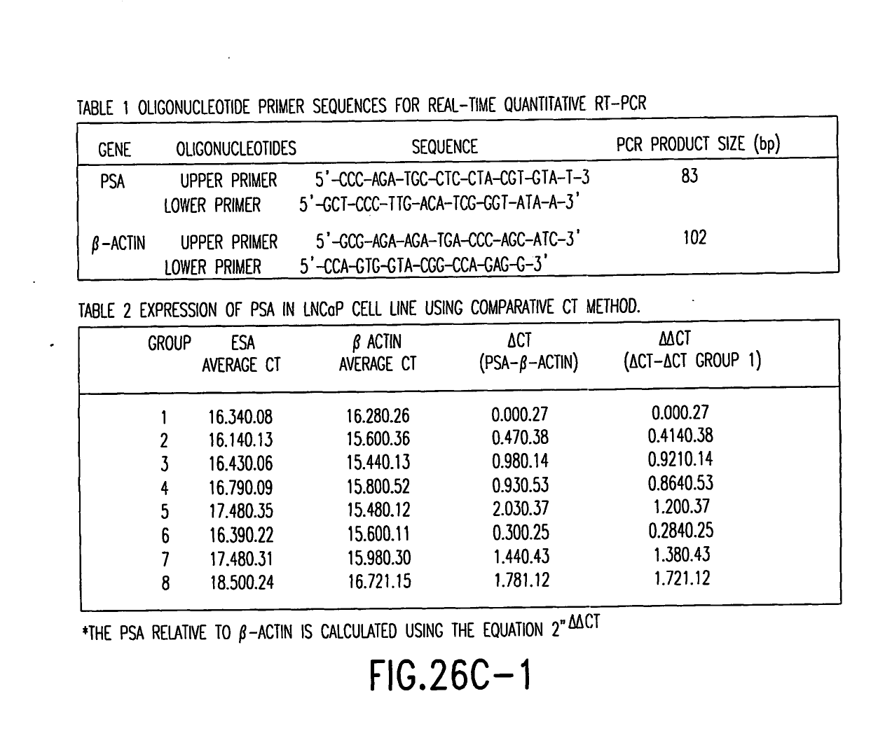

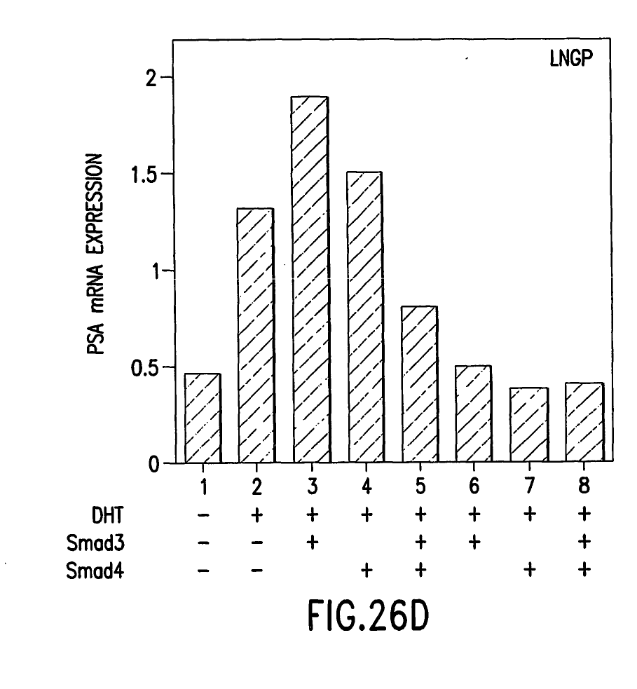

32. FIG. 26 shows ligand-induced transactivation of AR is enhanced by Smad3 but repressed by Smad3/Smad4. (A) CAT assays were performed with extracts from PC-3-AR)2 cells transfected with the indicated amount of Smad3 or Smad4 expression vector (μg) in the presence (+) or absence (−) of 10⁻⁸ M DHT. LNCaP cell were transfected with Smad3 or Smad4 expression vector instead of PC-3(AR)2 cells in experiments otherwise identical with those in A (B), PC-3(AR)2 cells stably transfected with AR were over-expressed with the indicated amounts of Smad3 or Smad4 or SRC-1. 3 μg of MMTV-CAT was used as a reporter plasmid in all experiments. All values represent the averages±SD of four independent experiments. (C) LNCaP cells were transfected with Smad3, Smad4, and parent vector as indicated for 16 h, followed by DHT treatment for another 16 h. PSA expression level was determined by Northern blotting. The probe was obtained from exon 1 of the PSA gene and labeled with [α-³²P] dCTP. A β-actin probe was used as a control for equivalent mRNA loading. (C, Table 1) The RNA samples from (A) were reverse transcribed to cDNAs and all of the samples were subjected to real-time CR using an ABI Prism 7700 Sequence Detection System (Perkin-Elmer Applied bio systems). PCR was performed using the SYBR Green PCR Core Reagents kit (Perkin-Elmer Applied Bio systems). PSA and β-actin forward, reverse primers concentrations were 2.5 μM. To reduce variability between replicates, PCR premixes which contained all reagents except for total RNA were prepared and aliquoted into 1.5 ml microfuge tubes. Specific PCR amplification products were detected by the fluorescent double-stranded DNA-binding dye SYBR Green core reagent kit. Experiments were performed with triplicates for each data point.

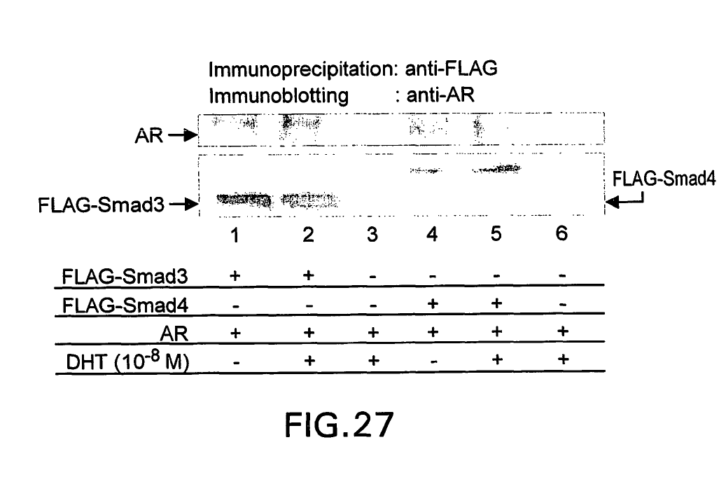

33. FIG. 27 shows an In vivo interaction between Smads and AR. (A) Co-immunoprecipitation of AR and Smad3. (B) PC-3(AR)2 cells that overexpress Flag-Smad3, Flag-Smad4 and AR were treated with and without DHT. Of 10 nM DHT to test for interaction domains of AR for Smad3 and Smad4. Complex bound to GST columns was subjected to SDS-PAGE and autoradiography. (B) GST-AR-LBD fusion proteins and GST control were incubated with in vitro transcribed/translated [³⁵S]-labeled Smad3 or Smad4 in the presence (+) or absence (−) of 10 nM DHT to test for interaction in the GST pull-down assay. (C) The indicated amounts of purified Smad4 proteins were added to fixed volume of in vitro transcribed/translated [³⁵S]-labeled full-length AR and 2 μg GST-Smad3 to test for interaction. Bands were detected using a phosphorimager. A representative blot of three independent experiments is shown.

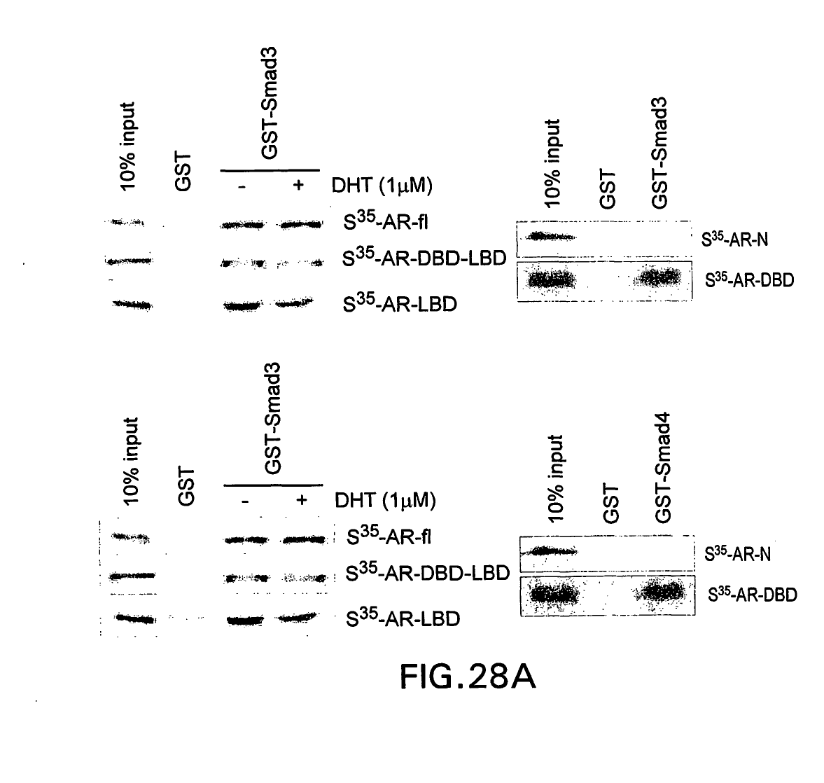

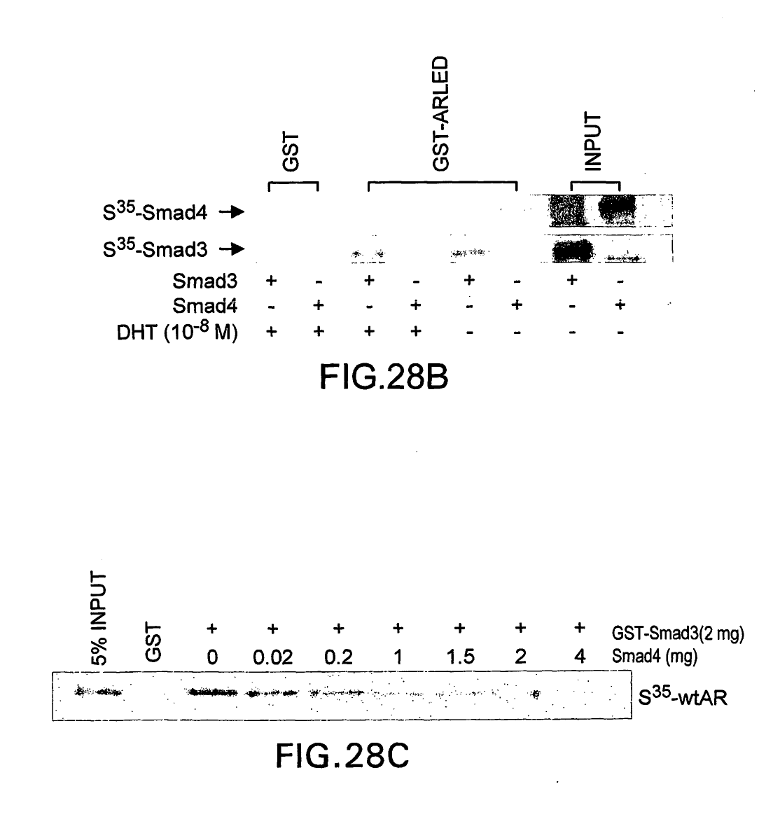

34. FIG. 28 shows the in vitro interaction between Smads and AR. (A) A series of [³⁵S]-labeled full-length AR and different AR deletion mutatnts were incubated with GST-Smad3, GST-Smad4 and GST alone in the presence (+) or absence (−) of 10 nM DHT to test for interaction domains of AR for Smad3 and Smad4. Complex bound to GST columns was subjected to SDS-PAGE and autoradiography. (B) GST-AR-LBD fusion proteins and GST control were incubated with in vitro transcribed/translated [³⁵S]-labeled Smad3 or Smad4 in the presence (+) or absence (−) of 10 nM DHT to test for interaction in the GST pull-down assay. (C) The indicated amounts of purified Smad4 proteins were added to fixed volumes of in vitro transcribed/translated [³⁵S]-labeled full-length AR and GST-Smad3 (2 μg) to test for interaction. Bands were detected using a phosphoimager. A representative blot of three independent experiments is shown.

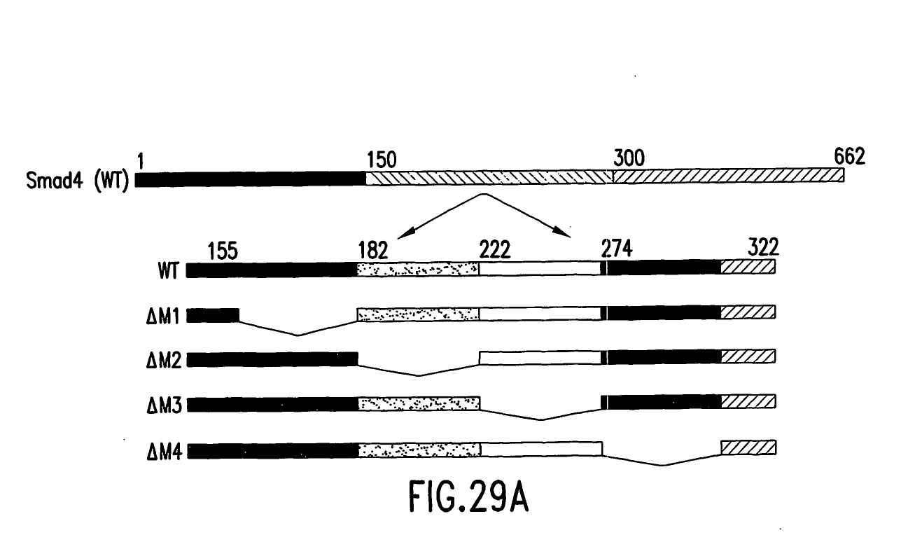

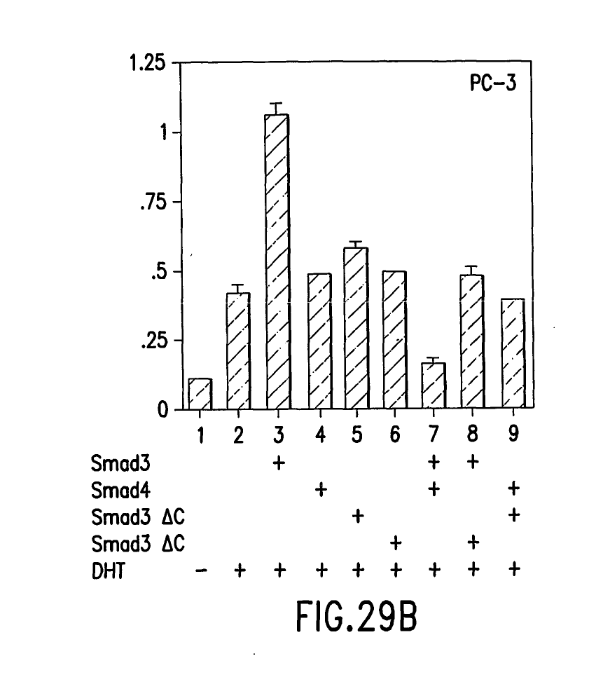

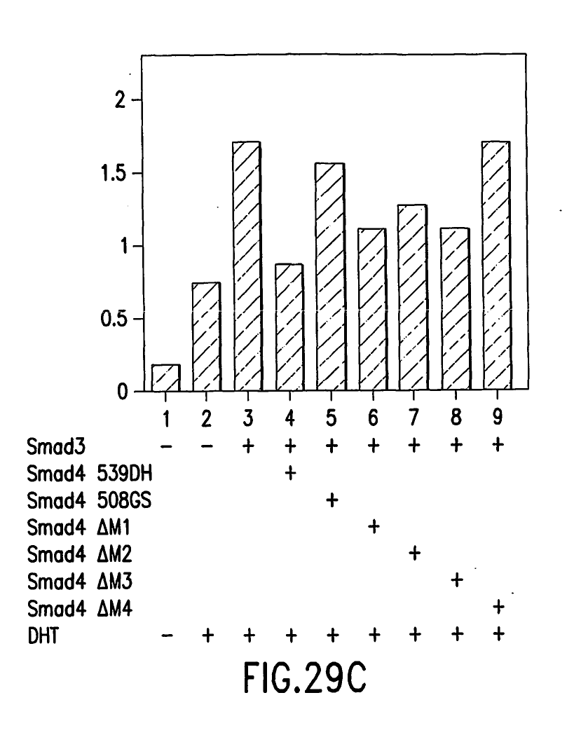

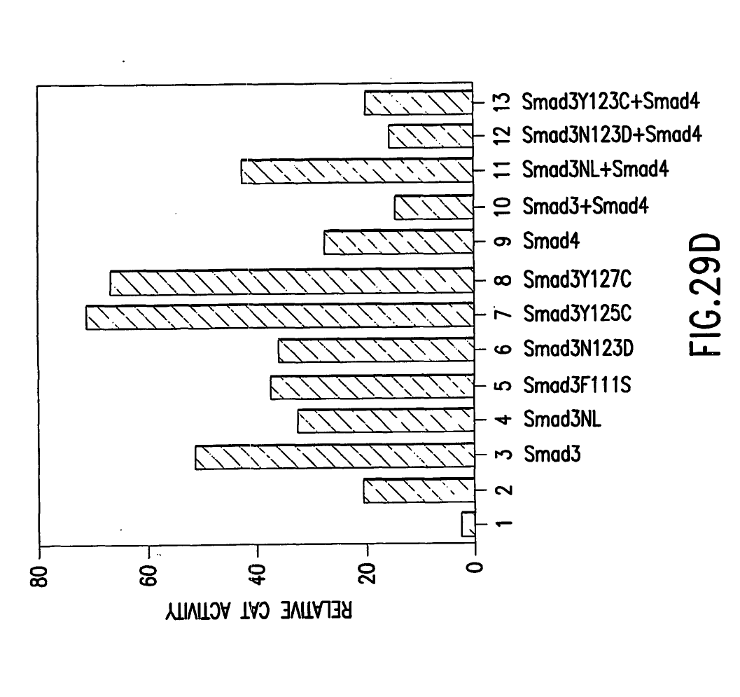

35. FIG. 29 shows an association of Smad3 and Smad4 with AR deacylation. (A) PC3-AR2 cells co-transfected with the MMTV-LUC reporter and the expression vector for the Smad3/Smad4 were treated with DHT in the presence of increasing doses of TSA. All values represent the mean±SD of three independent experiments. (B) Immunoprecipitation assays were performed with extracts from PC-3(AR)2 cells transfected with Smad3, Smad4, or Smad3/Smad4 expression vector in the presence (+) or absence (−) of 10⁻⁸ M DHT. Equal amounts of whole-cell lysates were subjected to co-immunoprecipitation with an anti-AR antibody. The co-immunoprecipitates were analyzed on Western blot with a specific anti-acetyl lysine antibody. Anti-AR or anti-flag was used in immunoblot analysis of the immunoprecipitated complexes to confirm that uniform amounts of AR and Smads were immunoprecipitated.

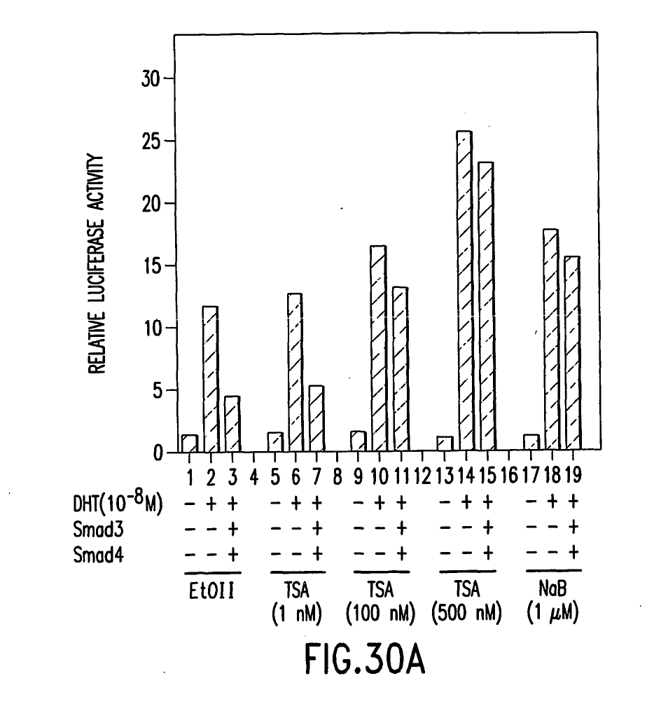

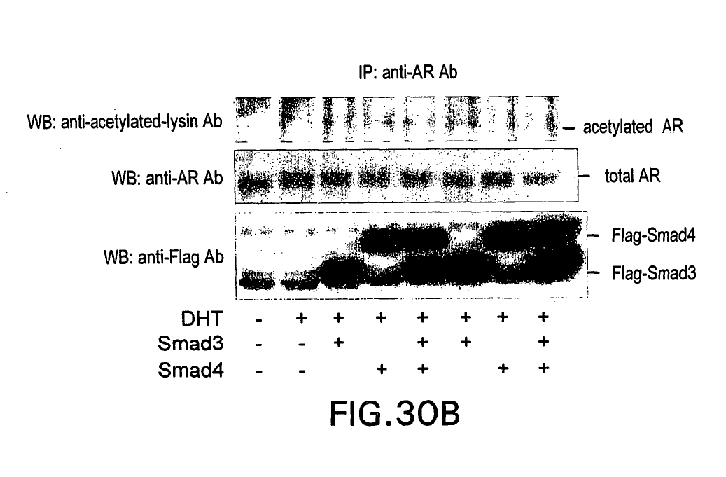

36. FIG. 30 shows effects of Smad3 and Smad4 mutants on AR-mediated transcriptional activity. (A) The wtSmad4 and different Smad4 deletion mutants used are shown schematically. (B) PC-3 cells were co-transfected with 3 μg of Smad3, Smad4, Smad3ΔC or Smad4ΔC mutant expression vectors with 1 μg of pSG5-AR and 3 μg of MMTV-CAT, in the presence (+) or absence (−) of 10⁻⁸ M DHT. (C) Smad3 was co-expressed with different Smad4 deletion mutants as indicated in PC-3 cells. Each CAT activity is presented relative to the transactivation observed in the absence of DHT and an error bar represents the mean±SD of four independent experiments.

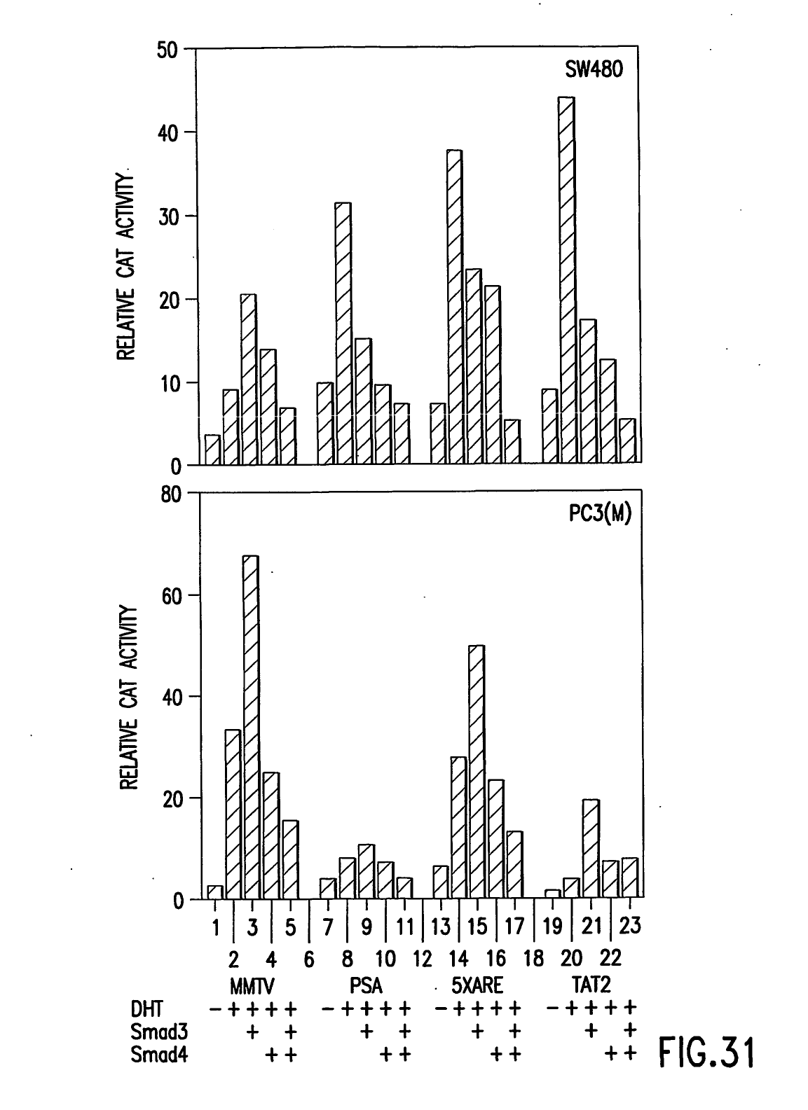

37. FIG. 31 shows that an androgen-response element is important for TGF-β/Smad3-enhanced AR transactivation. (A) PC3-AR2, SW480·C7, and PC-3 cells were transiently co-transfected with either MMTV-CAT, PSA-CAT, 5XARE-CAT, or (TAT)2-CAT (3 μg), in the presence of AR, Smad3, Smad4 or Smad3/Smad4 as indicated. Each CAT activity is presented relative to the transactivation observed in the absence of Smad3. All values represent the mean±SD of three independent experiments.

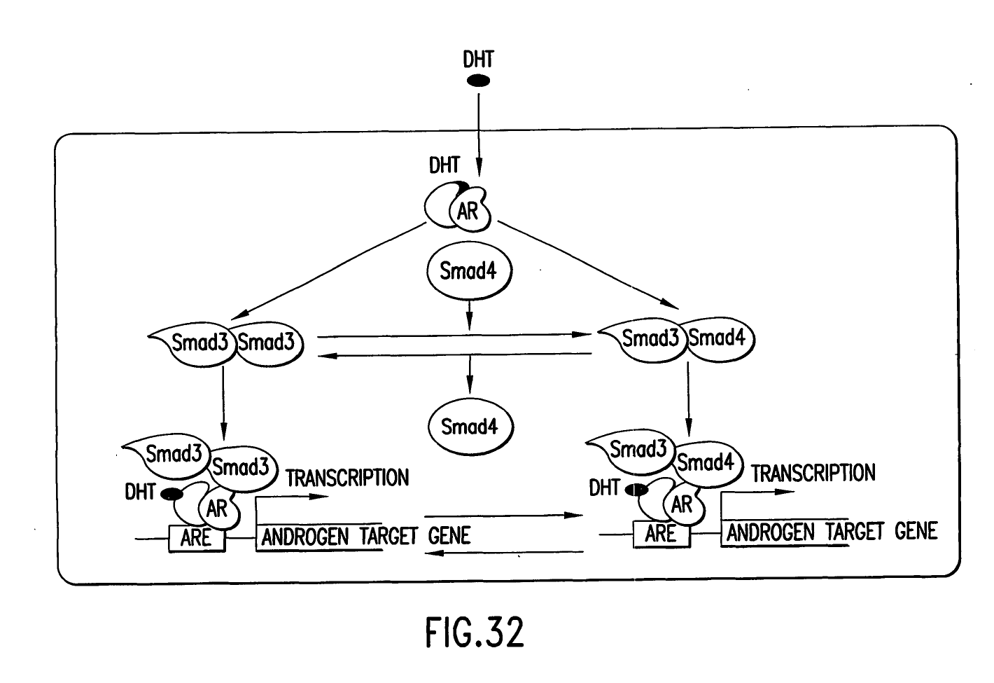

38. FIG. 32 shows a model for the roles of Smad3 and Smad4 in AR-mediated target genes transactivation.

### IV. DETAILED DESCRIPTION

39. The present invention may be understood more readily by reference to the following detailed description of preferred embodiments of the invention and the Examples included therein and to the Figures and their previous and following description.

40. Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

41. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

42. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is understood that any use of a value is also considered to be disclosed as “about” the particular value. For example, if the value is “10,” also disclosed is “about 10.”

43. It also understood that a number of values are disclosed. It is understood that for all values or “at least,” “greater than or equal to,” “greater than,” “less than or equal to,” and “less than, ” the value are also disclosed. For example if the value “10” is disclosed, than at least 10,” “greater than or equal to 10,” “greater than 10,” “less than or equal to 10,” and “less than 10,” are also specifically disclosed.

44. In this specification and in the claims which follow, reference will be made to a number of terms which shall be defined to have the following meanings:

45. “Optional” or “optionally” means that the subsequently described event or circumstance may or may not occur, and that the description includes instances where said event or circumstance occurs and instances where it does not.

46. The abbreviations used are: AR, androgen receptor; TGF-β, transforming growth factor β; MH2, Mad homology 2; wtAR, wild-type androgen receptor; mtAR, mutant AR; ARA, androgen receptor associated protein; ARE, androgen response element; DHT, 5α-dihydrotestosterone; HF, hydroxyflutamide; DBD, DNA-binding domain; LBD, ligand-binding domain; PSA, prostate specific antigen; MMTV, mouse mammary tumor virus; CAT, chloramphenicol acetyltransferase; GST, glutathione sepharose transferase; CT, threshold cycle; TSA, trichostatin A.

47. Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon. Various references are listed by number in the specification. These references are also typically associated with a letter. That letter refers to the letter designation of a particular reference list included with this specification. For example, a designation “(1B)” would refer to reference 1 in list B. If the number designation is not associated with a particular number it will be clear to one of skill which reference is being referred to by the context the reference is relied upon and by a review of the various possible references. The examples all refer to a particular set of references, and thus do not have a letter designation associated with individual numbers.

48. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

49. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular AR is disclosed and discussed and a number of modifications that can be made to a number of molecules including the AR are discussed, specifically contemplated is each and every combination and permutation of AR and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-B, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

D. Compositions and Methods

50. Disclosed are methods for modulating androgen receptor-mediated transactivation activity in a cell, comprising the step of: treating the cell with an agent that modulates the activity of a pathway from or to androgen receptor-androgen receptor coactivator interaction.

51. Modulating means changing relative to a control. The control is based on what is being tested. For example, if one is modulating adrogen receptor transcription activity, then a control would be a standard androgen receptor transcription activity assay as described herein. As another example, if one were modulating PSA levels, then a control would be any standard PSA assay as described herein. Thus, some activity or some level, for example, are modulated if any change occurs in the activity or in the level relative to a control as described herein.

52. It is also understood that there are levels of modulation that can occur when measuring any assay. For example, modulation can be at least a 1.5 fold, at least a 2 fold, at least a 2.5 fold, at least a 3 fold, at least a 3.5 fold, at least a 4 fold, at least a 4.5 fold, at least a 5 fold, at least a 6 fold, at least a 7 fold, at least a 8 fold, at least a 9 fold, at least at least a 10 fold, at least a 15 fold, at least a fold, at least a 25 fold, at least a 30 fold, at least a 50 fold, at least a 100 fold, at least a 500 fold, at least a 1000 fold difference between the control and the activity or substance, for example, being tested.

53. Also disclosed are methods, wherein the agent modulates phosphatidylinositol 3-kinase activity, wherein the agent modulates Akt/PKB activity, wherein the agent is LY249002, wherein the agent is a constitutively active form of phosphatidylinositol 3-kinase, wherein the constitutively active form of phosphatidylinositol 3-kinase is p110, wherein the agent is a dominant negative form of phosphatidylinositol 3-kinase, wherein the dominant negative form of phosphatidylinositol 3-kinase is Δp85, wherein the agent is a constitutively active form of Akt/PKB, wherein the constitutively active form of Akt/PKB is cAkt, wherein the agent is a dominant negative form of Akt/PKB, and/or wherein the dominant negative form of Akt/PKB is dAkt.

54. Also disclosed are methods for repressing androgen receptor-mediated transactivation activity in a cell, comprising the step of: treating the cell with IL-6 and a mitogen-activated protein kinase pathway inhibitor. It is understood that repressing is a fomr of modulation.

55. Also disclosed are methods, wherein the mitogen-activated protein kinase pathway inhibitor is PD98059.

56. Disclosed are methods for increasing androgen receptor-mediated transactivation activity in a cell, comprising the step of: treating the cell with an upstream androgen receptor activity pathway inhibitor. It is understood that increasing is a form of modulation.

57. Also disclosed are methods, wherein the phosphatidylinositol 3-kinase pathway inhibitor is LY249002, wherein the phosphatidylinositol 3-kinase pathway inhibitor is a Akt/PKB inhibitor, and/or wherein the Akt/PKB inhibitor is dAkt.

58. Disclosed are methods for screening a compound for use in treatment of androgen receptor-related diseases, comprising the step of determining the effect of the compound on one of the steps of the phosphatidylinositol 3-kinase pathway from and including phosphatidylinositol 3-kinase to and including androgen receptor-androgen receptor coactivator interaction. For example, the effect is a form of modulation.

59. The step of determining can be performed using any assay for modulation, for example, such as an assay for determining androgen recpetor transcription activity, androgen receptor ligand binding, PSA levels, or tumor growth, for example.

60. Also disclosed are methods, wherein the step of the phosphatidylinositol 3-kinase pathway is the phosphatidylinositol 3-kinase activation, wherein the step of the phosphatidylinositol 3-kinase pathway is the Akt/PKB activation, and/or wherein the step of the phosphatidylnositol 3-kinase pathway is androgen receptor serine 210 phosphorylation.

61. Disclosed are methods for modulating androgen receptor-mediated transactivation activity in a cell, comprising the step of: treating the cell with an agent that modulates the activity of a signal transduction pathway involving androgen receptor.

62. Also disclosed are methods, wherein the pathway is a Smad3 pathway, wherein the pathway is a Smad4 pathway, wherein the pathway is a Akt pathway, wherein the pathway is a PTEN pathway, wherein the pathway is a TGF-B pathway.

63. Disclosed are methods for modulating androgen receptor-mediated transactivation activity in a cell, comprising the step of: treating the cell with an agent that inhibits the interaction between androgen receptor and a protein that modulates AR activation.

64. Also disclosed are methods, wherein the protein is selected from the group consisting of Smad3, Smad4, Akt, PTEN, or TGF-B.

65. Disclosed are methods of modulating AR activity comprising an administering an that agent binds directly to AR and inhibits AR activity.

66. Also disclosed are methods, wherein the agent decreases AR activity and/or wherein the agent is PTEN.

67. Disclosed are methods of identifying an agent that modulates AR activity through direct interaction comprising assaying the interaction through a glutothionine-S-transferase pull-down assay, mapping the domains of interaction GST pull-down assays using mutated forms of the agent or mapping the domains of interaction GST pull-down assays using mutated forms of AR, Smad3, Smad4, Akt, TGF-B, and PTEN or portions thereof, co-immunoprecipitating the agent and AR, and assaying the interaction via competition assays wherein the activity of the agent on AR is decreased through the presence of a competitive inhibitor comprising the binding domain of AR

68. Disclosed are methods of detecting a effect of an agent on AR activity comprising assaying luciferase activity via the MMTV-luc reporter gene, wherein the cells used to measure the activity are LNCaP cells.

69. Disclosed are methods of identifying a modulator of the PI3K/Akt to AR pathway comprising, assaying the effects on AR activity using MMTV-luc and (ARE)4-luc reporter genes and altering the effect of the modulator through the use of an agent.

70. Disclosed are methods, wherein the agent is a constitutively active form of Akt, wherein the constitutively active form of Akt is cAkt, wherein the agent is a dominant negative form of Akt, wherein the dominant negative form of Akt is dAkt, wherein agent is an inhibitor of PI3K, wherein the inhibitor of PI3L is LY294002, wherein the agent is a constitutive active form of Akt, wherein the constitutively active form of Akt is cAkt, wherein the agent is a dominant negative form of Akt, wherein the dominant negative form of Akt is dAkt, wherein agent is an inhibitor of PI3K, wherein the inhibitor of PI3L is LY294002.

71. Disclosed are methods of blocking apoptosis by inhibiting the interaction of PNET with AR through the use of an agent, wherein, for example, the agent is ARf.

72. Disclosed are methods of identifying a compound that modulates androgen receptor activity comprising, a) administering a compound to a system, wherein the system has androgen receptor activity; b) assaying the effect of the compound on the amount of androgen receptor activity in the system; and c) selecting a compound which causes a change in the amount of androgen receptor activity in the system.

73. Also disclosed are methods of modulating androgen receptor activity comprising administering a compound, wherein the compound causes modulation of androgen receptor activity, wherein the compound is defined as a compound capable of being identified by administering the compound to a system, wherein the system has androgen receptor activity; and assaying the effect of the compound on the amount of androgen receptor activity in the system; and selecting the compound if it causes a change in the amount of androgen receptor activity in the system.

74. Also disclosed are methods of modulating androgen receptor activity comprising administering a compound that causes an inhibition or an increase of an interaction between androgen receptor and a protein selected from the group consisting of Smad3, Smad4, Akt, TGF-B, and PTEN or fragment thereof. An inhibition of an interaction means any decrease in the amount of the androgen receptor and the protein which are considered bound together. For example, a decrease of androgen receptor/protein interaction can be at least a 1.5 fold, at least a 2 fold, at least a 2.5 fold, at least a 3 fold, at least a 3.5 fold, at least a 4 fold, at least a 4.5 fold, at least a 5 fold, at least a 6 fold, at least a 7 fold, at least a 8 fold, at least a 9 fold, at least at least a 10 fold, at least a 15 fold, at least a 20 fold, at least a 25 fold, at least a 30 fold, at least a 50 fold, at least a 100 fold, at least a 500 fold, at least a 1000 fold increase in the observed K_{d} (dissociation constant) of the androgen receptor and the protein in the presence of the compound, for example. Likewise an increase of androgen receptor/protein interaction can be at least a 1.5 fold, at least a 2 fold, at least a 2.5 fold, at least a 3 fold, at least a 3.5 fold, at least a 4 fold, at least a 4.5 fold, at least a 5 fold, at least a 6 fold, at least a 7 fold, at least a 8 fold, at least a 9 fold, at least at least a 10 fold, at least a 15 fold, at least a 20 fold, at least a 25 fold, at least a 30 fold at least a 50 fold, at least a 100 fold, at least a 500 fold, at least a 1000 fold decrease in the observed K_{d} (dissociation constant) of the androgen receptor and the protein in the presence of the compound, for example.

75. Disclosed are methods of making a composition capable of modulating androgen receptor activity comprising mixing an androgen receptor altering compound with a pharmaceutically acceptable carrier, wherein the compound is identified by administering the compound to a system, wherein the system has androgen receptor activity; and assaying the effect of the compound on the amount of androgen receptor activity in the system; and selecting the compound if it causes a change in the amount of androgen receptor activity in the system.

76. A system is any combination of cells or reagents that allows for the tested activity. A system typically comprises components, such as proteins or nucleic acids expressing proteins, or immobilized proteins, or reagents. For example, a system could be a cell, wherein the cell expresses specific nucleic acids that encode specific proteins. In some embodiments, a system could also be an in vitro system comprising proteins, which may, for example, be immobilized. A system could also be an in vivo system, such as a mouse, which expresses a specific protein or set of proteins. Typically a system will be a cell system, wherein the cell comprises a specific combination of proteins. A system can also be a system wherein the components of the system are inducible. For example, in a cell system, various proteins, for example, AR and Smad3 may be expressed from inducible promoters. The promoters could be the same or different inducible promoter or both or one could be a constitutive promoter. The systems can comprise any of the compounds or reagents or molecules disclosed herein.

77. Disclosed are methods of making a compound that modulates androgen receptor activity comprising, a) administering a compound to a system, wherein the system has androgen receptor activity; b) assaying the effect of the compound on the amount of androgen receptor activity in the system; and c) selecting a compound which causes a change in the amount of androgen receptor activity in the system, and d) synthesizing the compound.

78. Disclosed are methods, wherein the change is at least a 1.5 fold, at least a 2 fold, at least a 2.5 fold, at least a 3 fold, at least a 3.5 fold, at least a 4 fold, at least a 4.5 fold, at least a 5 fold, at least a 6 fold, at least a 7 fold, at least a 8 fold, at least a 9 fold, at least at least a 10 fold, at least a 15 fold, at least a 20 fold, at least a 25 fold, at least a 30 fold, at least a 50 fold, at least a 100 fold, at least a 500 fold, at least a 1000 fold change.

79. Disclosed are methods of modulating androgen receptor activity comprising administering a compound, wherein the compound is identified as changing the amount of androgen receptor activity in a system.

80. Also disclosed are methods, wherein the change in the amount of androgen receptor activity is a decrease in the activity, wherein the change in the amount of androgen receptor activity is an increase in the activity, wherein the activity is the cellular proliferation activity of androgen receptor, wherein the activity is the apoptotic activity of androgen receptor, wherein the activity is the transcription activation activity of androgen receptor, wherein the activity is the PSA altering activity of androgen receptor, wherein the compound is a compound disclosed herein.

81. Also disclosed are methods further comprising a protein which interacts with the androgen receptor, for example, wherein the protein is AR, Smad3, Smad4, TGFB, PTEN, and/or Akt.

82. Disclosed are methods of identifying a modulator of an interaction between androgen receptor and Smad3 comprising a) administering a compound to a system, wherein the system comprises Smad3 and androgen receptor, b) assaying the effect of the compound on a Smad3-androgen receptor interaction, and c) selecting a compound which modulates the Smad3-androgen receptor interaction.

83. Disclosed are methods of identifying a modulator of an interaction between androgen receptor and Smad4 comprising a) administering a compound to a system, wherein the system comprises Smad4 and androgen receptor, b) assaying the effect of the compound on a Smad4-androgen receptor interaction, and c) selecting a compound which modulates the Smad4-androgen receptor interaction.

84. Disclosed are methods of identifying a modulator of an interaction between androgen receptor and Akt comprising a) administering a compound to a system, wherein the system comprises Akt and androgen receptor, b) assaying the effect of the compound on a Akt-androgen receptor interaction, and c) selecting a compound which modulates the Akt-androgen receptor interaction.

85. Disclosed are methods of identifying a modulator of an interaction between androgen receptor and PTEN comprising a) administering a compound to a system, wherein the system comprises Smad3 and androgen receptor, b) assaying the effect of the compound on a PTEN-androgen receptor interaction, and c) selecting a compound which modulates the PTEN-androgen receptor interaction.

86. Disclosed are methods wherein the modulator is an inhibitor and methods wherein the modulator is an enhancer.

87. Disclosed are cells comprising, a) a regulatable nucleic acid comprising sequence encoding an AR gene and b) a nucleic acid comprising sequence encoding a Smad3 gene.

88. Disclsoed are cells comprising, a) a regulatable nucleic acid comprising sequence encoding an AR gene and b) a nucleic acid comprising sequence encoding a Smad4 gene.

89. Disclosed are cells comprising, a) a regulatable nucleic acid comprising sequence encoding an AR gene and b) a nucleic acid comprising sequence encoding a Akt gene.

90. Also disclosed are cells comprising, a) a regulatable nucleic acid comprising sequence encoding an AR gene and b) a nucleic acid comprising sequence encoding a PTEN gene.

91. Disclosed are cells comprising, a) a regulatable nucleic acid comprising sequence encoding an AR gene and b) a nucleic acid comprising sequence encoding a TGF-B gene.

92. It is understood that cells that comprise at least one of the proteins disclosed herein, such as AR, Smad3, Smad4, TGF-B, PTEN, or Akt, or fragments or a combination of these proteins or fragments, are considered systems as disclosed herein. It is understood that these proteins can be expressed from transgenic nucleic acids or from native nucleic acids. It is also understood that in the systems, the nucleic acids expressing encoding the proteins may be regulatable or constitutive as described herein.

C. Compositions

93. Disclosed are compositions and methods, which are related to androgen receptor signal transduction pathways. The disclosed pathways and interactions are involved in modulating androgen receptor effects on cells, such as prostate cells, and as disclosed herein can be modulated by the disclosed compositions. In general the compositions disclosed are compositions, which can modulate one or more of the signal transduction pathways related to androgen receptor. Also disclosed are various compositions involved in the signal transduction pathways related to androgen receptor, and these compositions are useful, for example, as targets in screening assays for modulators of androgen receptor pathways and functional effects.

1. Androgen Receptor

AR is a phosphoprotein, and the consensus phosphorylation sites found in AR indicated that AR could be a substrate for the DNA-dependent protein kinase, protein kinase A (PKA), protein kinase C (PKC), mitogen-activated kinase (MAPK), and casein kinase II (Blok et al. (1996) Endocr Res 22, 197-219). This hypothesis was supported by the observation that PKA and PKC could enhance AR transactivation (Ikonen et al. (1994) Endocrinology 135, 1359-66; Nazareth et al. (1996) J Biol Chem 271, 19900-7). Furthermore, a report also demonstrated that the HER2/Neu-MAPK pathway could phosphorylate AR that might result in much easier recruitment of AR coregulators to AR. The consequence of this signal cascade may then enhance AR transactivation (Yeh et al. (1999) Proc Natl Acad Sci USA 96, 5458-63).

94. In addition to stimulating cell growth, androgen/AR plays important roles in the promotion of cell apoptosis. For examples, androgen can induce the thymic atrophy by acceleration of thymocyte apoptosis (Olsen et al. (1998) Endocrinology 139, 748-52). Androgen also causes the biphasic growth (stimulation of cell growth at 10-12-10-10M and suppression of cell growth at 10-8M) in the prostate cancer LNCaP cells, which expresses functional AR (Zhao et al. (1999) Endocrinology 140, 1205-12). AR also plays indispensible roles in the mitogen-activated protein kinase kinase kinase-1 (MAPKKK1)-induced apoptosis in the prostate cancer cells (Abreu-Martin et al. (1999) Mol Cell Biol 19, 5143-54). Androgen also induces cell growth inhibition and apoptosis in the PC-3(AR)2 with stably transfected AR (Heisler et al. (1997) Mol Cell Endocrinol 126, 59-73). Finally, the tumor suppressor BRCA-1 increases the AR transactivation and promotes the androgen-induced cell death (Park et al. (2000) Cancer Res. 60, 5946-9; Yeh et al. (2000) Proc Natl Acad Sci USA 97, 11256-61). Taken together, it is well documented that androgen/AR may play dual roles in the promotion of cell growth and apoptosis.

95. The androgen receptor (AR), a member of the steroid receptor superfamily, functions as an androgen-dependent transcriptional factor (Chang et al. (1988) Science 240, 324-326). After bindinig to ligand, the activated AR is able to recognize palindromic DNA sequences, called androgen response elements (AREs), and form a complex with AR associated proteins to induce the expression of AR target genes. Several AR coregulators (ARAs), such as ARA24, ARA54, ARA55, ARA70, ARA160, ARA267, Rb, BRCA1 and TIFIIH, have been isolated and characterize (Hsiao et al. (1999) J. Biol. Chem. 274, 22373-22379; Kang et al. (1999) J. Biol. Chem. 274, 8570-8576; Fujimoto et al. (1999) J. Biol. Chem. 274, 8316-8321; Yeh et al. (1996) Proc. Natl. Acad. Sci. USA 93, 5517-5521; Hsiao et al. (1999) J. Biol. Chem. 274,20229-20234; Yeh et al. (1998) Biochem. Biophys. Res. Commun. 248, 361-367; Yeh et al. (2000) Proc. Natl. Acad. Sci. USA 97, 11256-11261; Lee et al. (2000) J. Biol. Chem. 275, 9308-9313). Results from these studies suggest that coregulators not only can enhance AR transactivation, but may also be able to increase the agonist activity of antiandrogens (Miyamoto et al. (1998) Proc. Natl. Acad. Sci. USA 95, 7379-7384) and 17-β estradiol (Yeh et al. (1998) Proc. Natl. Acad. Sci. USA 95, 5527-5532:) in prostate cancer DU145 cells.

96. Sequence analysis of AR reveals two Akt consensus sequences (RXRXXS/T) (Alessi et al. (1996) FEBS Lett 399, 333-8) located at its amino-terminal domain and caxboxyl-terminal domain (Ser210 (RAREAS) and Ser790 (RMRHLS)).

97. Herein it is disclosed that Akt phosphorylates AR at Ser210, inhibits AR transactivation, and blocks AR-induced apoptosis.

98. Disclosed herein both Smad3 and Smad4 can interact with AR in the DNA-binding domain (DBD) and the ligand-binding domain (LBD). Also disclosed Smad4 can decrease the AR-Smad3 interaction and repress the Smad3enhanced AR transactivation. These inhibitory functions of Smad3/Smad4 on AR transactivation in the prostate cancer cells indicate that Smad4 can cooperate with Smad3 to modulate AR transactivation.

2. Phosphophatidylinositol 3(OH)-kinase (PI(3)K)

99. Phosphophatidylinositol 3(OH)-kinase (PI(3)K) contains the p85 regulatory domain and p110 catalytic domain. The p85 regulatory domain possesses two src-homology 2 (SH2) domains and a src-homology 3 (SH3) domain. The major role of the SH2 domain is to facilitate tyrosine kinase-dependent regulation of PI(3)K activity by increasing the catalytic activity of p110 and by inducing the recruitment of PI(3)K to the signaling complex (Carpenter et al. (1996) Biochim Biophys Acta 1288, M11-6). PI(3)K phosphorylates the inositol ring of PI(4,5)P2 at the D-3 position to form PI(3,4,5) P3. This lipid product of PI(3)K then activates Akt/Protein kinase B (PKB) in the membrane.

3. Akt/PKB

Akt/PKB, an oncoprotein, is a serine (Ser)-threonine (Thr) protein kinase. The amino terminus of Akt/PKB contains a pleckstrin homology domain, which could bind to the lipid products of PI(3)K (Franke et al. (1997) Cell 88,435-7). Phosphorylation of Akt/PKB at Thr308 and Ser473 results in full activation of Akt/PKB kinase activity (Chan, et al. (1999) Annu Rev Biochem 68, 965-1014). The PI(3)K/Akt pathway in diverse cell types provides the survival signal that involves several proapoptotic proteins such as Bad (Datta et al. (1997) Cell 91, 231-41; del Peso et al. (1997) Science 278, 687-9) and Caspase-9 (Cardone et al. (1998) Science 282, 1318-21).

100. Data indicate that the role of Akt/PKB is to function as a general mediator of cell survival. Franke, T. F. et al., Cell 88,435-437 (1997). Several growth factors, such as insulin-like growth factor 1 and neurotrophins, may promote cell survival by activating the PI3K and its downstream target Akt/PKB. Akt/PKB may then phosphorylate and inhibit pro-apoptotic components, such as BAD, Caspase-9 and FKHRLI. Dana, S. R et al., Cell 91, 231-241 (1997); Cardone, M. H. et al., Science 282, 1318-1321 (1998); Brunet, A. et al., Cell 96, 857-868 (1999). Disclosed herein, Akt/PKB phosphorylates AR.

101. Disclosed herein, Akt phosphorylates the androgen receptor (AR) at Ser210 and Ser790. A mutation at AR Ser210 results in the reversal of Akt-mediated suppression of AR transactivation. Activation of the phosphatidylinositol-3-OH kinase/Akt pathway results in the suppression of AR target genes, such as p21, and the decrease of androgen/AR-mediated apoptosis, through the inhibition of interaction between AR and AR coregulators. Disclosed is the molecular basis for cross-talk between two signaling pathways at the level of Akt and AR-AR coregulators.

4. Interleukin-6 (“IL-6”)

102. Interleukin-6 (“IL-6”) is a pleiotropic cytokine that has been associated with the growth of many tumor cells. Hobisch, A. et al., Cancer Res. 58, 4640-4645 (1998). Whether IL-6 stimulates or inhibits prostate cancer growth was controversial. Qui. Y. et al., Nature 393, 83-85 (1998); Hobisch, A et al., Cancer Res. 58, 4640-4645 (1998); Ritchie, C. K. et al., Endocrinology 138,1145-1150 (1997). Specific signaling events that link IL-6 to the androgen-AR signal transduction pathway is largely unexplored.

103. IL-6 has two major signal cascades: mitogen-activated protein kinase (“MAPK”) and phosphatidylinositol 3-kinase (“PI3K”). Qui, Y. et al., Nature 393,83-85 (1998); Qui, Y. et al., Proc. Natl. Acad. Sci. USA 95, 3644-3649 (1998). With the MAPK cascade, IL-6 induces MAPK activation. Qui, Y. et al., Nature 393, 83-85 (1998). It has been shown that when MAPK is activated by overexpression of the HER2/Neu protooncogene, AR transactivation is increased. Yeh, S. et al., Proc. Natl. Acad. Sci. USA 96, (1999). With the PI3K cascade, IL-6 induces PI3K kinase activity. Qui, Y. et al., Proc. Natl. Acad. Sci. USA 95, 3644-3649 (1998). PI3K has two major downstream effectors: a serine/threonine kinase (“Akt/PKB”) and a ribosomal S6 kinase (“p70S6k”). Franke, T. F. et al., Cell 88,435-437 (1997).

5. Transforming Growth Factor β (TGF-β)

104. Transforming growth factor β (TGF-β) signaling is mediated through two types of transmembrane serine/theonine kinase receptors (Massague. (1996) Cell 85, 947-50). Upon binding to TGF-β, the type II TGF-β receptor (TβRII) forms a heteromeric complex with the type I TGF-β receptor (TβRI), resulting in the phosphorylation and activation of TβRI (Wrana et al. (1994) Nature 370, 341-7). The activated TβRI then interacts with an adaptor protein SARA (Smad anchor for receptor activation) (Tsukazaki et al. (1998) Cell 95, 779-91), which propagates signals to intracellular signaling mediators known as Smad2 and Smad3 (Derynck et al. (1998) Cell 95, 73740.). Following association with Smad4, the Smad complexes translocate to the nucleus where they activate specific target genes through cooperative interactions with DNA and other DNA-binding proteins such as FAST1 and Fos/Jun (AP-1) (Chen et al. (1996) Nature 383, 691-6; Zhang et al. (1998) Nature 394, 909-13).

TGF-β plays a dual role in tumorigenesis. On the one hand, TGF-β inhibits the growth of normal epithelial and endothelial cells (Massague (1990) Annu Rev Cell Biol 6, 597-641) and induces cell-cycle inhibitors such as p15^{INK4B} and p21 ^{WAF1/CIP} (Hannon et al. (1994) Nature 371, 257-61; Attisano et al. (1994) Biochim Biophys Acta 1222, 71-80). On the other hand, TGF-β can accelerate the malignant process during late stages of tumorigenesis (Barrack (1997) Prostate 31, 61-70; Cui et al. (1996) Cell 86, 531-42). TGF-β is abundantly expressed in various tumors of epithelial origin (Derynck et al. (1985) Nature 316, 701-5) in which it can suppress immune surveillance (Letterio et al. (1998) Annu Rev Immunol 16, 137-61), facilitate tumor invasion (Cui et al. (1996) Cell 86, 531-42), and promote the development of metastases (Yin et al. (1999) J Clin Invest 103, 197-206). The study of TGF-β expression indicates that it may be involved in the development of prostate cancer in animal models (Thompson et al. (1993) Cancer 71, 1165-71). Moreover, plasma TGF-β was significantly elevated in patients with clinically evident metastases and correlated with increasing serum prostate specific antigen (PSA) levels (Ivanovic et al. (1995) Nat Med 1, 282-4; Adler et al. (1999) J Urol 161, 182-7).

105. Disclosed herein, Smad3, a downstream mediator of the TGF-β signaling pathway, functions as a coregulator to enhance androgen receptor (AR)-mediated transactivation. Aslo disclosed herein, as compared to wild type AR, Smad3 acts as a strong coregulator in the presence of 1 nM 5α-dihydrotestosterone, 10 nM 17β-estradiol, or 1 μM hydroxyflutamide for the LNCaP mutant AR (mtAR T877A), found in many prostate tumor patients. Aslo disclosed is that endogenous PSA expression in LNCaP cells can be induced by 5α-dihydrotestosterone and the addition of the Smad3 further induces PSA expression. Disclosed herein is that Smad3 is a coregulator for the androgen-signaling pathway as well as the involvement of TGF-β in the androgen-promoted prostate cancer growth.

6. PTEN (Phosphatase and Tensin Homolog Deleted on Chromosome Ten)

106. The tumor suppressor gene PTEN (phosphatase and tensin homolog deleted on chromosome ten), located at chromosome 10q23, is one of the most frequently mutated genes linked to a variety of human cancers (Li et al. (1997) Science 275, 1943-7; Davies et al. (1999) Cancer Res 59, 2551-6; Cantley et al. (1999) Proc Natl Acad Sci USA 96, 4240-5; Di Cristofano et al. (2000) Cell 100, 387-90; Feilotter et al. (1999) Br J Cancer 79, 718-23; Feilotter et al. (1998) Oncogene 16, 1743-8; Steck et al. (1997) Nat Genet 15, 356-62). Germline mutations in PTEN cause the autosomal dominant inherited cancer syndromes such as Cowden disease, which is associated with an elevated risk for malignant cancers (Liaw et al. (1997) Nat Genet 16, 64-7). Loss of PTEN expression is frequently found in prostate cancer cell lines and tumor specimens (Vlietstra et al. (1998) Cancer Res 58, 2720-3). Mice with a heterozygous mutant PEN develop prostate epithelial hyperplasia and dysplasia (Di Cristofano et al. (1998) Nat Genet 19, 348-55). Mice with inactivation of one allele of PTEN in combination with loss of the CDKn1b (encoding p27^{Kip1}) gene have an acceleration of spontaneous neoplastic transformation and develop prostate carcinoma (Di Cristofano et al. (2001) Nat Genet 27, 222-4). Interestingly, mice deficient in CDkn1b do not develop prostate cancer (Kiyokawa et al. (1996) Cell 85, 721-32; Fero et al. (1996) Cell 85, 733-44; Nakayama et al. (1996) Cell 85, 707-20), suggesting that PTEN and p27^{Kip1} cooperate in prostate cancer suppression in the mouse. These results indicate that loss of PTEN function may be a key event in prostate cancer progression.

107. Recent studies demonstrated that PTEN regulates not only cell growth and apoptosis, but also controls cell adhesion, migration via regulating the focal adhesion kinase, and shc activity (Tamura et al. (1998) Science 280, 161-47; Tamura et al. (1999) J Natl Cancer Inst 91, 1820-8; Gu et al. (1999) J Cell Biol 146, 389-403). While the PTEN sequence suggests that it may be a dual specificity phosphatase, its protein substrates remain largely unknown. Recently, several groups have reported that the phosphatidylinositol-3-OH kinase (PI3K)/Akt pathway is negatively regulated by PTEN through its phospholipid 3-phosphatase activity (Cantley et al. (1999) Proc Natl Acad Sci USA 96, 4240-5; Di Cristofano et al. (2000) Cell 100, 387-90; Maehama et al. (1998) J Biol Chem 273, 13375-8; Maehama et al. (1999) Trends Cell Biol 9, 125-8; Hopkin, K. (1998) Science 282, 1027,1029 -30). The PI3K/Akt-dependent pathway is the most popular model for PTEN action, however, signaling pathways other than PI3K/Akt are also suggested (Gao et al. (2000) Dev Biol 221, 404-18).

108. Disclosed herein AR interacts with the phosphatase domain of PTEN. Also disclosed herein, interaction between PTEN and AR promotes AR protein degradation that results in the suppression of AR transactivation and induction of apoptosis. Also disclosed is a minimum interaction peptide within AR (aa, 483-651) disrupts the interaction of PTEN with AR and reduces PTEN effect on AR transactivation and apoptosis. In addition, the phosphatase domain within PTEN can mimic the effects of PTEN on the suppression of AR activity. Also disclosed, the interactions between PTEN and AR contribute to PTEN-induced suppression of AR functions and apoptosis.

7. Smads

109. Smads are a class of proteins that function as central mediators of the transforming growth factor β (TGF-β) superfamily (Derynck et al. (1998) Cell 95,737-740; Massagué (1998) Ann. Rev. of Biochem. 67, 753-791). Smads are directly phosphorylated and activated by type I TGF-β family receptors (Zhang et al. (1996) Nature 383, 168-72; Macias-Silva et al. (1996) Cell 87, 1215-1224). TGF-β and activin receptors phosphorylate Smad2 and Smad3 (Zhang et al. (1996) Nature 383, 168-72; Nakao et al. (1997) Embo. J. 16, 5353-5362), whereas bone morphogenetic protein receptors phosphorylate Smads 1, 5, and 8 (Liu et al. (1996) Nature 381, 620-623; Graff et al. (1996) Cell 85, 479-487; Hoodless et al. (1996) Cell 85, 489-500). Upon phosphorylation, these Smads will associate with Smad4 and move into the nucleus where they assemble transcriptional complexes that activate specific sets of genes. Thus, Smad4 is a shared key component of these various signaling pathways. A distinct structural feature that distinguishes Smad4 from other Smads is the lack of the SSXS motif at the tail of the Mad homology 2 (MH2) domain terminal that can be phosphorylated by the cognate receptor kinases (Zhang et al. (1996) Nature 383, 168-72; Macias-Silva et al. (1996) Cell 87, 1215-1224; Kretzschmar et al. (1997) Genes Dev. 11, 984-995; Liu et al. (1997) Proc. Natl. Acad. Sci. USA 94, 10669-10674).

110. Smad4 was originally identified as a candidate tumor suppressor gene in chromosome 18q21 that was somatically deleted/mutated/inactivated in many pancreatic or colorectal tumors (Hahn et al. (1996) Science 271, 350-3; Howe et al. (1998) Science 280, 1086-1088; Thiagalingam et al. (1996) Nat. Genet. 13, 343-346). Knock-out smad4studies indicated that the Smad4-null mouse has early embryonic lethality (Takaku et al. (1998) Cell 92, 645-656; Sirard et al. (1998) Genes Dev. 12, 107-119). The introduction of the Smad4 gene into Smad4-null cells also suggested that the wild type (wt) Smad4 could decrease the cell growth rate, cause a cell cycle arrest, and induce apoptosis (Dai et al. (1999) Proc. Natl. Acad. Sci. USA 96, 1427-1432).

111. Although Smad4 has been reported to be infrequently mutated or deleted in breast (Schutte et al. (1996) Cancer Res. 56, 2527-2530), ovarian (Schutte et al. (1996) Cancer Res. 56, 2527-2530), and prostate cancers (MacGrogan et al. (1997) Oncogene 15, 1111-1114), a significant increase of Smad4 was observed in the normal ventral prostate, as well as in the prostate tumors after castration. Moreover, a previous report has shown that the stining for Smad4 was present in areas with a large number of apoptotic cells in the prostate after castration (Brodin et al. (1999) Cancer Res. 59, 2731-2738). Smad4 proteins have been found to functionally interact with Smad3 (Zhang et al. (1996) Nature 383, 168-72), AP-1 (Zhang et al. (1998) Nature 394, 909-913), and p300/CBP (Feng et al. (1998) Genes Dev. 12, 2153-2163), it is possible that Smad4 may also play an important role in the modulation of the androgen-mediated signal pathway.

112. Disclosed herein the Smad4, together with Smad3, and each individually, can interact with the androgen receptor (AR) in the DNA-binding and ligand-binding domains, which results in the modulation of 5α-dihydrotestosterone-induced AR transactivation. Disclosed herein in prostate PC-3 and LNCaP cells, addition of Smad3 alone can induce AR transactivation and co-transfection of Smad3/Smad4 can then repress AR transactivation in various androgen response element-promoter reporter assays as well as Northern blot and RT-PCR quantitation assays with prostate specific antigen mRNA expression. In contrast, in the SW480·C7 cells, without an endogenous functional Smad4, the influence of Smad3 on AR transactivation is dependent on the various androgen response element-promoters. Disclosed herein, the Smad3/Smad4 influence on AR transactivation involves acetylation since treatment of trichostatin A or NaB can reverse Smad3/Smad4-repressed AR transactivation and the Smad3/Smad4 complex can also decrease the acetylation level of AR. Disclosed herein, homodimers or heterodimers between Smad3 and Smad4, as well as their interactions with AR, result in the differential regulation of AR transactivation.

8. Sequence Similarities

113. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

114. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

115. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL BioL 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

116. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

117. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

9. Hybridization/selective Hybridization

118. The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

119. Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, t,he conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

120. Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

121. Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75,76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the pruner molecules are extended. Preferred conditions also include those suggested by the manufacturer or indicated in the art as being appropriate for the enzyme performing the manipulation.

122. Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

123. It is understood that those of skill in the art understand that if a composition or method meets any one of these criteria for determining hybridization either collectively or singly it is a composition or method that is disclosed herein.

10. Nucleic Acids

124. There are a variety of molecules disclosed herein that are nucleic acid based, including for example the nucleic acids that encode, for example AR, Smad3, Smad4, TGF-B, Akt, IL-6, and PTEN, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

125. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

126. A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

127. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

128. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556). There are many varieties of these types of molecules available in the art and available herein.

129. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

130. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

131. There are a variety of sequences related to the protein molecules involved in the signaling pathways disclosed herein, for example, AR, Smad3, Smad4, TGF-B, Akt, IL-6, and PTEN, all of which are encoded by nucleic acids. The sequences for the human analogs of these genes, as well as other anlogs, and alleles of these genes, and splice variants and other types of variants, are available in a variety of protein and gene databases, including Genbank (for example Genbank accession numbers NM_{—}000044 (SEQ ID NO: 1), L49399 (SEQ ID NO: 2), AH011390 (SEQ ID NO: 3), SEG_AB004922S (SEQ ID NO: 4), AB043547 (SEQ ID NO: 5), E00973 (SEQ ID NO: 6), XM_{—}081482 (SEQ ID NO: 7), AH007803 (SEQ ID NO: 8), AH005966 (SEQ ID NO: 9), AF067844 (SEQ ID NO: 10), and AF372214 (SEQ ID NO: 11)). Those sequences available at the time of filing this application at Genbank are herein incorporated by reference in their entireties as well as for individual subsequences contained therein Genbank can be accessed at http://www.ncbi.nih.gov/entrez/guery.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

132. Disclosed are compositions including primers and probes, which are capable of interacting with the genes of the disclosed proteins as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription Techniques and conditions that amplify the primer in a sequence specific manner are preferred. In certain embodiments the primers are used for the DNA amplification reactions, such as PCR or direct sequencing. It is understood that in certain embodiments the primers can also be extended using non enzymatic techniques, where for example, the nucleotides or oligonucleotides used to extend the primer are modified such that they will chemically react to extend the primer in a sequence specific manner. Typically the disclosed primers hybridize with the gene or region of the gene or they hybridize with the complement of the gene or complement of a region of the gene. The size of the primers or probes can be any size that supports the desired enzymatic manipulation of the primer, such as DNA amplification o rthe simple hybridization of the probe or primer. The products produced by enzymatic reactions dependent on the primers can be any size, but typically would be less than 4000 bases long.

d) Functional Nucleic Acids

133. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

134. Functional nucleic acid molecules can interact with any macromolecule, such as DNA, RNA, polypeptides, or carbohydrate chains. Thus, functional nucleic acids can interact with the mRNA of the disclosed polypeptides, such as AR, Smad3, Smad4, TGF-B, Akt, IL-6, and PTEN, or the genomic DNA of the disclosed polypeptides, such as AR, Smad3, Smad4, TGF-B, Akt, IL-6, and PTEN, or they can interact with the polypeptides themselves, such as AR, Smad3, Smad4, TGF-B, Akt, IL-6, and PTEN. Also disclosed are molecules that interact with fragments, such as functional or antigeneic fragments of the disclosed molecules, such as AR, Smad3, Smad4, TGF-B, Akt, IL-6, and PTEN Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

135. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than 10⁻⁶. It is more preferred that antisense molecules bind with a k_{d} less than 10⁻⁸. It is also more preferred that the antisense molecules bind the target moelcule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos.: 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

136. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of aptamers polypeptides, such as AR, Smad3, Smad4, TGF-B, Akt, IL-6, and PTEN, the background protein could be serum albumin. Representative examples of how to make and use aptamers to bind a variety of different target molecules can be found in the following non-limiting list of U.S. Pat. Nos.: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

137. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos.: 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos.: 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos.: 5,595,873 and 5,652,107). There are also a number of ribozymes that are not found in natural systems, but which have been engineered to catalyze specific reactions de novo (for example, but not limited to the following U.S. Pat. Nos.: 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos.: 5,646,042, 5,693,535, 5,731,295, 5,811,300, 5,837,855, 5,869,253, 5,877,021, 5,877,022, 5,972,699, 5,972,704, 5,989,906, and 6,017,756.

138. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target moelcule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos.: 5,176,996, 5,645,985, 5,650,316, 5,683,874, 5,693,773, 5,834,185, 5,869,246, 5,874,566, and 5,962,426.

139. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and FoFster and Altman, Science 238:407-409 (1990)).

140. Similarly, eukaryotic EGS/RNAse P-directed cleavage of RNA can be utilized to cleave desired targets within eukarotic cells. (Yuan et al., Proc Natl Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos.: 5,168,053, 5,624,824, 5,683,873, 5,728,521, 5,869,248, and 5,877,162

11. Peptides

a) Protein Variants

141. As discussed herein there are numerous variants of the disclosed proteins, such as, AR, Smad3, Smad4, TGF-B, Akt, IL-6, PTEN that are known and herein contemplated. In addition, to the known functional allelic variants there are derivatives of the disclosed proteins, such as AR, Smad3, Smad4, TGF-B, Akt, IL-6, PTEN which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | |
|---|---|
| alanine | AlaA |
| allosoleucine | Aile |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2

Amino Acid Substitutions
Original Residue Exemplary Conservative
Substitutions, others are known in the art.

| | |
|---|---|
| Ala | ser |
| Ar | glys, gln |
| Asn | gln; his |
| Asp | glu |
| Cys | ser |
| Gln | asn, lys |
| Glu | asp |
| Gly | ala |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

143. Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

144. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

145. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

146. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

147. It is understood that one way to define the variants and derivatives of the disclosed proteins herein is through defining the variants and derivatives in terms of homology/identity to specific known sequences. Specifically disclosed are variants of the disclosed polypeptides, such as AR, Smad3, Smad4, TGF-B, Akt, IL-6, PTEN and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

148. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

149. The same types of homology can be obtained for nucleic acids by for example the algorithms disclosed in Zuker, M. Science 244:48-52, 1989, Jaeger et al. Proc Natl Acad Sci USA 86:7706-7710, 1989, Jaeger et al. Methods Enzymol 183:281-306, 1989 which are herein incorporated by reference for at least material related to nucleic acid alignment.

150. It is understood that the description of conservative mutations and homology can be combined together in any combination, such as embodiments that have at least 70% homology to a particular sequence wherein the variants are conservative mutations.

12. Antibodies

(1) Antibodies Generally

151. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the proteins disclosed herein, such as AR, Smad3, Smad4, TGF-B, Akt, IL-6, and PTEN, such that the antibody can modulate the effect of androgen receptor mediated events. Antibodies that bind the disclosed regions of the disclosed polypeptides, such as AR, Smad3, Smad4, TGF-B, Akt, IL-6, PTEN involved in the disclosed signal transduction pathways related to the AR are also disclosed. The antibodies can be tested for their desired activity using the in vitro assays described herein, or by analogous methods, after which their in vivo therapeutic and/or prophylactic activities are tested according to known clinical testing methods.

152. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light-chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc Natl Acad Sci USA, 81:6851-6855 (1984)).

153. The disclosed monoclonal antibodies can be made using any procedure which produces mono clonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent.

154. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

155. In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

156. The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment (Zoller, M. J. Curr. Opin. Biotechnol. 3:348-354, 1992).

157. As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

(2) Human Antibodies

158. The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J Immunol, 147(1):86-95, 1991). Human antibodies of the invention (and fragments thereof) can also be produced using phage display libraries (Hoogenboom et al., J Mol Biol, 227:381, 1991; Marks et al., J Mol Biol, 222:581, 1991).

159. The human antibodies of the invention can also be obtained from transgenic animals. For example, transgenic, mutant mice that are capable of producing a full repertoire of human antibodies, in response to immunization, have been described (see, e.g., Jakobovits et al., Proc Natl Acad Sci USA, 90:2551-255 (1993); Jakobovits et al., Nature, 362:255-258 (1993); Bruggermann et al., Year in Inmunol, 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge.

(3) Humanized Antibodies

160. Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

161. To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

162. Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

163. Administration of the antibodies can be done as disclosed herein. Nucleic acid approaches for antibody delivery also exist. The broadly neutralizing anti such as AR, Smad3, Smad4, TGF-B, Akt, IL-6, and PTEN antibodies or fragments thereof and antibody fragments of the invention can also be administered to patients or subjects as a nucleic acid preparation (e.g., DNA or RNA) that encodes the antibody or antibody fragment, such that the patients or subjects own cells take up the nucleic acid and produce and secrete the encoded antibody or antibody fragment. The delivery of the nucleic acid can be by any means, as disclosed herein, for example.

(5) Diagnostic and Cytotoxic Agents

164. The antibodies the disclosed polypeptides, such as AR, Smad3, Smad4, TGF-B, Akt, IL-6, and PTEN or fragments thereof can be used to identify and/or inactivate cancer cells that are dependent on the proteins involved in the disclosed signal transduction pathways in vitro or in vivo. Typically, the antibody or analog will be coupled to a label which is detectable but which does not interfere with binding to the disclosed polypeptides, such as AR, Smad3, Smad4, TGF-B, Akt, IL6, and PTEN or fragment thereof. Although described primarily with reference to radioisotopes, especially indium (“In”), which is useful for diagnostic purposes, and yttrium (“Y”), which is cytotoxic, other substances which harm or inactivate cancer cells can be substituted for the radioisotope. The antibodies or substrate analogs may be unlabeled or labeled with a therapeutic agent. These agents can be coupled either directly or indirectly to the disclosed antibodies or substrate analogs. One example of indirect coupling is by use of a spacer moiety. These spacer moieties, in turn, can be either insoluble or soluble (Diener, et al., Science, 231:148, 1986) and can be selected to enable drug release from the antibodies or substrate analogs at the target site. Examples of therapeutic agents which can be coupled to the disclosed antibodies or substrate analogs are drugs, radioisotopes, lectins, and toxins or agents which will covalently attach the antibody or substrate analog to the mema.

165. Certain isotypes may be more preferable than others depending on such factors as distribution as well as isotype stability and emission. In general, alpha and beta particle-emitting radioisotopes are preferred in immunotherapy. Preferred are short range, high energy alpha emitters such as ²¹²Bi. Examples of radioisotopes which can be bound to the disclosed antibodies for therapeutic purposes are. ¹²⁵I, ¹³¹I, ⁹⁰Y, ⁶⁷Cu, ²¹²Bi, ²¹¹At, ²¹²Pb, ⁴⁷Sc, ¹⁰⁹Pd, and ¹⁸⁸Re.

166. Toxins are poisonous substances produced by plants, animals, or microorganisms that, in sufficient dose, are often lethal. Diphtheria toxin is a substance produced by Corynebacterium diphtheria which can be used therapeutically. This toxin consists of an alpha and beta subunit which under proper conditions can be separated. Lectins are proteins, usually isolated from plant material, which bind to specific sugar moieties. Many lectins are also able to agglutinate cells and stimulate lymphocytes. However, ricin is a toxic lectin which has been used immumotherapeutically. This is accomplished by binding the alpha-peptide chain of ricin, which is responsible for toxicity, to the antibody molecule to enable site specific delivery of the toxic effect. Other therapeutic agents which can be coupled to the disclosed antibodies are known, or can be easily ascertained, by those of ordinary skill in the art.

167. The radioisotopes are preferred since they are small and well characterized, and can be used as diagnostics and followed after administration using standard non-invasive radioimaging techniques.

168. As radioisotopes decay, they emit characteristic photons or particles or both. Photons, commonly referred to as gamma rays, are penetrating. If their energy level is high enough, they can travel through the body and be detected by diagnostic instrumentation. Radioisotopes that emit photons can be attached to an antibody or substrate analog and used for diagnostic imaging. This application is termed radioimmunoscintigraphy (RIS). The shorter the distance between the antigen and the target, the shorter the required range of emission of the radioisotope. Auger electrons have a very short path length (5-10 nm) and need to be internalized to be cytotoxic (Adelstein, et al., Nucl. Med. Biol. 14:165-169 (1987)). Only antibodies or substrate analogs that are internalized after binding to a cell should be considered for radioisotopes that emit Auger electrons. Alpha particles need to be close to a cell (within 3-4 cell diameters) to be effective (Vriesendorp, et al., Radioimmunoglobulin therapy. In: High Dose Cancer Therapy. Armitage, et al. (eds). (Williams & Wilkins, Baltimore, Md. 1992 pp. 84-123). Both Auger electrons and alpha emitters have high selectivity because their short-range emission will not irradiate neighboring normal cells.

169. The radiometals ¹¹¹In and ⁹⁰Y are, respectively, pure γ- and pure β-emitters. Iodine-125, the most commonly used emitter of Auger electrons, has a half-life of 60 days and fluently is released by the immunoconjugate in vivo (dehalogenation) (Vriesendorp, et al., 1992). The most commonly considered alpha emitters for clinical use, astatine-211 and bismuth-212, have short half-lives (7.2 h and 1.0 h, respectively) and decay into radioactive isotopes, that may not be retained by the immunoconjugate after the first alpha emission (Wilbur, Antibiot. Immunoconjug. Radiopharm. 4:85-97 (1991)). The use of an immunoconjugate radiolabeled with ¹¹¹In has been proposed to predict the behavior of the poorly imageable ⁹⁰Y-labeled immunoconjugate (Korngold, et al., Cancer Res. 20:1488-1494 (1960); Welt, et al., J. Clin. Oncol. 12:1561-1571 (1994); Breitz, et al., J. Nucl. Med. 33:1099-1112 (1992); Vriesendorp, et al., Cancer Res. (suppl) 55:5888s-5892s (1995)). Previous studies using stable radiometal chelation have demonstrated similar biodistributions for radioimmunoconjugates labeled with ¹¹¹In and ⁹⁰Y (Welt, et al., J. Clin. Oncol. 12:1561-1571 (1994); Breitz, et al., J. Nucl. Med. 33:1099-1112 (1992)).

170. For diagnostic administration, the immunoconjugate would be radiolabeled with a pure gamma-emitting radioisotope like indium-111 (¹¹¹In) or technetium-99m (^{99m}Tc). Both of these isotopes emit gamma rays within the appropriate energy range for imaging 100-250 keV). Energies below this range are not penetrating enough to reach an external imaging device. Higher energy levels are difficult to collimate and provide diagnostic images with poor resolution. The short-half life of ^{99m}Tc restricts its use to immunoconjugates with rapid tumor uptake. The use of ¹¹¹In-labeled immunoconjugate has been proposed to predict the in vivo behavior of an immunoconjugate radiolabeled with ⁹⁰Y, a pure beta-emitter, since they have similar half-lives and comparable chelation chemistry (Vriesendorp, et al., Cancer. Res. (suppl) 55:5888s-5892s (1995); Vriesendorp, et al., Radioimmunoglobulin therapy. 1992); DeNardo, et al., J. Nucl. Med. 36:829-836 (1995); Leichner, et al., Int. J. Radiat. Oncol. Biol. Phys. 14:1033-1042 (1988)). An advantage of using two separate radioisotopes, one for imaging and one for therapy, is that it allows for outpatient treatment. The low amount of radioactivity used diagnostically does not represent a radiation hazard, while the radiation emitted by a therapeutic pure beta-emitter will largely be absorbed in the vicinity of the targeted cells. This treatment scheme is dependent on similar pharmacokinetics for both radiolabeled reagents and requires a stable means of attaching both radioactive compounds to the antibody.

171. Some radioisotopes can be attached directly to the antibody; others require an indirect form of attachment. The radioisotopes ¹²⁵I, ¹³¹I, ^{99m}Tc, ¹⁸⁶Re and ¹⁸⁸Re can be covalently bound to proteins (including antibodies) through amino acid functional groups. For radioactive iodine it is usually through the phenolic group found on tyrosine. There are numerous methods to accomplish this: chloramine-T (Greenwood, et al. Biochem J. 89: 114-123 (1963)); and Iodogen (Salacinski, et al. Anal. Biochem. 117: 136-146 (1981)). Tc and Re can be covalently bound through the sulfhydryl group of cysteine (Griffiths, et al. Cancer Res. 51: 4594-4602 (1991)). The problem with most of the techniques is that the body has efficient methods to break these covalent bonds, releasing the radioisotopes back into the circulatory system. Generally, these methods are acceptable for imaging purposes (^{99m}Tc), but not for therapeutic purposes.

172. Many peptide toxins have a generalized eukaryotic receptor binding domain; in these instances the toxin must be modified to prevent intoxication of cells not bearing the targeted receptor (e.g., to prevent intoxication of cells not bearing the “X” receptor but having a receptor for the unmodified toxin). Any such modifications must be made in a manner which preserves the cytotoxic functions of the molecule. Potentially useful toxins include, but are not limited to: cholera toxin, ricin, Shiga-like toxin (SLT-I, SLT-II, SLT-IIV), LT toxin, C3 toxin, Shiga toxin, pertussis toxin, tetanus toxin, Pseudomonas exotoxin, alorin, saporin, modeccin, and gelanin. Diphtheria toxin can be used to produce molecules useful as described herein. Diphtheria toxin, whose sequence is known, and hybrid molecules thereof, are described in detail in U.S. Pat. No. 4,675,382 to Murphy. The natural diphtheria toxin molecule secreted by Corynebacterium diphtheriae consists of several functional domains which can be characterized, starting at the amino terminal end of the molecule, as enzymatically-active Fragment A (amino acids Gly1-Arg193) and Fragment B (amino acids Ser194-Ser535), which includes a translocation domain and a generalized cell binding domain (amino acid residues 475 through 535). The process by which diphtheria toxin intoxicates sensitive eukaryotic cells involves at least the following steps: (i) the binding domain of diphtheria toxin binds to specific receptors on the surface of a sensitive cell; (ii) while bound to its receptor, the toxin molecule is internalized into an endocytic vesicle; (iii) either prior to internalization, or within the endocytic vesicle, the toxin molecule undergoes a proteolytic cleavage between fragments A and B; (iv) as the pH of the endocytic vesicle decreases to below 6, the toxin crosses the endosomal membrane, facilitating the delivery of Fragment A into the cytosol; (v) the catalytic activity of Fragment A (i.e., the nicotinamide adenine dinucleotide—dependent adenosine diphosphate (ADP) ribosylation of the eukaryotic protein synthesis factor termed “Elongation Factor 2”) causes the death of the intoxicated cell. A single molecule of Fragment A introduced into the cytosol is sufficient to inhibit the cell's protein synthesis machinery and kill the cell. The mechanism of cell killing by Pseudomonas exotoxin A, and possibly by certain other naturally-occurring toxins, is very similar.

173. A mixed toxin molecule is a molecule derived from two different polypeptide toxins. Generally, as discussed above in connection with diphtheria toxin, polypeptide toxins have, in addition to the domain responsible for generalized eukaryotic cell binding, an enzymatically active domain and a translocation domain. The binding and translocation domains are required for cell recognition and toxin entry respectively. Naturally-occurring proteins which are known to have a translocation domain include diphtheria toxin, Pseudomonas exotoxin A, and possibly other peptide toxins. The translocation domains of diphtheria toxin and Pseudomonas exotoxin A are well characterized (see, e.g., Hoch et al., Proc. Natl. Acad. Sci. USA 82:1692, 1985; Colombatti et al., J. Biol. Chem. 261:3030, 1986; and Deleers et al., FEBS Lett. 160:82, 1983), and the existence and location of such a domain in other molecules may be determined by methods such as those employed by Hwang et al. Cell 48:129, 1987; and Gray et al. Proc. Natl. Acad. Sci. USA 81:2645, 1984.

174. A useful mixed toxin hybrid molecule can be formed by fusing the enzymatically active A subunit of E. coli Shiga-like toxin (Calderwood et al., Proc. Natl. Acad. Sci. USA 84:4364, 1987) to the translocation domain (amino acid residues 202 through 460) of diphtheria toxin, and to a molecule targeting a particular cell type, as described in U.S. Pat. No. 5,906,820 to Bacha. The targeting portion of the three-part hybrid causes the molecule to attach specifically to the targeted cells, and the diphtheria toxin translocation portion acts to insert the enzymatically active A subunit of the Shiga-like toxin into the targeted cell. The enzymatically active portion of Shiga-like toxin, like diphtheria toxin, acts on the protein synthesis machinery of the cell to prevent protein synthesis, thus killing the cell.

175. The targeting molecule (for example, the antibody), and the cytotoxin can be linked in several ways. If the hybrid molecule is produced by expression of a fused gene, a peptide bond serves as the link between the cytotoxin and the antibody or antibody fragment. Alternatively, the toxin and the binding ligand can be produced separately and later coupled by means of a non-peptide covalent bond. For example, the covalent linkage may take the form of a disulfide bond. In this case, the DNA encoding the antibody can be engineered to contain an extra cysteine codon. The cysteine must be positioned so as to not interfere with the binding activity of the molecule. The toxin molecule must be derivatized with a sulfhydryl group reactive with the cysteine of the modified antibody. In the case of a peptide toxin this can be accomplished by inserting a cysteine codon into the DNA sequence encoding the toxin. Alternatively, a sulfhydryl group, either by itself or as part of a cysteine residue, can be introduced using solid phase polypeptide techniques. For example, the introduction of sulfhydryl groups into peptides is described by Hiskey (Peptides 3:137, 1981). The introduction of sulfhydryl groups into proteins is described in Maasen et al. (Eur. J. Biochem. 134:32, 1983). Once the correct sulfhydryl groups are present, the cytotoxin and antibody are purified, both sulfur groups are reduced; cytotoxin and ligand are mixed; (in a ratio of about 1:5 to 1:20) and disulfide bond formation is allowed to proceed to completion (generally 20 to 30 minutes) at room temperature. The mixture is then dialyzed against phosphate buffered saline or chromatographed in a resin such as Sephadex to remove unreacted ligand and toxin molecules.

176. Numerous types of cytotoxic compounds can be joined to proteins through the use of a reactive group on the cytotoxic compound or through the use of a cross-linking agent. A common reactive group that will form a stable covalent bond in vivo with an amine is isothiocyanate (Means, et al.). Chemical modifications of proteins (Holden-Day, San Francisco 1971) pp. 105-110). This group preferentially reacts with the ε-amine group of lysine. Maleimide is a commonly used reactive group to form a stable in vivo covalent bond with the sulfhydryl group on cysteine (Jo. Methods Enzymol 91: 580-609 (1983)). Monoclonal antibodies are incapable of forming covalent bonds with radiometal ions, but they can be attached to the antibody indirectly through the use of chelating agents that are covalently linked to the antibodies. Chelating agents can be attached through amines (Meares, et al., Anal. Biochem. 142:68-78 (1984)) and sulfhydral groups (Koyama Chem. Abstr. 120:217262t (1994)) of amino acid residues and also through carbohydrate groups (Rodwell, et al., Proc. Natl. Acad. Sci. 83:2632-2636 (1986); Quadri, et al., Nucl. Med. Biol. 20:559-570 (1993)). Since these chelating agents contain two types of functional groups, one to bind metal ions and the other to joining the chelate to the antibody, they are commonly referred as bifunctional chelating agents (Sundberg, et al., Nature 250:587-588 (1974)).

177. Crosslinking agents have two reactive functional groups and are classified as being homo or heterobifunctional. Examples of homobifunctional crosslinking agents include bismaleimidohexane (BMH) which is reactive with sulfhydryl groups (Chen, et al. J Biol Chem 266: 18237-18243 (1991) and ethylene glycolbis[succinimidylsucciate] EGS which is reactive with amino groups (Browning, et al., J. Immunol. 143: 1859-1867 (1989)). An example of a heterobifunctional crosslinker is m-malemidobenzoyl-N-hydroxysuccinimide ester (MBS) (Myers, et al. J. Immunol. Meth. 121: 129-142 (1989)). These methodologies are simple and are commonly employed.

13. Nucleic Acid Delivery

178. In the methods described above which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

179. As one example, vector delivery can be via a viral system, such as a retroviral vector system which can package a recombinant retroviral genome (see e.g., Pastan et al., Proc. Natl. Acad. Sci. USA 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used gene transfer methods.

180. As one example, if the encoding nucleic acid of the invention is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

181. Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

14. Delivery of the Compositions to Cells

182. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

183. Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

184. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

a) In vivo/ex vivo

185. As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

186. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

15. Expression Systems

187. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

188. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

189. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al. Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

190. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

191. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

192. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

193. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the constructs

b) Markers

194. The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

195. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media

196. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

16. Pharmaceutical Carriers/Delivery of Pharamceutical Products

197. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

198. The compositions may be administered orally, parenterally (e.g., intrvenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

199. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

200. The materials may be in solution; suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistc entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

201. The compositions, including antibodies, can be used therapeutically in combination with a pharmaceutically acceptable carrier.

202. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

203. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

204. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

205. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

206. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

207. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

208. Compositions for oral administration include powders or granules, suspensions or solutions in water or non-aqueous media, capsules, sachets, or tablets. Thickeners, flavorings, diluents, emulsifiers, dispersing aids or binders may be desirable.

209. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

210. Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

211. Following administration of a disclosed composition, such as an antibody, for treating, inhibiting, or preventing prostate cancer, the efficacy of the therapeutic antibody can be assessed in various ways well known to the skilled practitioner. For instance, one of ordinary skill in the art will understand that a composition, such as an antibody, disclosed herein is efficacious in treating or inhibiting prostate cancer in a subject by observing that the composition reduces a tumor mass or reduces the amount of PSA present in an assay, as disclosed herein. The compositions that inhibit prostate cancer as disclosed herein may be administered prophylactically to patients or subjects who are at risk for prostate cancer.

212. Other molecules that interact with the disclosed polypeptides, such as AR, Smad3, Smad4, TGF-B, Akt, IL-6, PTN to inhibit the disclosed interactions or modulate the disclosed AR activities, which do not have a specific pharmacuetical function, but which may be used for tracking changes within cellular chromosomes or for the delivery of diagnositc tools for example can be delivered in ways similar to those described for the pharmaceutical products.

17. Chips and Micro Arrays

213. Disclosed are chips where at least one address is the sequences or part of the sequences set forth in any of the nucleic acid sequences disclosed herein or molecular relationships disclosed herein. Also disclosed are chips where at least one address is the sequences or portion of sequences set forth in any of the peptide sequences disclosed herein or molecular relationships disclosed herein.

214. Also disclosed are chips where at least one address is a variant of the sequences or part of the sequences set forth in any of the nucleic acid sequences disclosed herein or molecular relationships disclosed herein. Also disclosed are chips where at least one address is a variant of the sequences or portion of sequences set forth in any of the peptide sequences disclosed herein or molecular relationships disclosed herein.

18. Computer Readable Mediums

215. It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

216. Also disclosed are any computer mediums containing the specific signal transduction relationships disclosed herein and/or their effects.

19. Compositions Identified by Screening with Disclosed Compositions/Combinatorial Chemistry

a) Combinatorial Chemistry

217. The disclosed compositions can be used as targets for any combinatorial technique to identify molecules or macromolecular molecules that interact with the disclosed compositions in a desired way. The nucleic acids, peptides, and related molecules, such as the disclosed polypeptides, such as AR, Smad3, Smad4, TGF-B, Akt, IL-6, PTEN or fragments thereof disclosed herein can be used as targets for the combinatorial approaches.

218. It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, the disclosed polypeptides, such as AR, Smad3, Smad4, TGF-B, Akt, IL-6, PTEN, are also disclosed. Thus, the products produced using the combinatorial or screening approaches that involve the disclosed compositions, such as, the disclosed polypeptides, such as AR, Smad3, Smad4, TGF-B, Akt, IL-6, PTEN, are also considered herein disclosed.

219. Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

220. There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

221. A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3 ′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

222. Another preferred method for combinatorial methods designed to isolate peptides is described in Cohen et al. (Cohen B. A., et al., Proc. Natl. Acad. Sci. USA 95(24): 14272-7 (1998)). This method utilizes and modifies two-hybrid technology. Yeast two-hybrid systems are useful for the detection and analysis of protein:protein interactions. The two-hybrid system; initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a AR, Smad3, Smad4, TGF-B, Akt, IL-6, and PTEN or fragment thereof, is attached to a DNA binding domain of a transcriptional activation protein, such as Gal 4. By performing the Two-hybrid technique on this type of system, molecules that bind the AR, Smad3, Smad4, TGF-B, Akt, IL-6, and PTEN or fragment thereof, can be identified.

223. Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

224. Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

225. Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. No. 5,698,685 and 5,506,337), sulfamides (U.S. Pat. No. 5,618,825), and benzodiazepines (U.S. Pat. No. 5,288,514).

226. As used herein combinatorial methods and libraries included traditional screening methods and libraries as well as methods and libraries used in interative processes.

b) Computer Assisted Drug Design

227. The disclosed compositions can be used as targets for any molecular modeling technique to identify either the structure of the disclosed compositions or to identify potential or actual molecules, such as small molecules, which interact in a desired way with the disclosed compositions.

228. It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, AR, Smad3, Smad4, TGF-B, Akt, IL-6, and PTEN or fragment thereof, are also disclosed. Thus, the products produced using the molecular modeling approaches that involve the disclosed compositions, such as, AR, Smad3, Smad4, TGF-B, Akt, IL-6, and PTEN or fragment thereof, are also considered herein disclosed.

229. Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

230. Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

231. A number of articles review computer modeling drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario Although these are primarily designed for application to drugs specific to particular proteins, they can be adapted to design of molecules specifically interacting with specific regions of DNA or RNA, once that region is identified.

232. Although described above with reference to design and generation of compounds which could alter binding, one could also screen libraries of known compounds, including natural products or synthetic chemicals, and biologically active materials, including proteins, for compounds which alter substrate binding or enzymatic activity.

20. Kits

233. Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for isolating molecules that effect the pathways disclosed herein, that comprises, for example, cells comprising AR and, for example, Smad3, Smad4, TGF-B, Akt, IL-6, and/or PTEN which can be used to assay compounds which modulate the effects of these proteins on AR.

D. Methods of Making the Compositions

234. The compositions disclosed herein and the compositions necessary to perform the disclosed methods can be made using any method known to those of skill in the art for that particular reagent or compound unless otherwise specifically noted.

1. Nucleic Acid Synthesis

235. For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

236. One method of producing the disclosed proteins, such as SEQ ID NO:23, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., N.Y. (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

237. For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K. et al., Biochemistry 33:6623-30 (1994)).

238. Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

239. Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

240. Disclosed are cells produced by the process of transforming the cell with any of the disclosed nucleic acids. Disclosed are cells produced by the process of transforming the cell with any of the non-naturally occurring disclosed nucleic acids.

241. Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

242. Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

243. Also disclosed are animals produced by the process of adding to the animal any of the cells disclosed herein.

244. Also disclosed are any compounds produced by identifying them using the disclosed methods and compositions and then synthesizing the identified compound or admixing the identified compound with a pharmaceutical carrier.

E. Methods of using the Compositions

1. Methods of Gene Modification and Gene Disruption

245. The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

246. One of the preferred characteristics of performing homologous recombination in mammalian cells is that the cells should be able to be cultured, because the desired recombination event occur at a low frequency.

247. Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

2. Method of Treating Cancer

248. The disclosed compositions can be used to treat any disease where uncontrolled cellular proliferation occurs such as cancers and the uncrontrolled proliferartion is related to AR activity, for example prostate cancer.

### F. EXAMPLES

249. The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Results

250. To dissect the divergent effects of IL-6 in prostate tumor growth, Qui, Y. et al., Nature 393, 83-85 (1998); Hobisch, A. et al., Cancer Res. 58, 4640-4645 (1998); Ritchie, C. K. et al., Endocrinology 138, 1145-1150 (1997), the potential influences of IL-6 on the transactivation of AR, the master regulator of the prostate tumor growth were studied. Chang, C. et al., Science 240, 324.326 (1988); Chang, C. et al., Crit. Rev. Eukaryotic Gene Expression 5, 97-125 (1995). Because IL-6 has two major signal cascades, mitogen-activated protein kinase (MAPK) and phosphatidylinositol 3-kinase (PI3K), Qui, Y. et al., Nature 393, 83-85 (1998); Qui, Y. et al., Proc. Natl. Acad. Sci. USA 95, 3644-3649 (1998), inhibitors were applied to specifically block one of these two IL-6 signal pathways. DU145 cells were transfected for 24 h with AR and MMTV-CAT reporter gene. After transfection, the cells were serum starved for 24 h. Next, 20 μM LY294002, P098059 or vehicle was added to serum-free medium 30 min prior to IL-6 treatment. After 30 min of treatment with IL-6, dihydrotestosterone (“DHT”) was added for another 24 h. The cells were then harvested and AR transactivation was measured by CAT activity. When none of the MAPK and PI3K pathways was blocked, IL-6 had a very limited effect on AR transactivation in the human prostate cancer DU145 cells (FIG. 1a). IL-6 could enhance AR transactivation via the MAPK pathway when the PI3K pathway was inhibited by LY294002 (FIG. 1a, lanes 6-8). Vlahos, C. J. et al., J. Biol. Chem. 269, 5241-5148 (1994). Alternatively, IL-6 could repress AR transactivation via the PI3K pathway when the MAPK pathway was inhibited by P098059 (FIG. 1a, lanes 10-12). Alessi, D. R. et al., J. Biol. Chem. 270, 27489-27494 (1995).

251. IL-6's effect on PI3K activity was tested. After serum starvation for 24 h, LNCaP, PC-3 or DU145 cells were treated with 50 μg/ml IL-6 for 30 minutes. The cells were then harvested. PI3K activity was measured by using phosphatidylinositol (PT) as a substrate. IL-6 was found to increase PI3K activity in prostate LNCaP, PC-3 and DU145 cells. It was demonstrated that this increase in PI3K activity by IL-6 in prostate LNCaP, PC-3 and DU145 cells could be repressed by LY294002 (FIG. 1b); The effect of Δp85, a dominant-negative form of a PI3K subunit, Hara, K. et al., Proc. Natl. Acad. Sci. USA 91, 7415-7419 (1994), in DU145 cells was examined. After 24 h transfection, the DU145 cells were treated with vehicle or LY294002 for 30 min prior to DHT treatment. The transactivation was measured by CAT activity. It was found that Δp85 could enhance AR transactivation in a dose-dependant manner in the presence of androgen (FIG. 1c). When the effect on AR transactivation by p110, the constitutively active form of PI3K, Hu, Q. et al., Science 268, 100-102 (1995), was tested in the same manner, a dose-dependent repression of AR transactivation by p110 was observed (FIG. 1d). Together, the results described in FIG. 1 provide strong evidence that IL-6 can repress AR transactivation via the PI3K pathway.

252. Focus then turned to the two major downstream effectors of PI3K: a serine/threonine kinase (Akt/PKB) and a ribosomal S6 kinase (p70S6k). Franke, T. F. et al., Cell 88,435-437 (1997). The DU145 cells were treated with LY294002 or rapamycin, an inhibitor of p70S6k, Seufferlei, T. et al., Cancer Res. 56, 3895-3897 (1996), for 30 min prior to DHT treatment. The transactivation activity was determined after 24 h transfection. The suppression of AR transactivation by PI3K was not influenced by the addition of rapamycin (FIG. 2a, lane 3 vs 6), indicating the PI3K-repressed AR transactivation might not function through the p70S6k pathway.

253. As shown in FIG. 2a, the data first showed the DHT-induced AR transactivation could be repressed by p110 (FIG. 2a, lane 2 vs 3). However, the addition of dAkt, a dominant-negative of Akt/PKB, Franke, T. F. et al., Cell 81,727-736 (1995), reversed the p110-repressed AR transactivation (FIG. 2a, lane 3 vs 4). The data also demonstrated that the LY294002-induced AR transactivation could be repressed by the addition of cAkt, a constitutive active form of Akt/PKB (FIG. 2a, lane 8 vs 9). Franke, T. F. et al., Cell 81,727-736 (1995). Furthermore, cAkt alone could also inhibit the DHT-induced AR transactivation FIG. 2a, lane 9 vs 10). FIG. 2b summarizes the suppression of AR transactivation through Akt/PKB by showing that cAkt could repress AR transactivation in a dose-dependant manner and dAkt could also induce AR transactivation in a dose-dependent manner. Together, the data from three different approaches described in FIG. 2 clearly demonstrated that Akt/PKB, but not p70S6k, is the downstream target to mediate PI3K-repressed AR transactivation.

254. Co-immunoprecipitation was used to demonstrate that Akt/PKB could interact directly with the AR. Cell lysates were immunoprecipitated (IP) with anti-Akt/PKB or normal IgG (N-IgG). The immunoprecipitated complexes were immunoblotted (IB) with AR antibody (NH27) or anti-Akt/PKB antibody, respectively. It was found that the anti-Akt/PKB antibody-precipitated complex from LNCaP whole cell extract contains the AR. This indicates that the AR can interact with Akt/PKB in vivo. Two purified E. coli expressed AR peptides that cover most of the N-terminal and DNA-binding domains (N-DBD, aa 36-643), or the DBD and ligand-binding domains (DBD-LBD, aa 553-918), were then used as the substrates for Akt/PKB. More specifically, 1 μg -DBD AR or 1 μg DBD-LBD AR purified from E. coli was treated for 1 h with Akt/PKB or PI3K. Phosphorylation of the AR was detected by separation on 12.5% SDS-PAGE and autoradiography. It was found that the N-DBD AR peptide could be phosphorylated by Akt/PKB. The DBD-LBD AR peptide can also be phosphorylated by Akt/PKB, but to a lesser extent.

255. Two putative Akt/PKB phosphorylation consenus sites (RXRXXS/T) have been identified in the AR at Ser210 (RAREAS) and Ser790 (RMRHLS). These two sites were individually mutated from serine to alanine. Expression vectors with wild type AR (wtAR) or either of the two mutant ARs (ARS210A and ARS790A) were transfected into DU145 and assayed for their ability to be phosphorylated by Akt/PKB. More specifically, wtAR, mtARS210A, or mtARS790A was transfected into DU145. After transfection, whole cell extract was immunoprecipitated with the anti-AR antibody, NH27. Half of the precipitated complex was treated with Akt/PKB, [gamma32p] ATP for 2 hand analyzed by SDS-PAGE. The remaining immunoprecipitates were subjected to western blot analysis to verify the equal expression levels of the wtAR and mtARAR constructs. As shown in FIG. 3a, the degree of phosphorylation of ARS210A and ARS790A by Akt/PKB, as compared to wtAR, was reduced significantly, suggesting these two sites could be targets for Akt/PKB phosphorylation.

256. A transient transfection assay was used to study the correlation between AR transactivation and the phosphorylation at Ser210 and Ser790. DU145 cells were transfected with plasmids encoding wtAR, mtARS210A or mtARS790S in conjunction with cAkt or dAkt for 24 h. The ligand treatment and transactivation was determined as previously described. The transient transfection assay revealed that the ARS210A transfected cells, but not those with wtAR, no longer possessed cAkt repressed AR transactivation (FIG. 3b, lane 2 vs 3 and lane 7 vs 8). The ability of dAkt to further promote AR transactivation was also reduced significantly in ARS210A cells (FIG. 3b, lane 2 vs 4 and lane 7 vs 9). Conversely, transfection with ARS790A has very little effect on Akt/PKB-mediated repression of AR transactivation (FIG. 3b, lanes 11-14). Together, results described above provide strong evidence that the AR is a downstream target for Akt/PKB. In addition, Ser210 of AR, but not Ser790, could be the essential phosphorylation target for the Akt/PKB-repressed AR transactivation.

257. Recent studies of steroid receptor mechanism suggested that steroid receptors might need coactivators for their proper or maximal transactivation. Onate, S. A. et al., Science 270, 1354-1357 (1995); Voegel, J. J. et al., EMBO J. 15, 3667-3675 (1996); Yeh, S. et al., Proc. Natl. Acad. Sci. USA 93, 5517-5521(1996); Yeh, S. et al., Proc. Natl. Acad. Sci. USA 95, 5527-5532 (1998); Fujimoto, N. et al., J. BioI. Chem. 274, 8316-8321 (1999); Miyamoto, H. et al., Proc. Natl. Acad. Sci. USA 95 11083-11088 (1998); Kang, H. Y. et al., J. BioI. Chem. 274, 8570-8576 (1999); Yeh, S. et al., Biochem. Biophys. Res. Commun. 248, 361-367 (1998). To study the molecular mechanism of PI3K-Akt/PKB repression of AR transactivation, a mammalian two-hybrid system was used to determine the potential effects of Akt/PKB on the interaction of AR and ARA70, an AR coactivator that can enhance AR transactivation. Yeh, S. et al., Proc. Natl. Acad. Sci. USA 93, 5517-5521(1996); Yeh, S. et al., Proc. Natl. Acad. Sci. USA 95, 5527-5532 (1998). The DU145 cells were transfected with 2.5 μg GAL4-ARA70 (GAL4DBD fused with ARA70) and 2.5 μg VP16-AR (VP16 fused with AR aa 36-918) followed by treatment with LY294002 or vehicle 30 min prior to DHT treatment. The interaction between AR and ARA70 was determined by CAT assay using pG5CAT as reporter. As shown in FIG. 4a, transient transfection of VP16-AR or GAL4-ARA70, without addition of DHT, showed negligible activity. However, the CAT activity could be induced by co-transfection of AR and ARA70 in the presence of 1 nM DHT (FIG. 4a, lane 1 vs 2). Interestingly, addition of dAkt or LY294002 further enhanced the interaction of AR and ARA70 (FIG. 4a, lane 2 vs 3 and lane 2 vs 6). In contrast, the addition of cAkt interaction between the AR and ARA70 (FIG. 4a, lane 2 vs 5 and lane 6 vs 8). Similar repression effects also occurred when we replaced ARA70 with other AR coactivators, such as ARA54 or TIF2. Kang, H. Y. et al., J. BioI. Chem. 274, 8570-8576 (1999); Voegel, J. J. et al., EMBO J. 15, 3667-3675 (1996). cAkt and dAkt, however, had very little effect on the interaction between GAL4 fused AR-LBD653-918 and VP16-ARA 70 (VP16 fused ARA70), VP16-ARA54, or VP16-TIF2. The MMTV-CAT reporter assay further confirmed that the enhanced AR transactivation by various AR coactivators, such as ARA70, ARA54, TIF2 or SRC-1, Onate, S. A. et al., Science 270, 1354-1357 (1995), could be further promoted in the presence of LY294002 or Δp85 (FIG. 4c). Together, these data indicate the suppression of AR transactivation by PI3K-Akt/PBK can involve the inhibition of interaction between AR and ARAs.

258. The findings that only the interaction between VP16-AR₃₆-918 and GAL4-ARA70, but not VP16-ARA70 and GAL4-ARLBD₆₅₃₋₉₁₈ can be repressed by cAkt suggests Akt/PKB may repress the interaction of AR and ARAs through the AR N-terminal domain. This is consistent with the above conclusions that N-terminal Ser210 is the essential phosphorylation site for Akt/PKB. Together, the discovery that phosphorylation of the AR N-terminal by Akt/PKB can repress AR transactivation, through inhibiting the interaction of AR and ARAs, represents a molecular mechanism to explain how phosphorylation can repress AR transactivation.

259. The data indicate a signaling pathway by which IL-6 suppresses AR transactivation: IL-6 activation of PI3K activates Akt/PKB, and Akt/PKB phosphorytates the AR on Ser210, inhibiting its transactivation. DHT's effect on IL-6 protein expression in LNCaP cells was tested by treating the cells with 0, 1, 10, or 50 nM DHT for 24 h, followed by 160 nM phorbol-12-myristate-13-acetate (PMA) treatment for an additional 24 h. The IL-6 protein level in the supernatants was then evaluated by BLISA. As shown in FIG. 5a, IL-6 protein expression was reduced by DHT in a dose-dependant manner and addition of hydroxyflutamide (HF), an antiandrogen, could reverse this induction, suggesting AR plays an essential role for this repression.

260. Next, the effect of DHT on the IL-6-induced PI3K activity was tested. LNCaP cells were serum starved for 24 h and then were treated with DHT and IL-6 for 30 minutes. PI3K activity was measured using PI as a substrate. As shown in FIG. 5b, addition of DHT could repress PI3K activity significantly in the presence of exogenous IL-6, indicating that DHT's dampening effect on PI3K activity was not only a result of repressing the expression of IL-6. The results in FIG. 5 demonstrate the existence of bidirectional regulation between IL-6 and androgen-AR indicating that androgen-AR could repress the IL-6/PI3K signaling pathway through a feed-back control mechanism. This new signal cascade is summarized in FIG. 6.

b) Materials and Methods

(1) Materials.

261. DHT was obtained from Sigma. LY294002, PD98059, PMA, and IL-6 were purchased from Calbiochem. Antibodies to Akt and PI3K subunit p85 were from New England Biolabs and Upstate Biotechnology, respectively. NH27 Anti-AR polyclonal antibody was produced as previous described. Chang, C. et al., Crit. Rev. Eukaryotic Gene Expression 5, 97-125 (1995).

(2) Cell Culture and Transfections.

262. The DU145 and PC-3 cells were maintained in Dulbecco's Minimum Essential Medium (DMEM) containing penicillin (25 μg/m1), streptomycin (25 μg/ml), and 5% fetal calf serum (FCS). The LNCaP cells were maintained in RPMI-1640 with 10% FCS. Transfections were performed using the calcium phosphate precipitation method, as previously described. Yeh, S. et al., Proc. Natl. Acad. Sci. USA 93, 5517-5521(1996).

(3) Site-directed Mutagenesis of AR.

263. pSG5-wtAR was used as the DNA mutagenesis template to anneal with mutagenic primers: 5′-AGGGAGGCCGCGGGGGCT-3′ and 5′-AGGCACCTCTCTCAAGAGTT-3′. The mutant strand was synthesized with T4 DNA polymerase and T4 DNA ligase by using the Gene Editor kit (Promega) and was used to transform BMH71-18 muS cells. The plasmid DNAs were isolated from the selection plates and then transformed into JM109 cells. The mutant plasmids were then confirmed by DNA sequencing.

(4) Immunoprecipitation, Western Blotting, and in vitro AR Phosphorylation.

264. The immunoprecipitation and western blotting were performed as previously described. Qui, Y. et al., Nature 393, 83-85 (1998). AR phosphorylation was assayed as described. Yeh, S. et al., Proc. Natl. Acad. Sci. USA. Briefly, PI3K LNCaP cells were stimulated with IL-6 (50 μg/ml) for 30 minutes. Next, PI3K or Akt was immunoprecipitated from the LNCaP cells. The immunoprecipitated PI3K or Akt was incubated with 1 μg purified AR peptide in HEPES buffer containing 20 mM HEPES, pH 7.4, 10 mM MgCl², 10 mM DTT, 2 μM ATP, and 10 μCi [y-³²P]ATP, at room temperature for 1 h. Reactions were stopped by adding an equal volume of 2×SDS loading buffer. SDS-PAGE and autoradiography were then performed.

(5) PI3K Activity Assay.

265. PI3K activity was determined as previously described. Vlahos, C. J. et al, J. Biol. Chem. 269, 5241-5148 (1994).

### 2. Example 2

Akt Suppresses Androgen-induced Apoptosis by Phosphorylating and Inhibiting Androgen Receptor

266. References cited in this example not specifically designated can be found in reference list A. Thus, a number designation of the reference refers to that number reference in Reference A.

a) Results

(1) Akt Phosphorylates AR in vitro.

267. Because the region surrounding Ser210 (RAREAS) and Ser790 (RMRHLS) in AR conforms to a consensus sequence (RXRXXS/T) of the Akt phosphorylation site, these two Ser sites were mutated to alanine, which cannot be phosphorylated. Expression vectors with wild-type AR (wtAR) or either one of the two mutant ARs (mtAR S210A and mtAR S790A) were then transfected into prostate cancer DU145 cells without endogenous AR and assayed for their ability to be phosphorylated by Akt in vitro. As shown in FIG. 7A, the degree of Akt phosphorylation of mtARS210A and mtARS790A, as compared to wtAR, was reduced significantly, indicating these two sites could be targets for Akt phosphorylation.

268. Co-immunoprecipitation was conducted to demonstrate that Akt can interact with the AR in vivo. As shown in FIG. 7B, the anti-Akt antibody-precipitated complex from LNCaP whole cell extract contained the AR indicating that the AR could interact with Akt in vivo. Two purified E. coli. expressed AR peptides that cover most of the N-terminal and DNA-binding domains (N-DBD, aa 36-643) (Yeh et al. (1999) Proc Natl Acad Sci USA 96, 545863), or the DBD and ligand-binding domains (DBD-LBD, aa 553-918) (Yeh et al.), were then used as the substrates for Akt. As shown in FIG. 7C, Akt phosphorylated the N-DBD AR peptide. The DBD-LBD AR peptide could also be phosphorylated by Akt. In contrast, PI(3)K failed to phosphorylate either N-DBD or DBD-LBD, indicating that phosphorylation of AR by Akt is specific.

(2) Akt Phosphorylates AR in vivo and Inhibits AR Transactivation.

269. Activation of Akt by IGF-1 in COS-1 cells can be blocked by LY294002, a specific PI(3)K blocker was demonstrated (FIG. 8A). FIG. 8B further showed that IGF-1 strongly induced AR phosphorylation and its effect was blocked by LY294002, indicating IGF-1 can phosphorylate AR via the PI(3)K/Akt pathway in vivo. Furthermore, FIG. 8C showed that the constitutively active Akt (cAkt) (Franke et al. (1995) Cell 81, 727-36), but not the dominant-negative Akt (dAkt) (Franke et al.), phosphorylated wt AR, but not mutant ARs (mtAR S210A or mtAR S790A), which is in agreement with these in vitro results (FIG. 7A). cAkt and dAkt were then applied to test if phosphorylation of AR by Akt may result in the modulation of AR transactivation. As shown in FIG. 8D, cAkt could repress wtAR transactivation in a dose-dependent manner and dAkt could induce wtAR transactivation in a dose-dependent manner in DU145 cells. The finding that dAkt enhanced the AR transactivation (FIG. 8D) indicates that the endogenous Akt activity might contribute to the suppression of the AR transactivation. Similar results were also observed in PC-3 and LNCaP cells. Modulation of AR transactivation by Akt was further confirmed by using two AR mutants, mtAR S210A and mtAR S790A, in transient transfection assays. As shown in FIG. 8D, while cAkt could still repress wtAR-mediated transactivation, cAkt has less ability to repress mtAR S210A-mediated transactivation (FIG. 8E). The ability of dAkt to further promote AR transactivation was also reduced significantly in mtAR S210A (FIG. 8E). Conversely, transfection with mtAR S790A has very little effect on Akt-mediated repression of AR transactivation (FIG. 8E). Together, data from FIG. 2 indicate that AR is a new substrate for Akt and Ser210, but not Ser790 in AR, could be the essential phosphorylation site to mediate the Akt-repressed AR transactivation. The data (FIG. 8E) show that cAkt can still suppress the mtAR S210A transactivation, and thus also disclosed are mutated sites other than S210 and S790 that contribute to the modulation of AR activity.

(3) Akt is a Downstream Effector to Mimic PI(3)K Effect on Suppression of AR Transactivation.

270. The finding that Akt could phosphorylate and inhibit AR transactivation was further extended to the Akt upstream activator, PI(3)K. In DU145 cells the effect of Δp85, a dominant-negative form of PI(3)K was examined, and found that, in the presence of androgen, Δp85 can enhance AR transactivation in a dose-dependant manner (FIG. 9A). LY294002, an inhibitor of PI(3)K, also showed enhancement of AR transactivation. Taken together, theses results indicate that both LY294002 and Δp85 may be able to interrupt the endogenous PI(3)K activity which negatively regulates AR transactivation. The data also showed that AR transactivation can be repressed by p110* (Hu et al. (1995) Science 268, 100-2), the constitutively active form of PI(3)K in a dose-dependant manner (FIG. 9B).

271. That the suppression of AR transactivation by p110* was not influenced by the addition of rapamycin, an inhibitor of a ribosomal S6 kinase (p70S6K) was demonstrated (FIG. 9C). This indicated that the PI(3)K-repressed AR transactivation does not go through the p70S6K pathway. FIG. 9C showed that the dihydrotestosterone (DHT)-induced AR transactivation could be repressed by p110* and the addition of dAkt reversed this p110*-repressed AR transactivation (FIG. 9C). Furthermore, the LY294002-induced AR transactivation could be repressed by the addition of cAkt (FIG. 9C). Together, the data from different approaches described in FIG. 9 demonstrate that the PI(3)K/Akt, but not PI(3)K/p70S6K signaling pathway, can modulate the AR transactivation. As LY294002 has more potent effect on AR transactivation than Δp85 and dAkt, it is also possible that signal pathways other than PI3K/Akt can involve in LY294002 action.

(4) Suppression of the Interaction Between AR and AR Coregulators by PI(3)K/Akt Pathway.

Recent studies suggest that steroid receptors might require the presence of coregulators for their proper or maximal transactivation (McKenna et al. (1999) Endocr Rev 20, 321-44; Yeh et al. (1996) Proc Natl Acad Sci USA 93, 5517-21; Yeh et al. (1998) Biochem Biophys Res Commun 248, 361-7). To study the molecular mechanism of PI(3)K-Akt repression of AR transactivation, a mammalian two-hybrid systemwas used to determine the potential effects of Akt on the interaction of AR and ARA70, an AR coregulator that can enhance AR transactivation. GAL4DBD fused to ARA70 aa 176-401 (GAL4-ARA70), and VP16 fused to the AR aa 36-918 (VP16-AR) were transfected into DU145 cells in the presence or absence of cAkt, dAkt, and LY294002. As shown in FIG. 10A, transient transfection of VP16-AR and GALA-ARA70, without addition of DHT, showed negligible activity. However, the chloramphenicol acetyltransferase (CAT) activity could be induced by co-transfection of AR and ARA70 in the presence of 1 nM DHT. Addition of dAkt or LY294002 further enhanced the interaction between the AR and ARA70.

272. In contrast, addition of cAkt repressed the AR and ARA70 interaction as well as the LY294002 enhancement of the interaction between the AR and ARA70 (FIG. 10A). Similar repression effects also occurred when ARA70 was replaced with other AR coregulators, such as ARA54 (Kang et al. (1999) J Biol Chem 274, 8570-6) or TIF-2 (McKenna et al. (1999) Endocr Rev 20, 321-44). cAkt and dAkt, however, had very little effect on the interaction between GAL4 fused AR-LBD (aa 653-918) and VP16 fused ARA70 (VP16-ARA70), VP16-ARA54, or VP16-TIF2. Using MMTV-CAT reporter, it was confirmed that the induced-AR transactivation by various AR coregulators, such as ARA70, ARA54, TIF2, or SRC-1 (McKenna et al.), could be further enhanced in the presence of LY294002 or Δp85 (FIG. 10B). Together, these data indicate the suppression of AR transactivation by PI(3)K-Akt can involve the inhibition of AR and ARAs interaction.

273. The findings that only the interaction between VP16-AR (aa 36-918) and GAL4-ARA70, but not VP16-ARA70 with GAL4-ARLBD (aa 653-918), can be repressed by cAkt indicate Akt can repress the interaction of AR and ARAs through the AR N-terminal domain. This is consistent with the above conclusion that phosphorylation of the N-terminal Ser210mediates Akt repression of AR transactivation.

274. While most data indicate that androgen/AR can be critically involved in the proliferation of prostate cancer (Prehn, R. T. (1999) Cancer Res 59, 4161-4), the opposite roles of androgen/AR in the inhibition of cell growth and promotion of apoptosis are also well documented (Heisler, et al. (1997) Mol Cell Endocrinol 126, 59-73; Yuan et al. (1993) Cancer Res 53, 1304-11). To correlate PI(3)K/Akt suppression of AR transactivation to androgen-induced apoptosis, it was demonstrated that addition of androgen could induce cell apoptosis in prostate cancer cells PC-3(AR)2, PC-3(AR)6, and thymoma SAR-91 cells that were stably transfected with AR (FIG. 11B). Compared to the parent cell line PC-3(M), Western blot analyses indicated PC-3(AR)2, and PC-3(AR)6 cells express similar amounts of AR (FIG. 11A). Addition of hydroxyflutamide (HF), an antiandrogen, showed inhibition of androgen-induced apoptosis, indicating AR plays an essential role in the apoptosis (FIG. 11B). Furthermore, it was found that IGF-1 could repress androgen-induced apoptosis, and this repression could be reversed by the addition of LY294002 (FIG. 11B). We also determined that addition of androgen showed no apoptotic effect in S7MC, the thymoma parent cell line (FIG. 11B), or PC-3(M). Together, FIG. 11 demonstrate that the PI(3)K/Akt pathway can modulate androgen-induced apoptosis and AR can function as a proapoptotic factor in prostate cancer or thymoma cells.

(5) Suppression of A/AR-induced Apoptosis and p21 Expression by PI(3)K/Akt Pathway.

275. As shown in FIG. 12A, 10 nM DHT could induce p21 promoter activity and cAkt could repress p21 expression in a dose-dependent manner in PC-3 cells. In contrast, dAkt enhanced p21 expression in a dose-dependent manner (FIG. 12A). This Akt-regulated p21 protein expression also correlated well with the androgen-induced apoptosis that was suppressed by cAkt in PC-3(AR)6 cells (FIG. 12B). Similar correlations between the PI(3)K/Akt pathway, androgen-induced apoptosis, and p21 expression also occurred in LNCaP cells, which express functional AR. FIG. 12C showed that LNCaP cells stably transfected with dAkt have a 50% reduction in Akt activity that resulted in the considerably enhancement of p21 expression in response to androgen from 2.5 fold to 14.5 fold.

276. The expression of p21, again correlated very well with LNCaP cell apoptosis that is induced by 10 nM DHT and 1 nM 12-0-tetradecanoyl phorbol-13-acetate (TPA), the activator of the PKC (FIG. 12D). Addition of HF could then repress this DHT/TPA-mediated apoptosis (FIG. 12D). In contrast, DHT or TPA, per se, had only marginal effects on apoptosis, indicating that DHT and TPA cooperatively induced LNCaP cell apoptosis (FIG. 12D). IGF-1 activation of PI(3)K/Akt pathway partially repressed DHT/TPA-induced apoptosis in LNCaP parent cells, and LY294002 could reverse this IGF-1 suppression (FIG. 12E). In contrast, IGF-1 showed only marginal suppressive effects on DHT/TPA-induced apoptosis in LNCaP cells stably transfected with dAkt. Together, FIG. 12 demonstrates that the PI(3)K/Akt pathway is able to suppress the DHT/TPA-induced apoptosis in LNCaP cells which has positive correlation to the p21 expression.

b) Materials and methods

(1) Materials.

DHT was obtained from Sigma. LY294002, TPA, and IGF-1 were purchased from Calbiochem. Antibodies to Akt, PI(3)K subunit p85, and p21 were from New England Biolabs, Upstate Biotechnology, and Santa Cruz, respectively. The anti-AR polyclonal antibody, NH27, was produced as previously described (McKenna et al. (1999) Endocr Rev 20, 321-44; Yeh et al. (1996) Proc Natl Acad Sci USA 93, 5517-21; Yeh et al. (1998) Biochem Biophys Res Commun 248,361-7). Δp85 was kindly provided by Dr. M. Kasuga (Sakaue et al. (1995) J Biol Chem 12, 11304-9) and p110* was from Dr. L. Williams (Hu et al. (1995) Science 268, 100-2). pCDNA3, cAkt (a constituetively active Akt with a deletion at aa 4-129 replaced with a consensus myristylation domain) and pCDNA3 dAkt (a kinase deficient mutant, K179A) were from Dr. R. Freeman (Crowder, et al. (1998) J Neurosci 18, 2933-43). PC-3(AR)2 and pC-3(AR)6 were from Dr. T. J. Brown (Heisler et al. (1997) Mol Cell Endocrinol 126, 59-73) and thymocytes S7MC and SAR-91 were from R. L. Miesfeld (Chapman et al. (1996) Mol Endocrinol 10, 967-78).

(2) Cell Culture and Transfections.

277. The DU145 and PC-3 cells were maintained in Dulbecco's Minimum Essential Medium (DMEM) containing penicillin (25 U/ml), streptomycin (25 μg/ml), and 5% fetal calf serum (FCS). The LNCaP cells were maintained in RPMI-1640-10% FCS. Transfections were performed using the calcium phosphate precipitation method in PC-3 and DU145, as previously described. Yeh, S., Lin, H. K., Kang, H. Y., Thin, T. H., Lin, M. F. & Chang, C. (1999) Proc Natl Acad Sci U S A 96, 5458-63. LNCaP cells were transfected using SuperFect™ according to standard procedures (Qiagen).

(3) Site-directed Mutagenesis of AR.

278. pSG5-wtAR was used as the DNA mutagenesis template to anneal with mutagenic primers: 5′-AGGGAGGCCGCGGGGGCT-3′ and 5′-AGGCACCTCTCTCAAGAGTTT-3′. The mutant strand was synthesized with T4 DNA polyrerase and T4 DNA ligase using the Gene Editor kit (Promega) and then used to transform BMH71-18 muS cells. The plasmid DNAs were isolated from the selection plates and then transformed into JM109 cells. The mutant plasmids were then confirmed by DNA sequencing.

(4) Immunoprecipitation, Western Blotting, and in vitro AR Phosphorylation.

279. The immunoprecipitation, western blotting, and AR phosphorylation were performed as previously described (Qiu et al. (1998) Nature 393, 83-5). Briefly, immunoprecipitated PI(3)K or Akt from LNCaP cells stimulated with IGF-1 (50 μg/ml) for 30 min, were incubated with 1 μg purified AR peptide in HEPES buffer (20 mM HEPES/pH 7.4, 10 mM MgCl₂, 10 mM DTT, and 2 μM ATP) and 10 μCi [γ-³²P]ATP, at room temperature for 1 h. Reactions were stopped by adding an equal volume of 2×SDS loading buffer and subjected to SDS-PAGE, followed by autoradiography. To confirm that the Akt and PI(3)K used in this experiment are active, the histone 2B (H2B) and phosphatidylyinositol (PI) were utilized as a substrate for Akt and PI(3)K, respectively.

(5) LNCaP Stable Transfections.

280. The LNCaP cells were transfected with pcDNA3 or pcDNA3 dAkt for 24 h. The cells were selected using 300 μg/ml neomycin (GibcoBRL). Individual single colonies were picked and confirmed by western blot analysis.

(6) Apoptosis Assay.

281. The TUNEL assay was performed to measure the cell apoptosis according to the standard procedures (Oncogene Research Products). At least 200 cells were scored for each sample and the data are means±s.d. from three independent experiments.

(7) In vivo AR Phosphorylation.

282. For labeling experiments, COS-1 cells were cultured in DMEM-10% FCS and transfected with PSG5-AR using SuperFect™ according to standard procedures (Qiagen) for 24 h, the medium was changed to phospho-free DMEM-10% FCS containing 200 μCi/ml ortho-[³²P] (New England Nuclear) for 4 h. During the [³²P] labeling, cells were pretreated with ethanol or LY294002 for 30 min, followed by IGF-1 treatment for 2 h Cells were lysed by RIPA buffer, and the total cell lysates were incubated with NH27. The AR immunocomplex was subjected to SDS-PAGE followed by autoradiography.

### 3. Example 3

### From TGF-β Signaling to Androgen Action: Identification of Smad3 as an Androgen Receptor Coregulator in Prostate Cancer Cells

283. References cited in this example not specifically designated can be found in reference list B. Thus, a number designation of the reference refers to that number reference in Reference B.

a) Results

(1) Enhancement of AR-mediated Transactivation by TGF-β in Different Prostate Cancer Cells.

284. To study the potential correlation between androgen and TGF-β in prostate cancer cells, the TGF-β responsive prostate cancer DU145 and PC-3 cells were chosen to examine the effect of TGF-β on androgen-induced mouse many tumor virus (MMTV) promoter activity. Activation of MMTV-CAT activity was achieved by transient transfection of AR in the presence of 10⁻⁸ M DHT (FIG. 13A, lane 1-3) and this AR-mediated transactivation was enhanced by the addition of TGF-β in DU145 cells (FIG. 1A, lane 3 vs 5). Furthermore, this induction was partially blocked by adding TGF-β specific neutralizing antibody (FIG. 13A, lane 5 vs. 6). Similar results were obtained with PC-3 cells, where AR-mediated transactivation was enhanced by TGF-β (FIG. 13B, lane 2 Vs 3-5) and suppressed by the TGF-β specific neutralizing antibody (FIG. 13B, lane 6 vs. 7-10), both in a dose-dependent manner (FIG. 13B). Since western blot analysis indicated PC-3 cells stably transfected with AR, PC-3(AR)2, express similar amounts of AR as compared to LNCaP cells and the increased AR-mediated transactivation by TGF-β did not change the expression level of AR (data not shown), the effect of TGF-β receptors in PC-3(AR)2 was examined. As shown in FIG. 13C, in the presence or absence of androgen, TβRI or TβRII receptor alone have marginal effect on AR mediated transactivation. However, coexpression of TβRI and TβRII receptor, or constitutively active TGF-β type I receptor (TβRI-T204D) could further enhance AR transactivation in the presence of DHT. Taken together, these data indicate TGF-β can cross-talk with the androgen/R pathway without altering the expression of AR.

(2) Association Between AR and Smad3 in vitro and in vivo.

285. Interaction between AR and Smad3, the mediator of TGF-β signaling, is shown herein. The mammalian two-hybrid assay was applied in SW480.7 cells that lack Smad4, but still express Smad1 and Smad3 (Sato et al. (1997) J Biol Chem 272, 17485-94).The results show that DHT, at concentrations greater than 1 nM, promotes the interaction between Smad3 and AR (FIG. 14A, lane 7), indicating that Smad3 is sufficient to interact with AR. To further explore the mechanism underlying this association between AR and Smad3, prostate DU145 cells were treated with TGF-β to determine whether TGF-β was involved. As shown in FIG. 14B, transient transfection of either Gal4-Smad3 or VP16-AR alone showed negligible activity (lane 2-5). The CAT activity was induced when VP16-AR was co-expressed with Gal4-Smad3 in the presence of 10 nM DHT (lane 7, hatched bar). Upon TGF-β stimulation the reporter gene activity was further induced (lane 7, solid bar), however, TGF-β cannot exert this effect in the absence of DHT (lane 6). These results indicate that the association between AR and Smad3 is an androgen-dependent process and TGF-β can further enhance this interaction.

286. To further demonstrate the interaction between AR and Smad3, N-terminal Flag-tagged, full-length Smad3 was expressed in PC-3 cells alone or in the presence of wtAR. Cell extracts were prepared and immunoprecipitations were performed using anti-Flag antibodies followed by Western blotting utilizing anti-AR antibodies. In the presence of Flag-Smad3, AR was co-immunoprecipitated with Smad3 both in the presence or absence of 10 nM DHT (FIG. 15A). Next, An In vivo co-immunoprecipitation assay was applied to demonstrate that the endogenous Smad3 is capable of interacting with AR. As shown in FIG. 15B, AR was detected in the Smad3 immunocomplex in the absence or presence of androgen in PC-3(AR)2 but not in PC-3 cells. A similar result was also obtained when we replaced PC-3(AR)2 with LNCaP cells.

287. To determine which individual domain of AR can interact with Smad3, GST-Smad3 fusion proteins incubated with various AR deletion mutants (shown in FIG. 15C) were used in pull-down experiments (FIG. 15D). The full-length wtAR could interact with Smad3 both in the presence and absence of 10 nM DHT. While DBD-LBD AR peptides can interact with Smad3, it was found that both DBD AR and LBD AR peptides interact with Smad3 but N-terminal AR peptide failed to interact with Smad3. Furthermore, two AR mutants (mtAR R614H with mutation at the second zinc finger of the DBD and mtAR E708K with mutation at the LBD) were still able to interact with Smad3. These results indicate that AR can contain two independent binding sites located in both DBD and LBD domains to interact with Smad3.

(3) Roles of Smad3 in AR-mediated Transactivation.

288. Smad3 can enhance androgen-induced AR transactivation in SW480.7 cells which are unresponsive to the inhibitory effects of TGF-β. As shown in FIG. 16A, Smad3 increased the ligand-dependent transactivation of AR, indicating that Smad3 was able to function as a positive AR coregulator to enhance AR transactivation. Similarly, the enhanced transactivation function of AR by Smad3 was observed in DU145 cells (FIG. 16B). A C-terminal deletion of 39 amino acids resulted in the loss of the Smad3 enhanced effect of the MMTV-CAT reporter gene in DU145 cells. As previous reports showed that a MH2 region in the C-terminal region of Smad3 is essential for homo-oligomerization and hetero-oligomerization (Massague, J. (1996) Cell 85, 947-50), it is possible that this region is also important for Smad3 to interact with AR and exert its function as an active coregulator for AR.

(4) Androgen-response Element (ARE) is Important for TGF-β/Smad3-enhanced AR Transactivation.

289. To test whether the ARE is important for TGF-β and Smad3 to enhance AR-mediated transactivation, DU145 cells were transiently transfected with MMTV and PSA, two of the AR target natural promoters, and one synthetic promoter, tyrosine aminotransferase (TAT)₂, which contains only two copies of a synthetic ARE. As shown in FIG. 17A and 17B, increasing AR led to a higher degree of transactivation in a DHT-dependent manner and TGF-β and Smad3 were able to further enhance both the natural and synthetic ARE promoters.

290. To rule out any indirect effects on the basal activity of the MMTV-ARE CAT reporter, the ARE DNA fragment from our reporter (MMTV-ΔARE-CAT) was removed. The results showed that TGF-β and Smad3 could not induce any activity. Taken together, these results indicate that the ARE is essential for TGF-β/Smad3 to exert their influence on AR transactivation.

(5) Effect of Smad3 on the Transactivation of the Progesterone Receptor, Vitamin D Receptor (VDR), Estrogen Receptor, wtAR, and mtAR.

Several identified coregulators, such as SRC-1 (Onate et al. (1995) Science 270, 1354-7), CBP/p300 (Kamei et al. (1996) Cell 85,403-14) and GRIP1/TIF2 (Hong et al. (1996) Proc Natl Acad Sci USA 93, 4948-52; Voegel et al. (1996) Embo J 15, 3667-75), enhance the transactivation of most steroid receptors. It is therefore important to investigate whether Smad3 can function as a general coregulator for other steroid receptors through their cognate ligands and response elements in prostate cells. Among all the classic steroid receptors tested, Smad3 could significantly enhance the transactivation of AR, the progesterone receptor, and VDR (FIG. 18A). This data is also in agreement with the previous report showing Smad3 can interact with VDR and enhance VDR target genes (Yanagisawa et al. (1999) Science 283, 1317-21). Since androgen signal pathway is opposite of the Vitamin. D signal pathway in the modulation of prostate cell growth, Identification of Smad3 as an AR positive coregulator can provide an explanation for TGF-β signals in androgen-mediated prostate cancer cell growth.

291. It is thought that prostate cancer progresses from an androgen-dependent to an androgen-independent stage via mutations in AR which change the specificity and sensitivity of AR to antiandrogens, such as HF (Miyamoto et al. (1998) Proc Natl Acad Sci USA 95, 7379-84). Results from DU145 cells show that wtAR responded well to DHT at 10 nM, and Smad3 enhanced this transactivation to another 3-4 fold (FIG. 18B). On the other hand, wtAR was only able to respond marginally to 1 μM HF and 10 nM E2, but Smad3 could further promote the wtAR transactivation in the presence of 1 μM HF and 10 nM E2. These findings were extended to the AR mutant; mtARt877a, which is found in many prostate tumors and LNCaP cells (Gaddipati et al. (1994) Cancer Res 54, 286-14). Previous reports showed that LNCaP mtARt877a could be stimulated by E2, progesterone, and flutamide (Gaddipati et al. (1994) Cancer Res 54, 286-14). In comparison, this data showed mtARt877a responded much better to HF and E2 than wtAR (FIG. 18C). Furthermore, Smad3 could promote this E2- or HF-mediated androgenic activity on mtARt877a. Compared to the previously identified coregulator, ARA70, Smad3 showed a relatively stronger enhancement effect on the AR transactivation. Together, these results indicate that SMAD 3 enhances the DHT-, E2-, or HF-mediated transactivation in LNCaP AR cells.

(6) AR-induced PSA Expression is Enhanced by Smad3.

292. A previous study reported that plasma TGF-β was significantly elevated in patients with clinically evident prostate metastases and correlated with PSA levels (Ivanovic et al. (1995) Nat Med 1, 282-4; Adler et al. (1999) J Urol 161, 182-7. Therefore, it is important to investigate the effect of Smad3 on androgen-induced PSA expression in order to understand the mechanism of prostate carcinoma progression. As shown in FIG. 17, increasing AR induced PSA reporter gene activity in a DHT-dependent manner and TGF-β or Smad3 were able to further enhance PSA promoter activity. Using Northern blot analysis, the data shows that endogenous PSA expression in LNCaP cells can also be induced by DHT. Addition of Smad3 can further enhance PSA expression in the presence of androgen (FIG. 19A, lane 2 vs. 3). As a control, the data also demonstrated that addition of Smad3 failed to induce PSA expression in the absence of androgen (FIG. 19A, lane 1 vs. 4). Furthermore, this Smad3-enhanced PSA induction can be partially repressed by HF, indicating that Smad3 can play positive roles in enhancing PSA expression via cooperation with AR in the presence of androgen.

b) Materials and Methods

(1) Chemicals and Plasmids.

293. DHT, dexamethasone, progesterone, and E2 were obtained from Sigma, and hydroxyflutamide (HF) from Schering, USA. pSG5-wild type AR (wtAR), pCMV-AR, and pCMV-mtARt877a (mutant AR derived from the prostate cancers, codon 877 mutation threonine to alanine) were used in our previous report (4). Expression plasmids for glutathione S-transferase (GST)-Smad3 and full-length cDNAs of human Smad3, were kindly provided by Rik Derynck (28). TβRI, TβRII receptors and constitutively active TGF-β type I receptor (TβRI-T204D) expression vectors were provided by Dr. Jeffery L. Wrana (Wrana et al. (1994) Nature 370, 341-7).

(2) Cell Culture and Transfections.

294. Human prostate cancer DU145 cells and PC-3 cells were maintained in Dulbecco's Minimum Essential Medium containing penicillin (25 U/ml), streptomycin (25 μg/ml), and 5% fetal calf serum Transfections were performed using the calcium phosphate precipitation method and cells were harvested after 24 hours for the chloramphenicol acetyltransferase (CAT) assay, as described previously (Fujimoto et al. (1999) J Biol Chem 274, 8316-21). The CAT activity was visualized and quantitated by STORM 840 (Molecular Dynamics). At least three independent experiments were carried out in each case. The SW480.7 cells and PC3 (AR)2 cells are the gifts from Dr. Eric J. Stanbridge and Dr. T. J. Brown.

(3) GST Pull-down Assay.

295. Fusion proteins of GST-Smad3 and GST-AR, and GST protein alone were obtained by transforming expressing plasmids into BL21 (DE3) pLysS strain competent cells followed with 1 mM IPTG induction. GST-fusion proteins then were purified by Glutathione-Sepharose™ 4B (Pharmacia). The AR and Smad3 proteins labeled with [³⁵S] were generated in vitro by using the TNT-coupled reticulocyte lysate system (Promega). For the in vitro interaction, the glutathione-Sepharose bound GST-proteins were mixed with 5 μl of [³⁵S]-labeled TNT proteins in the presence or absence of 1 μM DHT at 4° C. for 3 h. The bound proteins were separated on an 8% SDS-polyacrylamide gel and visualized by using autoradiography.

(4) Mammalian Two-Hybrid Assay.

296. The mammalian two-hybrid system mainly followed the protocol of Clontech (California), with some modifications. Human prostate cancer DU145 cells were transiently co-transfected with Gal4-Smad3 expression plasmid, VP16-AR expression plasmid, and pG5CAT reporter plasmid in the presence or absence of 1 nM DHT. CAT assays were performed as described above.

(5) Co-immunoprecipitation of AR and Smads.

297. PC-3 Cells were co-transfected with AR and FLAG-Smad3 for 16 h, and then treated with vehicle or 10 nM DHT for another 16 h. LNCaP and PC3(AR)2 cells were treated with vehicle or 10 nM DHT for 16 h. The cells were lysed and incubated with monoclonal anti-FLAG antibody (Sigma), polyclonal Smad3 antibody (Santa Cruz), or control IgG at 4° C. for 2 h depending on the experimental design, followed by addition of protein A/G beads (Santa Cruz) for 1 h at 4° C. The bound proteins were separated on an 8% SDS-polyacrylamide gel and blotted with polyconal AR antibody (NH27), Smad3 antibody or anti-FLAG antibody. The bands were detected using an alkaline phosphatase detection kit (Bio-Rad).

(6) Northern Blot Analysis.

298. The blot containing approximately 20 μg of total RNA from LNCaP cells was transfected with Smad3 for 16 h, followed by DHT treatment for another 16 h. PSA expression level was determined by hybridizing with a probe from exon 1 of the PSA gene and labeled with [α-³²P] dCTP. A β-actin probe was used as a control for equivalent RNA loading.

### 4. Example 4

### PTEN Tumor Suppressor Interacts with and Promotes Degradation of Androgen Receptor

299. References cited in this example not specifically designated can be found in reference list C. Thus, a number designation of the reference refers to that number reference in Reference C.

a) Results

(1) PTEN Suppresses AR Transactivation Involving the Pathway Other than PI3K/Akt.

The effect of the PTEN on AR transactivation using mouse mammary tumor virus-luciferase (MMTV-luc) as an AR reporter was determined. PTEN repressed AR transactivation 4 fold in AR-positive LNCaP cells, but only less than 2 fold in AR negative DU145 and PC-3. PTEN C124S, a PTEN mutant without phosphatase activity (Wu et al. (1998) Proc Natl Acad Sci USA 95, 15587-91; Furnari et al. (1998) Cancer Res 58, 5002-8; Furnari et al. (1997) Proc Natl Acad Sci USA 94, 12479-84; Myers et al. (1998) Proc Natl Acad Sci USA 95, 13513-8), only showed a slight effect on AR transactivation in all cells tested (FIG. 20A). A similar result was obtained when the effect of PTEN on AR endogenous target gene by Northern blot analysis was tested. As shown in FIG. 20B, androgen induced the prostate-specific antigen (PSA) mRNA expression in LNCaP cells, and this induction was subsequently repressed by PTEN. However, PEN C124S only showed a slight inhibition. The inability of PTEN C124S to repress AR transactivation (FIG. 20A and 20B) indicates the phosphatase activity is important for PTEN function.

Two different promoters containing androgen response elements (AREs) [MMTV 5′ promoter and 4 copies of synthetic AREs (ARE)4] were used in a reporter gene assay to determine whether the effects of PTEN on the AR transactivation depend on the PI3K/Akt pathway. As shown in FIG. 20C, PTEN suppressed AR transactivation in both luciferase reporters at a similar level. The constitutively active form of Akt (cAkt) (Burgering et al. (1995) Nature 376, 599-602; Crowder et al. (1998) J Neurosci 18, 2933-43), like PTEN, could also suppress the AR transactivation (FIG. 20C). In contrast, a dominant-negative Akt (dakt) (Crowder et al.) and LY294002, a PI3K inhibitor, enhanced AR transactivation in both MMTV-Luc and (ARE)4-Luc. However, cAkt only slightly reversed the PTEN effect on AR transactivation, suggesting that the pathway other than PI3K/Akt is also involved (FIG. 20C). The Northern blot assay further confirmed this result. As shown in FIG. 20D, 10 nM DHT induced PSA mRNA expression in LNCaP cells and addition of LY294002 slightly enhance PSA mRNA, expression. Similar to the reporter gene assay, cAkt did not significantly reverse the PTEN effect on androgen-induced PSA expression (FIG. 20D). For controls, the potential effects of dAkt, cAkt, and LY294002 on the estrogen receptor (ER) transactivation were tested. Results from FIG. 20E showed that cAkt enhanced ER transactivation and dAkt as well as LY294002 suppressed ER transactivation. As these results are in agreement with a recent publication (Campbell et al. (2001) J Biol Chem 276, 9817-24), this can also indicate that the reagents (cAkt, dAkt, and LY294002) used were correct. Addition of cAkt with PTEN only slightly relieved PTEN-suppressed AR transactivation (FIG. 20C), indicates the relevant PTEN-AR pathway is different.

(2) PTEN Interacts with AR in vitro and in vivo.

300. To further dissect the potential mechanisms by which PTEN can suppress AR transactivation, PTEN's ability to directly interact with AR wwas tested. The glutathionine-S-transferase (GST) pull-down assay results indicated that PTEN could interact with AR in the presence or absence of androgen (FIG. 21A). Among several nuclear receptors tested, it was found that PTEN binds preferentially to AR and the ER, but not to the glucocorticoid receptor (GR), the progesterone receptor (PR), or the retinoid-X receptor (RXR) (FIG. 21A).

301. To map the AR interaction domains, a set of serial deletions of PTEN were constructed for the GST pull-down assays. The AR was able to interact with GST-PTEN-#2 (aa, 107-252), where the phosphatase domain is located, but not with GST-PTEN-#1 (aa, 1-107) or GST-PTEN-#3 (aa, 253-403) (FIG. 21B & 21C). Further peptide mapping revealed that the peptide (aa, 110-163, named as PTEN-PTP) containing the phosphatase domain, could interact well with AR (FIG. 21C).

302. Studies of the PTEN interacting domain on AR indicated that the AR-DBD (aa, 486-651), and AR-DBD plus LBD (AR-DBD-LBD) (aa, 552-918), were able to bind PTEN, but not the AR amino-terminal region (AR-N) (aa, 34-560) or AR-LBD (aa, 666-918) (FIG. 21D & 21E). These GST pull-down assay results can therefore indicate that the phosphatase domain within PTEN interacts with the AR in the DBD and hinge region (aa, 552-651). N-terminal-taged six histidines in front of AR-N-terminal plus DBD (His-AR-N-DBD) were used to examine whether it can bind to soluble PTEN or PTEN C124S. The His-AR-N-DBD, which contains DBD that binds to PTEN, was expressed in bacteria, purified with the nickel (Ni²⁺) column, and incubated with the in vitro expressed [³⁵S]-PTEN and [³⁵S]-PTEN C124S. As shown in FIG. 22F, the PTEN could bind AR-N-DBD strongly, whereas the PTEN mutant dramatically decreased the ability to interact with AR-N-DBD. These results correlated well with the suppression of AR transactivation by PTEN, but not by PTEN C124S (FIG. 20A), and suggested that interaction between PTEN and AR might play important roles in the PTEN-mediated suppression of AR transactivation. As the phosphatase region of PTEN is frequently mutated in primary tumors (Lee et al. (1999) Cell 99, 323-34), mutations in this region can result in the loss of its ability to bind to and modulate AR, which can then contribute to the deregulated cancer growth.

The interaction between AR and PTEN was further confirmed by co-immunoprecipitation. For this purpose, it was established PTEN-stable LNCaP cells by doxycycline (Dox)-inducible system. Dox treatment induced expression of PTEN and PTEN C124S in several clones (PTEN-C1, PTEN-C2, PTEN C124S-C4, and PTEN C124S-C8, FIG. 22A). FIG. 22B showed that PTEN could be co-immunoprecipitated with AR in PTEN-C1 cells. To rule out the possibility that PTEN antibody may cross-react with AR, LNCaP cells, which express AR but not PTEN, were applied to demonstrate that PTEN antibody did not pull-down AR (data not shown). To further prove the endogenous PTEN can interact with the endogenous AR in the prostate cancer cell line, the CWR22 cell line (Amler et al. (2000) Cancer Res 60, 6134-41; McDonald et al. (2000) Cancer Res 60, 2317-22), an androgen-dependent cell line isolated from human prostate tumors, which expresses both AR and PTEN (FIG. 22C), was applied for co-immunoprecipitation. The results showed that AR was detectable in the PTEN immunoprecipitated complex (FIG. 22C) and PTEN could also be detected in the AR immunoprecipitated complex (FIG. 22D). These results indicate that endogenous PTEN can interact with endogenous AR under the physiological condition in prostate cancer cells. PTEN and AR interaction was further confirmed by colocalization studies. As shown in FIG. 23A, the fluorescent FITC stained PTEN was mainly located in the cytosol, but small amounts of PTEN were also found in the nucleus. Similar to the FITC stained PTEN, Texas-RED stained AR was also mainly located in the cytosol in the absence of androgen, but androgen treatment caused AR nuclear translation (FIG. 23A). FIG. 23B further demonstrated that PTEN could colocalize with AR in the presence or absence of androgen. Together, the co-immunoprecipitation, co-localization assay, and GST-pull-down assay demonstrate that PTEN can interact with AR.

(3) PTEN Decreases AR Protein Levels Via Promotion of AR Degradation.

303. Results from transient transfection and Western blot analysis showed that PTEN could reduce AR protein levels in COS-1 cells (FIG. 24A) indicating PTEN effects AR protein stability. To rule out the possibility that PTEN can influence the expression of exogenously transfected AR construct through the promoter region, the expression of endogenous AR in PTEN-stable LNCaP cells was tested. As shown in FIG. 24B, Dox-induced expression of PTEN in LNCaP PTEN-C1 and PTEN-C2 reduced endogenous AR protein levels. In contrast, Dox-induced PTEN C124S expression (PTEN-C124S-C4 and PTEN-C124S-C8) failed to reduce endogenous AR protein levels. Together, the data clearly demonstrate that PTEN can interact with AR and reduce AR protein levels in COS-1 and LNCaP cells. To determine if reduced AR protein levels were due to reduced mRNA expression, a portion of each LNCaP cell lysate was saved for Northern blot analysis. As shown in FIG. 24C, while AR protein levels were reduced by Dox-induced PTEN, the AR mRNA levels normalized by β-actin remained relatively unchanged, indicating that PTEN can reduce the AR protein levels through post-transcriptional modification.

304. Pulse-chase labeling was used to study AR protein stability. As shown in FIG. 24D, PTEN clearly reduced the half-life of newly synthesized [³⁵S]-AR 4 to 5-fold and accelerated AR degradation. Interestingly, when PTEN was replaced with either dAkt or LY294002, the results (FIG. 24E) indicated that dAkt and LY294002 did not promote AR degradation. These data strongly indicate that PTEN-AR direct protein-protein interaction can play major roles for the PTEN-promoted AR degradation.

(4) Interaction Between PTEN and AR Contributes to PTEN-induced Suppression of AR Functions and Apoptosis.

305. To further prove PTEN suppression of AR function can go through direct PTEN-AR interaction, the interaction domain within AR (aa; 483-651, named as ARf) was used for functional studies. To determine whether ARf could disrupt the interaction between AR and PTEN, a co-immunoprecipitation assay was preformed. As shown in the FIG. 22E, ARf interacted with PTEN and disrupted the interaction between AR and PTEN in the CWR22 cells. The results further showed that ARf could dramatically reduce PTEN-mediated promotion of AR degradation (FIG. 25A) and PTEN-mediated suppression of AR transactivation (FIG. 25B), indicating that PTEN and AR interaction plays important roles for the PTEN effect on suppression of AR transactivation and promotion of AR degradation. To extend the studies of PTEN on the suppression of AR function, another prostate cancer cell line, CWR22, which expresses functional AR and is growth-dependent on androgen (Amler et al. (2000) Cancer Res 60, 6134-41; McDonald et al. (2000) Cancer Res 60, 2317-22) was used. As shown in FIG. 25C, PTEN dramatically suppressed AR transactivation. Remarkably, ARf could significantly reduce PTEN suppressive effect on AR transactivation (FIG. 25C). These results therefore are in agreement with the results from LNCaP cells (FIG. 25B) and suggest that PTEN can modulate AR functions by its interaction with AR in the various stages of prostate cancers.

306. The PTEN tumor suppressor induces cell apoptosis in a variety of cell types including the LNCaP cells. It was hypothesized that suppression of AR activity by PTEN contributes to PTEN-induced apoptosis. To test this hypothesis, the effect of ARf, which could relieve the suppressive effect of PTEN on AR transactivation (FIG. 25B & 25C), on PTEN-induced apoptosis in LNCaP cells by TUNEL assay was studied. As expected, PTEN could induce apoptosis markedly, whereas PTEN C124S showed only a marginal effect (FIG. 25D). ARf markedly reduced PTEN-induced apoptosis (FIG. 25D). The suppressive effect of ARf on PTEN functions was not due to the interference of PTEN phosphatase activity, since ARf showed little influence on the PTEN-mediated inhibition of Akt activity (FIG. 25E). Together, these data clearly indicate that interaction between PTEN and AR contributes to PTEN-induced suppression of AR functions and apoptosis. The results (FIG. 25D) also confirmed an earlier report (Myers et al., 1998) that PTEN-induced cell death could be suppressed by adding cAkt, indicating that the PTEN→PI3K→Akt pathway can also play roles in the mediation of PTEN-induced cell death.

307. The PTEN-PTP, which interacts with AR, was included to test its effect on the regulation of AR function. It was found that PTEN-PTP, like the full length PTEN, could promote AR degradation (FIG. 25A) and suppress of AR transactivation (FIG. 25B & 25C). In contrast, PTEN-C124S or PTEN-#1 peptide that does not interact with AR (FIG. 21C), showed only marginal effect on those PTEN functions (FIG. 25A & 25B). These results indicate that interaction of PTEN with AR plays an important role for PTEN action.

308. To rule out the possibility that ARf may have nonspecific effects, glioblastoma U87MG cells were used to test the effects of ARf on the PTEN-induced apoptosis in AR-negative cells. Both Western blot assay and AR transactivation assay indicated that AR was undetectable in U87MG cells (data not shown). While PTEN was still able to induce apoptosis in AR-negative U87MG cells, addition of ARf, however, showed only marginal effects on the PTEN-induced apoptosis (FIG. 25F). However, cAkt was able to suppress PTEN-induced apoptosis (FIG. 25F). These results indicate that the effect of ARf on PTEN-induced apoptosis is not nonspecific and requires the intact AR signaling. Together, results from FIG. 6 clearly indicate that PTEN can have two distinct pathways (PTEN→PI3K/Akt and PTEN→AR) to induce apoptosis and interaction of PTEN with AR can play important roles in one of these two pathways in the LNCaP prostate cancer cells.

b) Materials and Methods

(1) Material

309. pCDNA3 cAkt and pCDNA3 dAkt were from Dr. R. Freeman. LY294002 was from Calbiochem. 5α-dihydrotestosterone (DHT), doxycycline, and MTT were from Sigma. The anti-AR polyclonal antibody, NH27, was produced as previously described (Yeh et al. (1996) Proc Natl Acad Sci USA 93, 5517-21; 162; Yeh et al. (1998) Biochem Biophys Res Commun 248, 361-7). PTEN monoclonal antibody was from Santa Cruz. FITC-conjugated secondary anti-mouse antibody and Texas-RED-conjugated secondary anti-rabbit antibody were from ICN. TNT in vitro protein expression kit was from Promega. pCDNA3-PTEN and pCDNA3-PTEN C 124S were provided by Dr. C. L. Sawyers. pSG5-HA-PTEN was from Dr. W. Sellers. PTEN-#1 and ARf were constructed into pCMV-Flag vector, and PTEN-PTP was constructed in pCMV-HA vector using the polymerase chain reaction (PCR) method. To construct GST-PTEN fragment proteins, appropriate restriction enzymes were used to release PTEN fragments (#1, #2, #3) from pGEM-KG-PTEN (from Dr. F. Furnari) and subcloned into pGEM-KG, pGEX-3×, and pGEX-3× (Amersham Pharmacia), respectively. To construct GST-PTEN-PTP, the PTP fragment was obtained by PCR and inserted into pGEX-3×.

(2) Cell Culture and Transfections

310. The DU145, PC-3, and COS-1 cell lines were maintained in Dulbecco's Minimum Essential Medium (DMEM) containing penicillin (25 U/ml), streptomycin (25 μg/ml), and 10% fetal calf serum (FCS). The LNCaP, U87MG, and CWR22 (a gift from Dr. C. Kao) cells were maintained in RPMI-1640 with 10% FCS. Transfections were performed using the calcium phosphate precipitation method in PC-3 and DU145 cells, as previously described (Yeh et al. (1999) Proc Natl Acad Sci USA 96, 5458-63.) or SuperFect™ in LNCaP, COS-1, and U87MG cells according to standard procedures (Qiagen).

(3) Apoptosis Assay

311. For the apoptosis assay, the cells were transfected with plasmids for 24 h and grown in 0.1% charcol-stripped serum (CDS) media. The apoptosis was determined 2 days after transfection using the TUNEL assay according to the standard procedures (Oncogene). At least 200 cells were scored for each sample and data are means±s.d. from three independent experiments.

(4) Luciferase Reporter Assays

312. The cells were transfected with plasmids in 10% CDS media for 16 h and then treated with ethanol or 10 nM DHT for 16 h. The cells were lysed and the luciferase activity was detected by the dual luciferase assay according to standard procedures.

(5) GST Pull-down Assay

313. GST fusion proteins were purified as described by the manufacturer (Amersham Pharmacia). The purified GST-proteins were resuspended with 100 μl of interaction buffer (20 mM Tris-HCl/pH 8.0, 60 mM NaCl, 1 mM EDTA, 6 mM MgCl₂, 1 mM Dithiothreitol, 8% Glycerol, 0.05% (v/v) NP-40, 1 mM PMSF, and proteinase inhibitors) and mixed with 5 μl of [³⁵S]-labeled TNT proteins in the presence or absence of 1 μM ligands on a rotating disk at 4° C. for 2 h. After extensive washes with NENT buffer, the bound proteins were separated on an 8% SDS-polyacrylamide gel and visualized by autoradiography.

(6) Immunoprecipitation and Western Blot Analysis

314. The immunoprecipitation and Western blotting were performed as previously described (Qiu et al. (1998) Nature 393, 83-5). The cell extracts (1 mg) were immunoprecipitated with the indicated antibody. The immunocomplexes were subjected to 8% SDS-PAGE and immunoblotted with the indicated antibody.

(7) Immunofluoresence and Microscopy

315. The COS-1 cells were plated on 12-mm coverslips and incubated overnight, transfected with pSG5-AR in combination with pCDNA3, pCDNA3 PTEN, or pCDNA3 PTEN C124S in 10% CDS media for 16 h, and then treated with ethanol or 10 nM DHT for another 16 h. The cells were fixed with 4% paraformaldehyde/PBS for 20 min on ice and cells were permeabilized with 100% methanol for 15 min on ice. The following experiments were performed at room temperature. The coverslips were rinsed with PBS twice and incubated in 5% bovine serum albumin (BSA) for 30 min. The primary antibodies against AR and PTEN were added for 1 h and washed with PBS 4 times. The secondary antibodies were added for 1 h and then washed 4 times with PBS, followed by application of the counting medium containing 4,6-diaminodino-2-phenylinodel (DAPI). A FITC-conjugating anti-mouse antibody and a Texas-RED anti-rabbit antibody were used as secondary antibodies. Coverslips were examined by confocal microscope.

(8) LNCaP Stable Transfections

316. For the Dox-inducible system, PTEN or PTEN C124S were released from pGEM-KG-PTEN or pGEM-KG-PTEN C124S using EcoRI digestion and inserted into pBIG2i vector. The LNCaP cells were transfected with pBIG2i vector, pPIB2i PTEN, or pBIG2i PTEN C124S for 24 h. The cells were selected using 100 μg/ml hygromycin. Individual single colonies were picked and grown until 70% confluent, followed by 4 μg/ml Dox treatment for 48 h. The positive clones were confirmed by Western blot analysis.

(9) Pulse-chase Experiments

317. Pulse-chase experiments were performed as described (Lo et al. (1999) Nat Cell Biol 1, 472-8) with some modifications. Briefly, COS-1 cells were transfected with pSG5-AR in combination with pCDNA3 or pCDNA3 PTEN in 10% CDS media for 36 h. Cells were grown under serum starvation conditions for 2 h in methionine/cysteine-deficient medium, and then the cells were pulsed for 45 min with 200 μCi/ml [³⁵S]-methionine/cysteine (NEN). Cells were washed with DMEM twice and incubated with DMEM containing 0.2% CDS along with 10 nM DHT. The cells were lysed by RIPA buffer in the presence of protease inhibitors, followed by immunoprecipitation using AR antibody. The immunocomplexes were subjected to 8% SDS-PAGE, and visualized by autoradiography.

### 5. Example 5

### Differential Modulation of Androgen Receptor Transactivation by the Interaction with Smad3 and Tumor Suppressor Protein Smad4

318. References cited in this example not specifically designated can be found in reference list D. Thus, a number designation of the reference refers to that number reference in Reference D.

a) Results and Discussion

(1) Repression of Smad3-enhanced AR Transactivation by Smad4 in Different Prostate Cancer Cells

319. To study the potential correlation between AR and Smads in prostate cancer cells, prostate cancer PC3-AR2 cells, which were stably transfected with wild type AR (wtAR), were chosen to examine the effect of Smads on androgen-mediated mouse mammary tumor virus (MMTV) promoter activity. Activation of MMTV-CAT activity was achieved by treating with 10⁻⁸ M DHT (FIG. 26A, lane 1 vs. 2) and this androgen-activated transactivation was further enhanced by the addition of Smad3, but not Smad4 (FIG. 26A, lane 3 and 4 vs. 5 and 6). Interestingly, Smad3-enhanced AR transactivation was significantly repressed by adding Smad4 (FIG. 26A, lane 3 vs. 7). Similar results were obtained with LNCaP cells that expressed mutated but functional AR (FIG. 26A). Next, we examined the effect of Smads on AR transactivation by increasing the dose of Smad3, Smad4, and SRC-1 in PC3-AR2 cells. As shown in FIG. 26B, in the presence of Smad3, the addition of Smad4 can result in the suppression of Smad3-enhanced AR transactivation in a dose-dependent manner (FIG. 26B, lane 3 vs. 4, 5, and 6). In the presence of Smad4, AR transactivation was slightly suppressed (FIG. 26B, lane 2 vs. 10) and adding Smad3 could then further suppress AR transactivation (FIG. 26B, lane 10 vs. 11, 12, and 13). Interestingly, in the presence of Smad3 or Smad4, addition of SRC-1 can then increase AR transactivation (lane 3 vs. 7, 8, 9 and lane 10 vs. 14, 15, and 16). Together, these results from reporter assays suggested that the amount of Smad4 available in the cell might have a major influence on the Smad3 effect on the AR transactivation. Whether there is enough Smad4 available in the cells to heterodimerize with Smad3 determines if Smad3 can induce or suppress AR transactivation.

(2) Suppression of Endogenous AR target gene PSA mRNA Expression by Smad3/Smad4

320. To further confirm Smad3/Smad4 suppression effects on AR transactivation and reduce potential artifact effects related to reporter assays, we then applied Northern blot to assay Smad3/Smad4 suppression effect on AR endogeneous target gene, PSA mRNA expression. In LNCaP cells, 10 nM DHT increases PSA mRNA expression (FIG. 26C, lane 1 vs. 2). Addition of Smad3 further enhances DHT-induced PSA mRNA expression (FIG. 26C, lane 2 vs. 3), whereas addition of Smad4 alone has slight suppression of PSA mRNA expression (FIG. 26C. lane 2 vs. 3). Addition of both Smad3 and Smad4 reverses the Smad3-enhanced PSA mRNA expression. Similar results were also obtained with the real-time quantitative RT-PCR assay to measure PSA mRNA expression (Table I ands II and FIG. 26D). Together, FIG. 26C and 26D, all confirmed the above ARE-reporter assays showing that addition of Smad3 alone can enhance AR transactivation and addition of both Smad3 and Smad4 can then suppress AR transactivation.

(3) Interaction Between AR, Smad3, and Smad4 in vitro and in vivo

321. N-terminal Flag-tagged, full-length Smad3 and Smad4 were expressed in PC3-AR2 cells and treated with DHT (or vehicle). Cell extracts were prepared for immunoprecipitations using anti-Flag or anti-AR antibodies. As shown in FIG. 27A, in the presence of Flag-Smads, AR was co-immunoprecipitated with Smads both in the presence or absence of 10 nM DHT. Moreover, FIG. 27B demonstrated that immunoprecipitating against AR can also capture Smads/AR complexes independent of DHT addition. An in vivo co-immunoprecipitation assay was further applied to demonstrate that the endogenous Smad4 immunocomplex in the absence or presence of DHT in PC3-AR2 cells, but not in AR-negative PC-3 or DU145 cells. A similar result was also obtained when we replaced PC3-AR2 with LNCaP cells.

322. To dissect which individual domain of AR can interact with Smad4, GST-Smad4 fusion proteins incubated with various AR deletion mutants in pull-down experiments were used. As shown in FIG. 28A, the full-length wtAR, AR-DBD/LBD peptides, and the AR-DBD and AR-LBD peptides, but not the N-terminal AR peptide, could interact with Smad4. Similar results were obtained with GST-AR, which can also bind to Smad3 or Smad4 (FIG. 28B). These results suggest that both DBD and LBD domains of AR may contain binding sites for Smad4 or Smad3 interactions.

323. In addition, when GST-Smad3 was incubated with AR and different amounts of Smad4 were added to this protein complex, the full-length wtAR could interact with Smad3. Interestingly, addition of Smad4 can decrease this AR-Smad3 interaction in a dose-dependent manner (FIG. 28C). Previous reports showed that Smad3 can interact with Smad4 (Derynck et al. (1998) Cell 95, 737-740; Zhang et al. (1996) Nature 383, 168-172; Wrana (1998) Miner Electrolyte Metab. 24, 120-130) and results disclosed herein further demonstrated that both Smad3 and Smad4 can interact with AR in the DBD and LBD. It is possible that Smad4 may either compete directly with Smad3 for the same AR binding sites or Smad4 may have a higher binding affinity then Smad3 to bind to AR.

324. Next, we examined which region(s) within the Smad4 are important for repression of Smad3-enhanced AR transactivation. As shown in FIG. 4B, addition of Smad3 increased the AR transactivation (lane 2 vs. 3), co-transfection of Smad4 and Smad3 can then repress the Smad3-enhanced AR transactivation (lane 3 vs. 7) in PC-3 cells. In contrast, replacing Smad4 with a Smad4 mutant, with a C-terminal deletion, can only show partial suppression effect (lane 7 vs. 8). On the other hand, a C-terminal deletion of Smad3 resulted in the loss of the

325. Smad3 enhanced effect on the AR transactivation (lane 3 vs. 5). As previous reports indicated that the MH2 region of the C-terminal Smad proteins is important for homo-oligomerization and hetero-oligomerization (11), it is possible that the C-terminal region is also important for Smad proteins to interact with AR and exert its function on the modulation of AR transactivation.

326. To further dissect the repression domains within the C-terminal region of Smad4, a series of deletion mutants of Smad4 (FIG. 4B), were constructed and tested in the Smad3-enhanced AR transactivation. As shown in FIG. 29C, among all mutants, ΔM4 with a deletion of aa 274-322, has the greatest effect to reverse the Smad4 suppression of Smad3-enhanced AR transactivation, suggesting that the region (aa 274-322) within the C-terminal of Smad4 may play essential roles to repress Smad3-enhanced AR transactivation.

(4) Transcriptional Repression by Smad3/Smad4 is Associated with Decreasing AR Acetylation

327. Previously, p300/CBP was shown to be able to acetylate non-histone proteins including AR and the addition of trichostatin A (TSA), a specific inhibitor of histone deacetylase activity, was shown to induce the androgen-mediated AR transactivation (Fu et al. (2000) J Biol. Chem. 275, 20853-20860; List, et al. (1999) Exp. Cell Res. 252, 471-478; Sharma et al. (2001) Mol. Endocrinol. 15, 1918-1928). Other reports also suggested that some Smads could interact directly with several HDAC1-associated proteins, such as HDAC-1, TGIF, c-ski, and SnoN (Wotton et al. (2001) Curr Top Microbiol Immunol 254, 145-164; Leong et al. (2001) J. Biol. Chem. 276, 18243-18248; Liberati et al. (2001) J. Biol. Chem. 276,22595-22603; Xu et al. (2000) Proc. Natl. Acad. Sci. USA 97, 5924-9), which might play roles in the Smads-mediated transcriptional suppression. Whether repression of AR by Smad3/Smad4 might involve the acetylation of AR, however, remains unknown. It was determined whether TSA had any influence on the Smad3/Smad4-mediated AR transactivation. As shown in FIG. 30A, addition of TSA can enhance AR transactivation in a dose-dependent manner (lane 2 vs. lane 5, 10, and 14). More importantly, addition of TSA can also reverse the Smad3/Smad4-suppressed AR transactivation in a dose-dependent manner in PC3-AR2 cells (lane 3 vs. 7, 11, and 15). A similar effect was also observed when cells were treated with NaB, another specific inhibitor of histone deacetylase. To further determine whether Smad3 and Smad4 could influence the acetylation of AR, immunoprecipitation was performed using an AR-specific antibody on cell extracts derived from PC2-AR2 cells transfected with different Smad expression vectors. The immunoprecipitate was subjected to Western blotting with an anti-acetyl-lysine antibody, AR-specific antibody, or anti-Flag antibody. As shown in FIG. 30B, acetyl-lysine-immunoreactive bands were detected in the AR antibody IP complex, which have the identical mobility to the AR bands, whereas acetyl-lysine-immunoreactivity was decreased when cells were co-transfected with Smad3 and Smad4. Together, these results suggest that Smad3/Smad4 may modulate the endogenous deacetylase that results in the decrease of AR acetylation. The consequence of these events may then result in the suppression of AR transactivation.

(5) The Effects of Smads on AR Transactivation is Promoter Context and Cell Type Dependent

328. Hayes et al. reports that addition of Smad3 in PC-3 and CV1 cells can suppress AR transactivation (Hayes et al. (2001) Cancer Res. 61, 2112-2118), which is in contrast with our earlier report showing Smads can induce AR transactivation in DU145, LNCaP, and SW480·C7 cells (Kang, et al. (2001) Proc. Natl. Acad. Sci. USA 98, 3018-3023). As shown in FIGS. 26 and 27, addition of Smad3 alone can enhance AR transactivation and adding both Smad3 and Smad4 can then suppress AR transactivation in LNCaP and PC3-AR2 cells. It is possible that the amount of endogenous Smad3 and/or Smad4 available in the tested cells may influence the heterodimerization between Smad3 and Smad4. How Smad3/Smad3 and Smad4/Smad4 homodimers as well as Smad3/Smad4 heterodimer interact with the ARE-promoter linked to the reporter could also influence the AR transactivation. In PC-3 cells, two different reporters linked with AR target gene natural 5′ promoters (MMTV and PSA) and two different synthetic AREs (5X-ARE and TAT2-ARE) were tried, and results show that adding Smad3 alone can enhance all these reporters linked with the four different AREs, and adding Smad3/Smad4 together can then reverse these enhancement effects (FIG. 31A). In contrast, when PC-3 cells were replaced with SW480·C7 cells, a cell line deficient in Smad4 (Goyette et al. (1992) Mol. Cell. Biol. 12, 1387-1395), we found that adding Smad3 alone can only enhance MMTV-ARE reporter activity (FIG. 31B, lane 2 vs 3). For the other 3 ARE reporters (PSA-ARE, 5X-ARE, and TAT2-ARE), Smad3 alone has some slight repression (FIG. 31B, lane 8 vs. 9, 14 vs. 15, and 20 vs. 21). Again adding Smad4 and Smad 3 together can then suppress AR transactivation in all these 4 different ARE reporters (FIG. 31B, lane 2 vs. 5, 8 vs. 11, 14 vs. 17, and 20 vs. 23).

329. These results, together with previous reports (Hayes et al. (2001) Cancer Res. 61, 2112-2118; Kang et al. (2001) Proc. Natl. Acad. Sci. USA 98, 3018-3023), suggested that the suppression effect by Smad3/Smad4 is consistent and not dependent on either different ARE-promoters or cell type. In contrast, the ability of Smad3 to modulate AR transactivation not only depended on how much it can heterodimerize with Smad4, but may also depend on the context of the ARE promoter and cell type. The detailed mechanisms of how Smad3 and/or Smad4 can modulate AR transactivation, however, remains unclear. One possible mechanism to explain how Smad3/Smad4 can modulate AR transactivation is that after interaction with AR, the complex of Smad3/Smad4/AR may be able to recruit some transcriptional repressors, which may then result in the suppression of AR transactivation via decreasing the acetylation level of AR (FIG. 30). As the p300 coactivator may compete with some HDAC1-associated proteins, such as c-ski or TGIF, for the binding to the MH2 domain of Smad3 (Fu et al. (2000) J. Biol. Chem. 275, 20853-20860) and TGIF have been demonstrated to interact with AR and Sin3A (Luo et al. (1999) Genes Dev. 13, 2196-2206), it will be interesting to test if the c-ski or TGIF has any influence on the Smad3/Smad4-mediated suppression of AR transactivation.

330. Furthermore, our data indicated that the MH2 domain within the C-terminal of Smad4 plays important roles to reverse the Smads-enhanced AR transactivation and early reports suggested that the MH2 domain in the Smads played important roles for the homo- or hetero-oligomerization (1-3). Therefore it is possible that Smad3 may either homodimerize with Smad3 or heterodimerize with Smad4 via their MH2 domains. The consequence of such homo- or hetero-oligomerizations can then result in either the induction or suppression of AR transactivation.

331. Together, FIG. 32 demonstrates a simple model for the modulation on AR mediated-transactivation by Smad3/Smad4. First, Smad3 may function as a positive coregulator to enhance AR transactivation. As both Smad3 and Smad4 can interact with AR-DBD and AR-LBD, it is likely that Smad4 may directly compete with Smad3 to bind to AR or Smad 3 may have a higher binding affinity to Smad4, as compared to AR binding. The consequence of such multiple interactions among Smad3, Smad4, and AR may then weaken the smad3 coactivator activity tp enhance AR transactivation. Further studies to determine how the control of promoter sequences can influence Smad3 modulation of AR transactivation, and how the ratio between Smad3:Smad4:AR can influence Smads modulation of AR transactivation, may help us to better understand the potential roles of Smad3 and Smad4 in the AR-mediated prostate cancer progression.

b) Materials and Methods

(1) Chemicals and Plasmids.

332. DHT and hydroxyflutamide from Schering, USA. pSG5-wild type AR (wtAR) and pCMV-AR were used in our previous report (Fu et al. (2000) J. Biol. Chem. 275, 20853-20860). Expression plasmids for glutathione S-transferase (GST)-Smad3 and Smad4 and full-length cDNAs of human Smad3 and Smad4, were kindly provided by Rik Derynck (Zhang et al. (1996) Nature 383, 168-72). Smad4 (G508S), Smad4 (D539H), Smad4 ΔM1, ΔM2, ΔM3 and ΔM4 were provided by Dr. Mark P. de Caestecker. (de Caestecker et al. (2000) J. Biol. Chem. 275, 2115-2122; de Caestecker et al. (1997) J. Biol. Chem. 272, 13690-13696.

(2) Cell Culture and Transfections

333. Human prostate cancer DU145 cells and PC-3 cells were maintained in Dulbecco's Minimum Essential Medium containing penicillin (25 U/ml), streptomycin (25 μg/ml), and 5% fetal calf serum. Transfections were performed using the calcium phosphate precipitation method and cells were harvested after 24 h for the chloramphenicol acetyltransferase (CAT) assay, as described previously (Kang, et al. (1999) J. Biol. Chem. 274, 8570-8576). The CAT activity was visualized and quantitated by STORM 840 (Molecular Dynamics). At least three independent experiments were carried out in each case. The SW480·C7 cells and PC-3(AR)2 cells are the gifts from Dr. Eric J. Stanbridge and Dr. T. J. Brown

(3) Western Blot Analysis

334. Cells were lysed in lysis buffer (50 mM Tris-HCl/pH 7.4, 150 mmol/l NaCl, 1 mmol/l EDTA, 1% NP-40, 0.25% sodium deoxycholate, 1 mM phenylmetlhylsulfonyl fluoride, 1 μg/ml aprotinin, 1 μg/ml leupeptin, 1 μg/ml pepstatin). An aliquot of each sample was used for determination of protein content using the Bradford protein assay reagent. The clarified supernatants were mixed (1:4) with 5× sample buffer (10% sodium dodecyl sulphate (SDS), 0.5M Tris/pH 6·8, 10% glycerol, 1% bromophenol blue, 5% β-mercaptoethanol), transferred to a boiling water bath for 5 min, rapidly frozen on dry ice, and stored at −70° until use. Samples with equivalent amounts of protein were subjected to SDS-PAGE on a 7.5% acrylamide gel. Proteins were transferred to Hybond-P membrane (PVDF transfer membrane) at 100 V, 90 mins. After blocking in TBST buffer (10 mmol/l Tris HCL/pH 7·5, 150 mmol/l NaCl, 0·05% Tween 20) supplemented with 5% non-fat dry milk, blots were incubated with the primary mouse AR antibodies (1:1000 dilution) or anti-flag antibodies (1:1000 dilution), in TBST for 90 min. To demonstrate androgen receptor or anti-flag, blots were incubated with horseradish peroxidase-labeled anti-mouse immunoglobulins for 1 h. Films were developed using the ECL Western blot analysis system from Amersham Pharmacia Biotech.

(4) GST Pull-down Assay

335. Fusion proteins of GST-Smad3 and GST-AR, and GST protein alone were obtained by transforming expressing plasmids into BL21 (DE3) pLysS strain competent cells followed with 1 mM IPTG induction. GST-fusion proteins then were purified by Glutathione-Sepharose™ 4B (Pharmacia). The wtAR and deletion mutant AR (mtAR) proteins labeled with [³⁵S] were generated in vitro using the TNT-coupled reticulocyte lysate system (Promega). For the in vitro interaction, the glutathione-Sepharose bound GST-proteins were mixed with 5 μl of [³⁵S]-labeled TNT proteins in the presence or absence of 1 μM DHT at 4° C. for 3 h. The bound proteins were separated on an 8% SDS-polyacrylamide gel and visualized by using autoradiography.

(5) Co-immunoprecipitation of AR and Smads

336. PC-3 Cells were co-transfected with AR and FLAG-Smad4 and Smad3 for 16 h, and then treated with vehicle or 10 nM DHT for another 16 h. LNCaP and PC-3(AR)2 cells were treated with vehicle or 10 nM DHT for 16 h. The cells were lysed and incubated with monoclonal anti-FLAG antibody (Sigma), polyclonal Smad4 and Smad3 antibodies (Santa Cruz), or control IgG at 4° C. for 2 h depending on the experimental design, followed by addition of protein A/G beads (Santa Cruz) for 1 h at 4° C. The bound proteins were separated on an 8% SDS-polyacrylamide gel and blotted with polyconal AR antibody (NH27), Smad4 and Smad3 antibodies or anti-FLAG antibody. The bands were detected using an alkaline phosphatase detection kit (Bio-Rad).

(6) Immunoprecipitation

337. PC-3(AR)2 and LNCaP Cells were grown in 100-mm culture dishes and treated with vehicle or 10 nM DHT for 24 h. The cells were lysed in EBC (150 mM NaCl, 50 mM Tris-HCl/pH 8.0, 0.5% NP-40, with protease inhibitors). Cell lysates were harvested, and 1 mg of total protein was precleared with 20 μl of protein A/G beads (Santa Cruz) for 2 h at 4° C. The beads were pelleted by centrifugation at 12,000 rpm for 30 sec, and the supernatants were subjected to immunoprecipitation with 5 μl of mouse anti-human AR antibody (BD PharMingen), and 30 μl of protein A/G beads at 4° C. for 2 h. The beads were pelleted by centrifugation; washed five times with NETN buffer (100 mM NaCl, 1 mM EDTA, 20 mM Tris-HCl/pH 8.0. 0.25% NP-40, with protease inhibitors), boiled with 30 μl of 6% SDS-polyacrylamide gel electrophoresis loading buffer for 5 min, and the immunoprecipitated proteins were separated by 10% SDS-polyacrylamide gel electrophoresis. Western blotting of the membrane was performed with the rabbit anti-Smad4 (H-552 Santa Cruz).

(7) Principle of Real-Time PCR.

338. The theoretical basis of the ABI PRISM 7700 Sequence Detection System real time quantitative PCR (Perkin-Elmer Applied Bio systems) is described elsewhere. Fluorescence signal; from each PCR reaction is collected as normalized values plotted versus the cycle number. Reactions are characterized by comparing threshold cycle (C_{T}) values. The C_{T} is a value define the fractional cycle number at which the sample fluorescence signal passes a fixed threshold above base. Quantitative values are obtained from the C_{T} number at which the increase in signal associated with an exponential growth of PCR product starts to be detected (using Perkin-Elmer Bio systems analysis software), according to the manufacturer's manual. The precise amount of total RNA added to each reaction (based on absorbance) and its quality (i.e., lack of extensive degradation) are both difficult to assess. Therefore, we also quantified transcripts of the gene β-actin as the endogenous RNA control, and each sample was normalized on the basis of its β-actin content. The relative target gene expression level was also normalized to the calibrator. The final results, expressed as N-fold differences in target gene expression relative to the β-actin gene and the calibrator termed “N target,” were determined as follows:

N target=2^{-(ΔCTsample-ΔCTcalibrator)}

where ΔC_{T} values of the sample and calibrator are determined by subtracting the average C_{T} value of the target gene from the average C_{T} value of the β-actin gene.

(8) Oligonucleotide Primers Design

339. The target cDNA sequence was evaluated using the Primer Express software (Perkin-Elmer Applied Bio systems) (table 1). The forward and reverse primers were designed to lie in adjacent exons to prevent amplification genomic DNA that may be contained in samples.

(9) RNA Extraction

340. Total RNA was extracted from LNCap cell lines by using the Trizol (GibcoBRL). The quality of the RNA samples was determined by electrophoresis through agarose gels and staining with ethidium bromide. The 18S and 28S RNA bands were visualized under UV light.

(10) cDNA Synthesis

341. RNA was reverse transcribed in a final volume of 20 ml containing 5×reverse transcriptase buffer (500 mM each dNTP, 3 mM MgCl₂, 75 mM KCl, and 50 mM Tris-HCl/pH 8.3), 0.1 μg of Oligo dT, 10 units of Rnasin inhibitor (Promega, Madison, Wis.), 100 units of MMLV reverse transcriptase (Promega, Madison, Wis.), and 1 mg total RNA. The samples were incubated at 20° C. for 10 min and at 42° C. for 1 h, and reverse transcriptase was inactivated by heating at 95° C. for 5 min and cooling at 5° C. for 5 min.

(11)PCR Amplification

342. All of the PCR reactions were performed using an ABI Prism 7700 Sequence Detection System (Perkin-Elmer Applied Bio systems). PCR was performed using the SYBR Green PCR Core Reagents kit (Perkin-Elmer Applied Bio systems). The amplification reactions were performed in 25 ml final volume containing 10× SYBR buffer, 25 mM of MgCl₂, 12.5 mM of dNTP, 0.625 units of AmpliTaq Gold and 0.25 units UNG. Final AR, PSA and beta-actin forward, reverse concentrations were 2.5 μM. To reduce variability between replicates, PCR premixes, which contained all reagents expect for total RNA, were prepared and aliquoted into 1.5 ml microfuge tubes. The thermal cycling conditions comprised an initial denaturation step at 95° C. for 10 min and 40 cycles at 95° C. for 15 sec and 60° C. for 1 min. Specific PCR amplification products were detected by the fluorescent double-stranded DNA-binding dye SYBR Green core reagent kit (Perkin-Elmer Applied Bio systems). Experiments were performed with duplicates for each data point.

1. A method of identifying a compound that modulates androgen receptor activity comprising,
a) administering a compound to a system, wherein the system has androgen receptor activity; and wherein the system comprises a protein which interacts with the androgen receptor
b) assaying the effect of the compound on the amount of androgen receptor activity in the system; and
c) selecting a compound which causes a change in the amount of androgen receptor activity in the system.

2. A method of modulating androgen receptor activity comprising administering a compound, wherein the compound causes modulation of androgen receptor activity, wherein the compound is defined as a compound capable of being identified by administering the compound to a system, wherein the system has androgen receptor activity; and wherein the system comprises a protein which interacts with the androgen receptor, assaying the effect of the compound on the amount of androgen receptor activity in the system; and selecting the compound if it causes a change in the amount of androgen receptor activity in the system.

3. A method of modulating androgen receptor activity comprising administering a compound that causes an inhibition of an interaction between androgen receptor and a protein selected from the group consisting of Smad3, Smad4, Akt, TGF-B, and PTEN or fragment thereof.

4. A method of making a composition capable of modulating androgen receptor activity comprising mixing an androgen receptor modulating compound with a pharmaceutically acceptable carrier, wherein the compound is identified by administering the compound to a system, wherein the system has androgen receptor activity; and wherein the system comprises a protein which interacts with the androgen receptor, assaying the effect of the compound on the amount of androgen receptor activity in the system; and selecting the compound if it causes a change in the amount of androgen receptor activity in the system.

5. A method of making a compound that modulates androgen receptor activity comprising,
a) administering a compound to a system, wherein the system has androgen receptor activity; and wherein the system comprises a protein which interacts with the androgen receptor
b) assaying the effect of the compound on the amount of androgen receptor activity in the system; and
c) selecting a compound which causes a change in the amount of androgen receptor activity in the system., and
d) synthesizing the compound.

6. A method of modulating androgen receptor activity comprising administering a compound, wherein the compound is identified as changing the amount of androgen receptor activity in a system.

7. The method of claim 1, wherein the change in the amount of androgen receptor activity is a decrease in the activity.

8. The method of claim 1, wherein the change in the amount of androgen receptor activity is an increase in the activity.

9. The method of claim 1, wherein the activity is the cellular proliferation activity of androgen receptor.

10. The method of claim 1, wherein the activity is the apoptotic activity of androgen receptor.

11. The method of claim 1, wherein the activity is the transcription activation activity of androgen receptor.

12. The method of claim 1, wherein the activity is the PSA altering activity of androgen receptor.

13. The method of claim 1, wherein the modulation is an inhibition.

14. The method of claim 1, wherein the modulation is an enhancement.

15. The method of claim 1, wherein the system is a cell.

16. The method of claim 15, wherein the cell comprises a nucleic acid encoding a first protein, selected from the group consisting of AR, Smad3, Smad4, Akt, PTEN, and TGF-B such that expression of first protein mRNA can occur.

17. The method of claim 15, wherein the system further comprises a nucleic acid encoding a second protein selected from the group consisting of AR, Smad3, Smad4, Akt, PTEN, and TGF-B, such that expression of second protein mRNA can occur.

18. The method of claim 15, wherein the system further comprises a nucleic acid encoding Smad3.

19. The method of claim 18, wherein the system further comprises a nucleic acid encoding Smad4.

20. The method of claim 16, wherein the expression of the first protein mRNA is regulatable, constitutive, or inducible.

21. The method of claim 17, wherein the expression of the second protein mRNA is regulatable, constitutive, or inducible.

22. The method of claim 20, wherein the expression of the first protein mRNA is regulatable, constitutive, or inducible.

23. A method of identifying a modulator of an interaction between androgen receptor and Smad3 comprising
a) administering a compound to a system, wherein the system comprises Smad3 and androgen receptor,
b) assaying the effect of the compound on a Smad3-androgen receptor interaction, and
c) selecting a compound which modulates the Smad3-androgen receptor interaction.

24. A method of identifying a modulator of an interaction between androgen receptor and Smad4 comprising
a) administering a compound to a system, wherein the system comprises Smad4 and androgen receptor,
b) assaying the effect of the compound on a Smad4-androgen receptor interaction, and
c) selecting a compound which modulates the Smad4-androgen receptor interaction.

25. A method of identifying a modulator of an interaction between androgen receptor and Akt comprising
a) administering a compound to a system, wherein the system comprises Akt and androgen receptor,
b) assaying the effect of the compound on a Akt-androgen receptor interaction, and
c) selecting a compound which modulates the Akt-androgen receptor interaction.

26. A method of identifying a modulator of an interaction between androgen receptor and PTEN comprising
a) administering a compound to a system, wherein the system comprises Smad3 and androgen receptor,
b) assaying the effect of the compound on a PTEN-androgen receptor interaction, and
c) selecting a compound which inhibits the PTEN-androgen receptor interaction.

27. A cell comprising,
a) a regulatable nucleic acid comprising sequence encoding an AR gene and
b) a nucleic acid comprising sequence encoding a Smad3 gene.

28. A cell comprising,
a) a regulatable nucleic acid comprising sequence encoding an AR gene and
b) a nucleic acid comprising sequence encoding a Smad4 gene.

29. A cell comprising,
a) a regulatable nucleic acid comprising sequence encoding an AR gene and
b) a nucleic acid comprising sequence encoding a Akt gene.

30. A cell comprising,
a) a regulatable nucleic acid comprising sequence encoding an AR gene and
b) a nucleic acid comprising sequence encoding a PTEN gene.

31. A cell comprising,
a) a regulatable nucleic acid comprising sequence encoding an AR gene and
b) a nucleic acid comprising sequence encoding a TGF-B gene.

32. A method for modulating androgen receptor-mediated transactivation activity in a cell, comprising the step of:
treating the cell with an agent that modulates the activity of a pathway from or to androgen receptor-androgen receptor coactivator interaction.
